SEQUENCE LISTING (206 sequences)

SEQ ID NO: 1 - DNA, 40 bp, Artificial Sequence
  Description of Artificial Sequence Synthetic oligonucleotide
gtgtattctacagtgcacgtgtctccagtgtggctcggag

SEQ ID NO: 2 - RNA, 22 nt, Artificial Sequence
  Description of Artificial Sequence Synthetic oligonucleotide
ucuacagugcacgugucuccag

SEQ ID NO: 3 - RNA, 68 nt, Artificial Sequence
  Description of Artificial Sequence Synthetic oligonucleotide
guguauucuacagugcacgugucuccaguguggcucggaggcuggagacgcggcccuguuggaguaac

SEQ ID NO: 4 - RNA, 110 nt, Artificial Sequence
  Description of Artificial Sequence Synthetic polynucleotide
cuggauacagaguggaccggcuggccccaucuggaagacuagugauuuuguuguugucuuacugcgcucaacaacaaaucccagucuaccuaauggugccagccaucgca

SEQ ID NO: 5 - DNA, 81 bp, Artificial Sequence
  Description of Artificial Sequence Synthetic oligonucleotide
accttgtcgggtagcttatcagactgatgttgactgttgaatctcatggcaacaccagtcgatgggctgtctgacattttg

SEQ ID NO: 6 - DNA, 40 bp, Artificial Sequence
  Description of Artificial Sequence Synthetic oligonucleotide
ttcaacagtcaacatcagtctgataagctacccgacaagg

SEQ ID NO: 7 - RNA, 22 nt, Artificial Sequence
  Description of Artificial Sequence Synthetic oligonucleotide
uagcuuaucagacugauguuga

SEQ ID NO: 8 - RNA, 72 nt, Artificial Sequence
  Description of Artificial Sequence Synthetic oligonucleotide
ugucggguagcuuaucagacugauguugacuguugaaucucauggcaacaccagucgaugggcugucugaca

SEQ ID NO: 9 - DNA, 40 bp, Artificial Sequence
  Description of Artificial Sequence Synthetic oligonucleotide
gaatcagtcaccatcagttcctaatgcattgccttcagca

SEQ ID NO: 10 - RNA, 22 nt, Artificial Sequence
  Description of Artificial Sequence Synthetic oligonucleotide
gcugacuccuaguccagggcuc

SEQ ID NO: 11 - RNA, 110 nt, Artificial Sequence
  Description of Artificial Sequence Synthetic polynucleotide
aguauaauuauuacauaguuuuugaugucgcagauacugcaucaggaacugauuggauaagaaucagucaccaucaguuccuaaugcauugccuucagcaucuaaacaag

SEQ ID NO: 12 - DNA, 40 bp, Artificial Sequence
  Description of Artificial Sequence Synthetic oligonucleotide
tcgctgcggggctttcctttgtgcttgatctaaccatgtg

SEQ ID NO: 13 - DNA, 78 bp, Artificial Sequence
  Description of Artificial Sequence Synthetic oligonucleotide
tggccgattttggcactagcacatttttgcttgtgtctctccgctctgagcaatcatgtgcagtgccaatatgggaaa

SEQ ID NO: 14 - DNA, 40 bp, Artificial Sequence
  Description of Artificial Sequence Synthetic oligonucleotide
ctccgctctgagcaatcatgtgcagtgccaatatgggaaa

SEQ ID NO: 15 - RNA, 78 nt, Artificial Sequence
  Description of Artificial Sequence Synthetic oligonucleotide
uggccgauuuuggcacuagcacauuuuugcuugugucucuccgcucugagcaaucaugugcagugccaauaugggaaa

SEQ ID NO: 16 - DNA, 40 bp, Artificial Sequence
  Description of Artificial Sequence Synthetic oligonucleotide
cagggtcacaggtgaggttcttgggagcctggcgtctggc

SEQ ID NO: 17 - RNA, 86 nt, Artificial Sequence
  Description of Artificial Sequence Synthetic oligonucleotide
ugccagucucuaggucccugagacccuuuaaccugugaggacauccagggucacaggugagguucuugggagccuggcgucuggcc

SEQ ID NO: 18 - DNA, 40 bp, Artificial Sequence
  Description of Artificial Sequence Synthetic oligonucleotide
ttgtgaagctcctaacactgtctggtaaagatggctcccg

SEQ ID NO: 19 - RNA, 95 nt, Artificial Sequence
  Description of Artificial Sequence Synthetic oligonucleotide
cggccggcccuggguccaucuuccaguacaguguuggauggucuaauugugaagcuccuaacacugucugguaaagauggcucccggguggguuc

SEQ ID NO: 20 - DNA, 40 bp, Artificial Sequence
  Description of Artificial Sequence Synthetic oligonucleotide
tggtggaacgatggaaacggaacatggttctgtcaagcac

SEQ ID NO: 21 - RNA, 21 nt, Artificial Sequence
  Description of Artificial Sequence Synthetic oligonucleotide
uugugcuugaucuaaccaugu

SEQ ID NO: 22 - RNA, 110 nt, Artificial Sequence
  Description of Artificial Sequence Synthetic polynucleotide
gaccagucgcugcggggcuuuccuuugugcuugaucuaaccaugugguggaacgauggaaacggaacaugguucugucaagcaccgcggaaagcaccgugcucuccugca

SEQ ID NO: 23 - DNA, 40 bp, Artificial Sequence
  Description of Artificial Sequence Synthetic oligonucleotide
ctaggaagaggtagtagtttgcatagttttagggcaaaga

SEQ ID NO: 24 - RNA, 22 nt, Artificial Sequence
  Description of Artificial Sequence Synthetic oligonucleotide
agagguaguagguugcauaguu

SEQ ID NO: 25 - RNA, 87 nt, Artificial Sequence
  Description of Artificial Sequence Synthetic oligonucleotide
ccuaggaagagguaguagguugcauaguuuuagggcagggauuuugcccacaaggagguaacuauacgaccugcugccuuucuuagg

SEQ ID NO: 26 - DNA, 40 bp, Artificial Sequence
  Description of Artificial Sequence Synthetic oligonucleotide
gacgggacattattacttttggtacgcgctgtgacacttc

SEQ ID NO: 27 - RNA, 22 nt, Artificial Sequence
  Description of Artificial Sequence Synthetic oligonucleotide
ucguaccgugaguaauaaugcg

SEQ ID NO: 28 - RNA, 85 nt, Artificial Sequence
  Description of Artificial Sequence Synthetic oligonucleotide
cgcuggcgacgggacauuauuacuuuugguacgcgcugugacacuucaaacucguaccgugaguaauaaugcgccguccacggca

SEQ ID NO: 29 - DNA, 40 bp, Artificial Sequence
  Description of Artificial Sequence Synthetic oligonucleotide
ctccccatggccctgtctcccaacccttgtaccagtgctg

SEQ ID NO: 30 - RNA, 22 nt, Artificial Sequence
  Description of Artificial Sequence Synthetic oligonucleotide
ucucccaacccuuguaccagug

SEQ ID NO: 31 - RNA, 84 nt, Artificial Sequence
  Description of Artificial Sequence Synthetic oligonucleotide
cuccccauggcccugucucccaacccuuguaccagugcugggcucagacccugguacaggccugggggacagggaccuggggac

SEQ ID NO: 32 - DNA, 40 bp, Artificial Sequence
  Description of Artificial Sequence Synthetic oligonucleotide
gtcgggtagcttatcagactgatgttgactgttgaatctc

SEQ ID NO: 33 - RNA, 72 nt, Artificial Sequence; identical to SEQ ID NO: 8
  Description of Artificial Sequence Synthetic oligonucleotide

SEQ ID NO: 34 - DNA, 40 bp, Artificial Sequence
  Description of Artificial Sequence Synthetic oligonucleotide
gtcagaataatgtcaaagtgcttacagtgcaggtagtgat

SEQ ID NO: 35 - RNA, 23 nt, Artificial Sequence
  Description of Artificial Sequence Synthetic oligonucleotide
caaagugcuuacagugcagguag

SEQ ID NO: 36 - RNA, 84 nt, Artificial Sequence
  Description of Artificial Sequence Synthetic oligonucleotide
gucagaauaaugucaaagugcuuacagugcagguagugauaugugcaucuacugcagugaaggcacuuguagcauuauggugac

SEQ ID NO: 37 - DNA, 40 bp, Artificial Sequence
  Description of Artificial Sequence Synthetic oligonucleotide
taagctatggaatgtaaagaagtatgtatctcaggccggg

SEQ ID NO: 38 - RNA, 22 nt, Artificial Sequence
  Description of Artificial Sequence Synthetic oligonucleotide
uggaauguaaagaaguauguau

SEQ ID NO: 39 - RNA, 71 nt, Artificial Sequence
  Description of Artificial Sequence Synthetic oligonucleotide
ugggaaacauacuucuuuauaugcccauauggaccugcuaagcuauggaauguaaagaaguauguaucuca

SEQ ID NO: 40 - DNA, 40 bp, Artificial Sequence
  Description of Artificial Sequence Synthetic oligonucleotide
gatctggcctaaagaggtatagggcatgggaagatggagc

SEQ ID NO: 41 - RNA, 20 nt, Artificial Sequence
  Description of Artificial Sequence Synthetic oligonucleotide
agagguauagggcaugggaa

SEQ ID NO: 42 - RNA, 110 nt, Artificial Sequence
  Description of Artificial Sequence Synthetic polynucleotide
cgccucagagccgcccgccguuccuuuuuccuaugcauauacuucuuugaggaucuggccuaaagagguauagggcaugggaaaacggggcggucggguccuccccagcg

SEQ ID NO: 43 - DNA, 40 bp, Artificial Sequence
  Description of Artificial Sequence Synthetic oligonucleotide
taaagtgcttatagtgcaggtagtgtttagttatctactg

SEQ ID NO: 44 - DNA, 40 bp, Artificial Sequence
  Description of Artificial Sequence Synthetic oligonucleotide
gctgaggtagtagtttgtgctgttggtcgggttgtgacat

SEQ ID NO: 45 - RNA, 22 nt, Artificial Sequence
  Description of Artificial Sequence Synthetic oligonucleotide
ugagguaguaguuugugcuguu

SEQ ID NO: 46 - RNA, 84 nt, Artificial Sequence
  Description of Artificial Sequence Synthetic oligonucleotide
cuggcugagguaguaguuugugcuguuggucggguugugacauugcccgcuguggagauaacugcgcaagcuacugccuugcua

SEQ ID NO: 47 - DNA, 40 bp, Artificial Sequence
  Description of Artificial Sequence Synthetic oligonucleotide
ctgattccaggctgaggtagtagtttgtacagtttgaggg

SEQ ID NO: 48 - DNA, 40 bp, Artificial Sequence
  Description of Artificial Sequence Synthetic oligonucleotide
taaggtgcatctagtgcagatagtgaagtagattagcatc

SEQ ID NO: 49 - DNA, 40 bp, Artificial Sequence
  Description of Artificial Sequence Synthetic oligonucleotide
ttgctatggaatgtaaggaagtgtgtggtttcggcaagtg

SEQ ID NO: 50 - RNA, 22 nt, Artificial Sequence
  Description of Artificial Sequence Synthetic oligonucleotide
uggaauguaaggaagugugugg

SEQ ID NO: 51 - RNA, 86 nt, Artificial Sequence
  Description of Artificial Sequence Synthetic oligonucleotide
ugcuucccgaggccacaugcuucuuuauauccccauauggauuacuuugcuauggaauguaaggaagugugugguuucggcaagug

SEQ ID NO: 52 - DNA, 40 bp, Artificial Sequence
  Description of Artificial Sequence Synthetic oligonucleotide
tctttggttatctagctgtatgagtggtgtggagtcttca

SEQ ID NO: 53 - RNA, 23 nt, Artificial Sequence
  Description of Artificial Sequence Synthetic oligonucleotide
ucuuugguuaucuagcuguauga

SEQ ID NO: 54 - RNA, 89 nt, Artificial Sequence
  Description of Artificial Sequence Synthetic oligonucleotide
cgggguugguuguuaucuuugguuaucuagcuguaugagugguguggagucuucauaaagcuagauaaccgaaaguaaaaauaacccca

SEQ ID NO: 55 - DNA, 40 bp, Artificial Sequence
  Description of Artificial Sequence Synthetic oligonucleotide
atcgaccgttgagtggaccctgaggcctggaattgccatc

SEQ ID NO: 56 - RNA, 22 nt, Artificial Sequence
  Description of Artificial Sequence Synthetic oligonucleotide
aacauucaaccugucggugagu

SEQ ID NO: 57 - RNA, 110 nt, Artificial Sequence
  Description of Artificial Sequence Synthetic polynucleotide
cggaaaauuugccaaggguuugggggaacauucaaccugucggugaguuugggcagcucaggcaaaccaucgaccguugaguggacccugaggccuggaauugccauccu

SEQ ID NO: 58 - DNA, 40 bp, Artificial Sequence
  Description of Artificial Sequence Synthetic oligonucleotide
tgaggtagtaggttgtgtggtttcagggcagtgatgttgc

SEQ ID NO: 59 - RNA, 22 nt, Artificial Sequence
  Description of Artificial Sequence Synthetic oligonucleotide
ugagguaguagguugugugguu

SEQ ID NO: 60 - RNA, 83 nt, Artificial Sequence
  Description of Artificial Sequence Synthetic oligonucleotide
cggggugagguaguagguugugugguuucagggcagugauguugccccucggaagauaacuauacaaccuacugccuucccug

SEQ ID NO: 61 - DNA, 40 bp, Artificial Sequence
  Description of Artificial Sequence Synthetic oligonucleotide
agattagagtggctgtggtctagtgctgtgtggaagacta

SEQ ID NO: 62 - RNA, 23 nt, Artificial Sequence
  Description of Artificial Sequence Synthetic oligonucleotide
uggaagacuagugauuuuguugu

SEQ ID NO: 63 - RNA, 110 nt, Artificial Sequence
  Description of Artificial Sequence Synthetic polynucleotide
agauuagaguggcuguggucuagugcuguguggaagacuagugauuuuguuguucugauguacuacgacaacaagucacagccggccucauagcgcagacucccuucgac

SEQ ID NO: 64 - DNA, 40 bp, Artificial Sequence
  Description of Artificial Sequence Synthetic oligonucleotide
gtagcattcaggtcaagcaacattgtacagggctatgaaa

SEQ ID NO: 65 - RNA, 23 nt, Artificial Sequence
  Description of Artificial Sequence Synthetic oligonucleotide
agcagcauuguacagggcuauga

SEQ ID NO: 66 - RNA, 78 nt, Artificial Sequence
  Description of Artificial Sequence Synthetic oligonucleotide
uugugcuuucagcuucuuuacagugcugccuuguagcauucaggucaagcagcauuguacagggcuaugaaagaacca

SEQ ID NO: 67 - DNA, 40 bp, Artificial Sequence
  Description of Artificial Sequence Synthetic oligonucleotide
caatgtcagcagtgccttagcagcacgtaaatattggcgt

SEQ ID NO: 68 - RNA, 22 nt, Artificial Sequence
  Description of Artificial Sequence Synthetic oligonucleotide
uagcagcacguaaauauuggcg

SEQ ID NO: 69 - RNA, 89 nt, Artificial Sequence
  Description of Artificial Sequence Synthetic oligonucleotide
gucagcagugccuuagcagcacguaaauauuggcguuaagauucuaaaauuaucuccaguauuaacugugcugcugaaguaagguugac

SEQ ID NO: 70 - DNA, 40 bp, Artificial Sequence
  Description of Artificial Sequence Synthetic oligonucleotide
ccttggccatgtaaaagtgcttacagtgcaggtagctttt

SEQ ID NO: 71 - RNA, 23 nt, Artificial Sequence
  Description of Artificial Sequence Synthetic oligonucleotide
aaaagugcuuacagugcagguag

SEQ ID NO: 72 - RNA, 81 nt, Artificial Sequence
  Description of Artificial Sequence Synthetic oligonucleotide
ccuuggccauguaaaagugcuuacagugcagguagcuuuuugagaucuacugcaauguaagcacuucuuacauuaccaugg

SEQ ID NO: 73 - DNA, 40 bp, Artificial Sequence
  Description of Artificial Sequence Synthetic oligonucleotide
ctgctgagtgaattaggtagtttcatgttgttgggcctgg

SEQ ID NO: 74 - RNA, 22 nt, Artificial Sequence
  Description of Artificial Sequence Synthetic oligonucleotide
uagguaguuucauguuguuggg

SEQ ID NO: 75 - RNA, 70 nt, Artificial Sequence
  Description of Artificial Sequence Synthetic oligonucleotide
gugaauuagguaguuucauguuguugggccuggguuucugaacacaacaacauuaaaccacccgauucac

SEQ ID NO: 76 - DNA, 40 bp, Artificial Sequence
  Description of Artificial Sequence Synthetic oligonucleotide
gggatgaggtagtagattgtatagttgtggggtagtgatt

SEQ ID NO: 77 - RNA, 22 nt, Artificial Sequence
  Description of Artificial Sequence Synthetic oligonucleotide
ugagguaguagauuguauaguu

SEQ ID NO: 78 - RNA, 87 nt, Artificial Sequence
  Description of Artificial Sequence Synthetic oligonucleotide
ucagagugagguaguagauuguauaguugugggguagugauuuuacccuguucaggagauaacuauacaaucuauugccuucccuga

SEQ ID NO: 79 - DNA, 81 bp, Artificial Sequence
  Description of Artificial Sequence Synthetic oligonucleotide
ctctctgctttcagcttctttacagtgttgccttgtggcatggagttcaagcagcattgtacagggctatcaaagcacaga

SEQ ID NO: 80 - DNA, 40 bp, Artificial Sequence
  Description of Artificial Sequence Synthetic oligonucleotide
ggcatggagttcaagcagcattgtacagggctatcaaagc

SEQ ID NO: 81 - RNA, 23 nt, Artificial Sequence
  Description of Artificial Sequence Synthetic oligonucleotide
agcagcauuguacagggcuauca

SEQ ID NO: 82 - RNA, 81 nt, Artificial Sequence
  Description of Artificial Sequence Synthetic oligonucleotide
cucucugcuuucagcuucuuuacaguguugccuuguggcauggaguucaagcagcauuguacagggcuaucaaagcacaga

SEQ ID NO: 83 - DNA, 40 bp, Artificial Sequence
  Description of Artificial Sequence Synthetic oligonucleotide
tgaggtagtaggttgtatagttttagggtcacacccacca

SEQ ID NO: 84 - RNA, 22 nt, Artificial Sequence
  Description of Artificial Sequence Synthetic oligonucleotide
ugagguaguagguuguauaguu

SEQ ID NO: 85 - RNA, 80 nt, Artificial Sequence
  Description of Artificial Sequence Synthetic oligonucleotide
ugggaugagguaguagguuguauaguuuuagggucacacccaccacugggagauaacuauacaaucuacugucuuuccua

SEQ ID NO: 86 - DNA, 40 bp, Artificial Sequence
  Description of Artificial Sequence Synthetic oligonucleotide
ccctggctgggatatcatcatatactgtaagtttgcgatg

SEQ ID NO: 87 - RNA, 20 nt, Artificial Sequence
  Description of Artificial Sequence Synthetic oligonucleotide
uacaguauagaugauguacu

SEQ ID NO: 88 - RNA, 86 nt, Artificial Sequence
  Description of Artificial Sequence Synthetic oligonucleotide
uggggcccuggcugggauaucaucauauacuguaaguuugcgaugagacacuacaguauagaugauguacuaguccgggcaccccc

SEQ ID NO: 89 - DNA, 40 bp, Artificial Sequence
  Description of Artificial Sequence Synthetic oligonucleotide
cctaggaagaggtagtaggttgcatagttttagggcaggg

SEQ ID NO: 90 - RNA, 22 nt, Artificial Sequence
  Description of Artificial Sequence Synthetic oligonucleotide
agagguaguagguugcauaguu

SEQ ID NO: 91 - RNA, 87 nt, Artificial Sequence; identical to SEQ ID NO: 25
  Description of Artificial Sequence Synthetic oligonucleotide

SEQ ID NO: 92 - DNA, 40 bp, Artificial Sequence
  Description of Artificial Sequence Synthetic oligonucleotide
tatggatcaagcagcattgtacagggctatgaaggcattg

SEQ ID NO: 93 - RNA, 23 nt, Artificial Sequence
  Description of Artificial Sequence Synthetic oligonucleotide
agcagcauuguacagggcuauga

SEQ ID NO: 94 - RNA, 78 nt, Artificial Sequence
  Description of Artificial Sequence Synthetic oligonucleotide
uacugcccucggcuucuuuacagugcugccuuguugcauauggaucaagcagcauuguacagggcuaugaaggcauug

SEQ ID NO: 95 - DNA, 40 bp, Artificial Sequence
  Description of Artificial Sequence Synthetic oligonucleotide
aatgctatggaatgtaaagaagtatgtatttttggtaggc

SEQ ID NO: 96 - RNA, 22 nt, Artificial Sequence
  Description of Artificial Sequence Synthetic oligonucleotide
uggaauguaaagaaguauguau

SEQ ID NO: 97 - RNA, 71 nt, Artificial Sequence; identical to SEQ ID NO: 39
  Description of Artificial Sequence Synthetic oligonucleotide

SEQ ID NO: 98 - DNA, 40 bp, Artificial Sequence
  Description of Artificial Sequence Synthetic oligonucleotide
accagacttttcctagtccctgagaccctaacttgtgagg

SEQ ID NO: 99 - RNA, 22 nt, Artificial Sequence
  Description of Artificial Sequence Synthetic oligonucleotide
ucccugagacccuaacuuguga

SEQ ID NO: 100 - RNA, 89 nt, Artificial Sequence
  Description of Artificial Sequence Synthetic oligonucleotide
accagacuuuuccuagucccugagacccuaacuugugagguauuuuaguaacaucacaagucaggcucuugggaccuaggcggagggga

SEQ ID NO: 101 - DNA, 40 bp, Artificial Sequence
  Description of Artificial Sequence Synthetic oligonucleotide
tgtctgcacctgtcactagcagtgcaatgttaaaagggca

SEQ ID NO: 102 - RNA, 22 nt, Artificial Sequence
  Description of Artificial Sequence Synthetic oligonucleotide
cagugcaauguuaaaagggcau

SEQ ID NO: 103 - RNA, 89 nt, Artificial Sequence
  Description of Artificial Sequence Synthetic oligonucleotide
ugcugcuggccagagcucuuuucacauugugcuacugucugcaccugucacuagcagugcaauguuaaaagggcauuggccguguagug

SEQ ID NO: 104 - DNA, 40 bp, Artificial Sequence
  Description of Artificial Sequence Synthetic oligonucleotide
atggattggctgggaggtggatgtttacttcagctgactt

SEQ ID NO: 105 - RNA, 22 nt, Artificial Sequence
  Description of Artificial Sequence Synthetic oligonucleotide
uguaaacauccuacacucagcu

SEQ ID NO: 106 - RNA, 88 nt, Artificial Sequence
  Description of Artificial Sequence Synthetic oligonucleotide
accaaguuucaguucauguaaacauccuacacucagcuguaauacauggauuggcugggagguggauguuuacuucagcugacuugga

SEQ ID NO: 107 - DNA, 40 bp, Artificial Sequence
  Description of Artificial Sequence Synthetic oligonucleotide
actgcatgctcccaggttgaggtagtaggttgtatagttt

SEQ ID NO: 108 - RNA, 22 nt, Artificial Sequence
  Description of Artificial Sequence Synthetic oligonucleotide
ugagguaguagguuguauaguu

SEQ ID NO: 109 - RNA, 72 nt, Artificial Sequence
  Description of Artificial Sequence Synthetic oligonucleotide
agguugagguaguagguuguauaguuuagaauuacaucaagggagauaacuguacagccuccuagcuuuccu

SEQ ID NO: 110 - DNA, 40 bp, Artificial Sequence
  Description of Artificial Sequence Synthetic oligonucleotide
tccagggcaaccgtggctttcgattgttactgtgggaact

SEQ ID NO: 111 - RNA, 22 nt, Artificial Sequence
  Description of Artificial Sequence Synthetic oligonucleotide
uaacagucuacagccauggucg

SEQ ID NO: 112 - RNA, 101 nt, Artificial Sequence
  Description of Artificial Sequence Synthetic polynucleotide
ccgcccccgcgucuccagggcaaccguggcuuucgauuguuacugugggaacuggagguaacagucuacagccauggucgccccgcagcacgcccacgcgc

SEQ ID NO: 113 - DNA, 40 bp, Artificial Sequence
  Description of Artificial Sequence Synthetic oligonucleotide
ccttggagtaaagtagcagcacataatggtttgtggattt

SEQ ID NO: 114 - RNA, 22 nt, Artificial Sequence
  Description of Artificial Sequence Synthetic oligonucleotide
uagcagcacauaaugguuugug

SEQ ID NO: 115 - RNA, 83 nt, Artificial Sequence
  Description of Artificial Sequence Synthetic oligonucleotide
ccuuggaguaaaguagcagcacauaaugguuuguggauuuugaaaaggugcaggccauauugugcugccucaaaaauacaagg

SEQ ID NO: 116 - DNA, 81 bp, Artificial Sequence
  Description of Artificial Sequence Synthetic oligonucleotide
gttccactctagcagcacgtaaatattggcgtagtgaaatatatattaaacaccaatattactgtgctgctttagtgtgac

SEQ ID NO: 117 - DNA, 40 bp, Artificial Sequence
  Description of Artificial Sequence Synthetic oligonucleotide
gttccactctagcagcacgtaaatattggcgtagtgaaat

SEQ ID NO: 118 - RNA, 22 nt, Artificial Sequence
  Description of Artificial Sequence Synthetic oligonucleotide
aucgugcaucccuuuagagugu

SEQ ID NO: 119 - RNA, 87 nt, Artificial Sequence
  Description of Artificial Sequence Synthetic oligonucleotide
ucucaggcagugacccucuagauggaagcacugucuguuguauaaaagaaaagaucgugcaucccuuuagaguguuacuguuugaga

SEQ ID NO: 120 - DNA, 40 bp, Artificial Sequence
  Description of Artificial Sequence Synthetic oligonucleotide
gcatctactgcagtgaaggcacttgtagcattatggtgac

SEQ ID NO: 121 - RNA, 23 nt, Artificial Sequence
  Description of Artificial Sequence Synthetic oligonucleotide
caaagugcuuacagugcagguag

SEQ ID NO: 122 - RNA, 84 nt, Artificial Sequence; identical to SEQ ID NO: 36
  Description of Artificial Sequence Synthetic oligonucleotide

SEQ ID NO: 123 - RNA, 22 nt, Artificial Sequence
  Description of Artificial Sequence Synthetic oligonucleotide
aguauucuguaccagggaaggu

SEQ ID NO: 124 - RNA, 97 nt, Artificial Sequence
  Description of Artificial Sequence Synthetic oligonucleotide
aacuuaacaucaugcuaccucuuuguaucauauuuuguuauucuggucacagaaugaccuaguauucuguaccagggaagguaguucuuaacuauau

SEQ ID NO: 125 - RNA, 21 nt, Artificial Sequence
  Description of Artificial Sequence Synthetic oligonucleotide
cugaccuaugaauugacagcc

SEQ ID NO: 126 - RNA, 110 nt, Artificial Sequence
  Description of Artificial Sequence Synthetic polynucleotide
gccgagaccgagugcacagggcucugaccuaugaauugacagccagugcucucgucuccccucuggcugccaauuccauaggucacagguauguucgccucaaugccagc

SEQ ID NO: 127 - RNA, 21 nt, Artificial Sequence
  Description of Artificial Sequence Synthetic oligonucleotide
cugaccuaugaauugacagcc

SEQ ID NO: 128 - RNA, 91 nt, Artificial Sequence
  Description of Artificial Sequence Synthetic oligonucleotide
cgacuugcuuucucuccuccaugccuugaguguaggaccguuggcaucuuaauuacccucccacacccaaggcuugcaaaaaagcgagccu

SEQ ID NO: 129 - RNA, 22 nt, Artificial Sequence
  Description of Artificial Sequence Synthetic oligonucleotide
caugccuugaguguaggaccgu

SEQ ID NO: 130 - RNA, 75 nt, Artificial Sequence
  Description of Artificial Sequence Synthetic oligonucleotide
ggcugugccggguagagagggcagugggagguaagagcucuucacccuucaccaccuucuccacccagcauggcc

SEQ ID NO: 131 - RNA, 22 nt, Artificial Sequence
  Description of Artificial Sequence Synthetic oligonucleotide
uucaccaccuucuccacccagc

SEQ ID NO: 132 - RNA, 99 nt, Artificial Sequence
  Description of Artificial Sequence Synthetic oligonucleotide
gaaacugggcucaaggugaggggugcuaucugugauugagggacaugguuaauggaauugucucacacagaaaucgcacccgucaccuuggccuacuua

SEQ ID NO: 133 - RNA, 22 nt, Artificial Sequence
  Description of Artificial Sequence Synthetic oligonucleotide
uaaugccccuaaaaauccuuau

SEQ ID NO: 134 - RNA, 22 nt, Artificial Sequence
  Description of Artificial Sequence Synthetic oligonucleotide
aaucauacagggacauccaguu

SEQ ID NO: 135 - RNA, 80 nt, Artificial Sequence
  Description of Artificial Sequence Synthetic oligonucleotide
gguacuugaagagugguuaucccugcuguguucgcuuaauuuaugacgaaucauacagggacauccaguuuuucaguauc

SEQ ID NO: 136 - RNA, 22 nt, Artificial Sequence
  Description of Artificial Sequence Synthetic oligonucleotide
ucagugcacuacagaacuuugu

SEQ ID NO: 137 - RNA, 68 nt, Artificial Sequence
  Description of Artificial Sequence Synthetic oligonucleotide
gaggcaaaguucugagacacuccgacucugaguaugauagaagucagugcacuacagaacuuugucuc

SEQ ID NO: 138 - RNA, 22 nt, Artificial Sequence
  Description of Artificial Sequence Synthetic oligonucleotide
ucagugcacuacagaacuuugu

SEQ ID NO: 139 - RNA, 23 nt, Artificial Sequence
  Description of Artificial Sequence Synthetic oligonucleotide
guccaguuuucccaggaaucccu

SEQ ID NO: 140 - RNA, 88 nt, Artificial Sequence
  Description of Artificial Sequence Synthetic oligonucleotide
caccuuguccucacgguccaguuuucccaggaaucccuuagaugcuaagauggggauuccuggaaauacuguucuugaggucaugguu

SEQ ID NO: 141 - RNA, 23 nt, Artificial Sequence
  Description of Artificial Sequence Synthetic oligonucleotide
guccaguuuucccaggaaucccu

SEQ ID NO: 142 - RNA, 87 nt, Artificial Sequence
  Description of Artificial Sequence Synthetic oligonucleotide
ugucccccccggcccagguucugugauacacuccgacucgggcucuggagcagucagugcaugacagaacuugggcccggaaggacc

SEQ ID NO: 143 - RNA, 22 nt, Artificial Sequence
  Description of Artificial Sequence Synthetic oligonucleotide
aaaagcuggguugagagggcga

SEQ ID NO: 144 - RNA, 82 nt, Artificial Sequence
  Description of Artificial Sequence Synthetic oligonucleotide
gcuucgcuccccuccgccuucucuucccgguucuucccggagucgggaaaagcuggguugagagggcgaaaaaggaugaggu

SEQ ID NO: 145 - RNA, 22 nt, Artificial Sequence
  Description of Artificial Sequence Synthetic oligonucleotide
aaaagcuggguugagagggcga

SEQ ID NO: 146 - RNA, 82 nt, Artificial Sequence; identical to SEQ ID NO: 144
  Description of Artificial Sequence Synthetic oligonucleotide

SEQ ID NO: 147 - RNA, 22 nt, Artificial Sequence
  Description of Artificial Sequence Synthetic oligonucleotide
aaaagcuggguugagagggcaa

SEQ ID NO: 148 - RNA, 79 nt, Artificial Sequence
  Description of Artificial Sequence Synthetic oligonucleotide
aauuaaucccucucuuucuaguucuuccuagagugaggaaaagcuggguugagagggcaaacaaauuaacuaauuaauu

SEQ ID NO: 149 - RNA, 22 nt, Artificial Sequence
  Description of Artificial Sequence Synthetic oligonucleotide
aaaagcuggguugagagggcaa

SEQ ID NO: 150 - RNA, 138 nt, Artificial Sequence
  Description of Artificial Sequence Synthetic polynucleotide
uguuauuuuuugucuucuaccuaagaauucugucucuuaggcuuucucuucccagauuucccaaaguugggaaaagcuggguugagagggcaaaaggaaaaaaaaagaauucugucucugacauaauuagauagggaa

SEQ ID NO: 151 - RNA, 20 nt, Artificial Sequence
  Description of Artificial Sequence Synthetic oligonucleotide
aaaagcuggguugagagggu

SEQ ID NO: 152 - RNA, 88 nt, Artificial Sequence
  Description of Artificial Sequence Synthetic oligonucleotide
uuugcauuaaaaaugaggccuucucuucccaguucuucccagagucaggaaaagcuggguugagaggguagaaaaaaaaugauguagg

SEQ ID NO: 153 - RNA, 20 nt, Artificial Sequence
  Description of Artificial Sequence Synthetic oligonucleotide
aaaagcuggguugagagggu

SEQ ID NO: 154 - RNA, 50 nt, Artificial Sequence
  Description of Artificial Sequence Synthetic oligonucleotide
cuucucuuuccaguucuucccagaauugggaaaagcuggguugagagggu

SEQ ID NO: 155 - RNA, 19 nt, Artificial Sequence
  Description of Artificial Sequence Synthetic oligonucleotide
aaaagcuggguugagagga

SEQ ID NO: 156 - RNA, 48 nt, Artificial Sequence
  Description of Artificial Sequence Synthetic oligonucleotide
uucucgucccaguucuucccaaaguugagaaaagcuggguugagagga

SEQ ID NO: 157 - RNA, 21 nt, Artificial Sequence
  Description of Artificial Sequence Synthetic oligonucleotide
gaaagcgcuucucuuuagagg

SEQ ID NO: 158 - RNA, 87 nt, Artificial Sequence
  Description of Artificial Sequence Synthetic oligonucleotide
ucucaugcugugacccucuagagggaagcacuuucucuugucuaaaagaaaagaaagcgcuucucuuuagaggauuacucuuugaga

SEQ ID NO: 159 - RNA, 22 nt, Artificial Sequence
  Description of Artificial Sequence Synthetic oligonucleotide
ucgugcaucccuuuagaguguu

SEQ ID NO: 160 - RNA, 67 nt, Artificial Sequence
  Description of Artificial Sequence Synthetic oligonucleotide
gugacccucuagauggaagcacugucuguugucuaagaaaagaucgugcaucccuuuagaguguuac

SEQ ID NO: 161 - RNA, 21 nt, Artificial Sequence
  Description of Artificial Sequence Synthetic oligonucleotide
uaaagugcugacagugcagau

SEQ ID NO: 162 - RNA, 82 nt, Artificial Sequence
  Description of Artificial Sequence Synthetic oligonucleotide
ccugccggggcuaaagugcugacagugcagauagugguccucuccgugcuaccgcacuguggguacuugcugcuccagcagg

SEQ ID NO: 163 - RNA, 22 nt, Artificial Sequence
  Description of Artificial Sequence Synthetic oligonucleotide
gcugacuccuaguccagggcuc

SEQ ID NO: 164 - RNA, 98 nt, Artificial Sequence
  Description of Artificial Sequence Synthetic oligonucleotide
acccaaacccuaggucugcugacuccuaguccagggcucgugauggcuggugggcccugaacgaggggucuggaggccuggguuugaauaucgacagc

SEQ ID NO: 165 - RNA, 86 nt, Artificial Sequence
  Description of Artificial Sequence Synthetic oligonucleotide
uugguacuuggagagaggugguccguggcgcguucgcuuuauuuauggcgcacauuacacggucgaccucuuugcaguaucuaauc

SEQ ID NO: 166 - RNA, 20 nt, Artificial Sequence
  Description of Artificial Sequence Synthetic oligonucleotide
agagguauagggcaugggaa

SEQ ID NO: 167 - RNA, 110 nt, Artificial Sequence; identical to SEQ ID NO: 42
  Description of Artificial Sequence Synthetic polynucleotide

SEQ ID NO: 168 - RNA, 23 nt, Artificial Sequence
  Description of Artificial Sequence Synthetic oligonucleotide
aaugacacgaucacucccguuga

SEQ ID NO: 169 - RNA, 22 nt, Artificial Sequence
  Description of Artificial Sequence Synthetic oligonucleotide
uggaauguaaagaaguauguau

SEQ ID NO: 170 - RNA, 21 nt, Artificial Sequence
  Description of Artificial Sequence Synthetic oligonucleotide
gugucuuuugcucugcaguca

SEQ ID NO: 171 - RNA, 87 nt, Artificial Sequence
  Description of Artificial Sequence Synthetic oligonucleotide
caauagacacccaucgugucuuuugcucugcagucaguaaauauuuuuuugugaauguguagcaaaagacagaauggugguccauug

SEQ ID NO: 172 - RNA, 21 nt, Artificial Sequence
  Description of Artificial Sequence Synthetic oligonucleotide
uacaguacugugauaacugaa

SEQ ID NO: 173 - RNA, 79 nt, Artificial Sequence
  Description of Artificial Sequence Synthetic oligonucleotide
acuguccuuuuucgguuaucaugguaccgaugcuguauaucugaaagguacaguacugugauaacugaagaaugguggu

SEQ ID NO: 174 - RNA, 22 nt, Artificial Sequence
  Description of Artificial Sequence Synthetic oligonucleotide
aaaccguuaccauuacugaguu

SEQ ID NO: 175 - RNA, 72 nt, Artificial Sequence
  Description of Artificial Sequence Synthetic oligonucleotide
cuugggaauggcaaggaaaccguuaccauuacugaguuuaguaaugguaaugguucucuugcuauacccaga

SEQ ID NO: 176 - RNA, 21 nt, Artificial Sequence
  Description of Artificial Sequence Synthetic oligonucleotide
cauuauuacuuuugguacgcg

SEQ ID NO: 177 - RNA, 85 nt, Artificial Sequence; identical to SEQ ID NO: 28
  Description of Artificial Sequence Synthetic oligonucleotide

SEQ ID NO: 178 - RNA, 22 nt, Artificial Sequence
  Description of Artificial Sequence Synthetic oligonucleotide
uggaauguaaagaaguauguau

SEQ ID NO: 179 - RNA, 71 nt, Artificial Sequence; identical to SEQ ID NO: 39
  Description of Artificial Sequence Synthetic oligonucleotide

SEQ ID NO: 180 - RNA, 22 nt, Artificial Sequence
  Description of Artificial Sequence Synthetic oligonucleotide
aacccguagauccgaacuugug

SEQ ID NO: 181 - RNA, 80 nt, Artificial Sequence
  Description of Artificial Sequence Synthetic oligonucleotide
ccuguugccacaaacccguagauccgaacuugugguauuaguccgcacaagcuuguaucuauagguaugugucuguuagg

SEQ ID NO: 182 - RNA, 22 nt, Artificial Sequence
  Description of Artificial Sequence Synthetic oligonucleotide
uggcagugucuuagcugguugu

SEQ ID NO: 183 - RNA, 110 nt, Artificial Sequence
  Description of Artificial Sequence Synthetic polynucleotide
ggccagcugugaguguuucuuuggcagugucuuagcugguuguugugagcaauaguaaggaagcaaucagcaaguauacugcccuagaagugcugcacguuguggggccc

SEQ ID NO: 184 - RNA, 22 nt, Artificial Sequence
  Description of Artificial Sequence Synthetic oligonucleotide
ugagguaguaguuugugcuguu

SEQ ID NO: 185 - RNA, 84 nt, Artificial Sequence; identical to SEQ ID NO: 46
  Description of Artificial Sequence Synthetic oligonucleotide

SEQ ID NO: 186 - RNA, 21 nt, Artificial Sequence
  Description of Artificial Sequence Synthetic oligonucleotide
cauuauuacuuuugguacgcg

SEQ ID NO: 187 - RNA, 85 nt, Artificial Sequence; identical to SEQ ID NO: 28
  Description of Artificial Sequence Synthetic oligonucleotide

SEQ ID NO: 188 - RNA, 23 nt, Artificial Sequence
  Description of Artificial Sequence Synthetic oligonucleotide
caaagugcucauagugcagguag

SEQ ID NO: 189 - RNA, 69 nt, Artificial Sequence
  Description of Artificial Sequence Synthetic oligonucleotide
aguaccaaagugcucauagugcagguaguuuuggcaugacucuacuguaguaugggcacuuccaguacu

SEQ ID NO: 190 - RNA, 22 nt, Artificial Sequence
  Description of Artificial Sequence Synthetic oligonucleotide
agaucagaaggugauuguggcu

SEQ ID NO: 191 - RNA, 73 nt, Artificial Sequence
  Description of Artificial Sequence Synthetic oligonucleotide
cuccucagaucagaaggugauuguggcuuuggguggauauuaaucagccacagcacugccuggucagaaagag

SEQ ID NO: 192 - RNA, 23 nt, Artificial Sequence
  Description of Artificial Sequence Synthetic oligonucleotide
aaaagugcuuacagugcagguag

SEQ ID NO: 193 - RNA, 81 nt, Artificial Sequence; identical to SEQ ID NO: 72
  Description of Artificial Sequence Synthetic oligonucleotide

SEQ ID NO: 194 - RNA, 22 nt, Artificial Sequence
  Description of Artificial Sequence Synthetic oligonucleotide
ucguaccgugaguaauaaugcg

SEQ ID NO: 195 - RNA, 85 nt, Artificial Sequence; identical to SEQ ID NO: 28
  Description of Artificial Sequence Synthetic oligonucleotide

SEQ ID NO: 196 - RNA, 97 nt, Artificial Sequence
  Description of Artificial Sequence Synthetic oligonucleotide
acucaggggcuucgccacugauuguccaaacgcaauucuuguacgagucugcggccaaccgagaauuguggcuggacaucuguggcugagcuccggg

SEQ ID NO: 197 - RNA, 22 nt, Artificial Sequence
  Description of Artificial Sequence Synthetic oligonucleotide
ucguaccgugaguaauaaugcg

SEQ ID NO: 198 - RNA, 85 nt, Artificial Sequence; identical to SEQ ID NO: 28
  Description of Artificial Sequence Synthetic oligonucleotide

SEQ ID NO: 199 - RNA, 23 nt, Artificial Sequence
  Description of Artificial Sequence Synthetic oligonucleotide
agcagcauuguacagggcuauca

SEQ ID NO: 200 - RNA, 81 nt, Artificial Sequence; identical to SEQ ID NO: 82
  Description of Artificial Sequence Synthetic oligonucleotide

SEQ ID NO: 201 - RNA, 21 nt, Artificial Sequence
  Description of Artificial Sequence Synthetic oligonucleotide
uaaagugcugacagugcagau

SEQ ID NO: 202 - RNA, 82 nt, Artificial Sequence; identical to SEQ ID NO: 162
  Description of Artificial Sequence Synthetic oligonucleotide

SEQ ID NO: 203 - RNA, 22 nt, Artificial Sequence
  Description of Artificial Sequence Synthetic oligonucleotide
uagcuuaucagacugauguuga

SEQ ID NO: 204 - RNA, 72 nt, Artificial Sequence; identical to SEQ ID NO: 8
  Description of Artificial Sequence Synthetic oligonucleotide

SEQ ID NO: 205 - RNA, 22 nt, Artificial Sequence
  Description of Artificial Sequence Synthetic oligonucleotide
aucgugcaucccuuuagagugu

SEQ ID NO: 206 - RNA, 87 nt, Artificial Sequence; identical to SEQ ID NO: 119
  Description of Artificial Sequence Synthetic oligonucleotide

(19) United States
(12) Patent Application Publication
(10) Pub. No.: US 2010/0323357 A1
(43) Pub. Date: Dec. 23, 2010

(54) MicroRNA Expression Profiling and Targeting in Peripheral Blood in Lung Cancer

(75) Inventors: Serge P. Nana-Sinkam, Columbus, OH (US); Clay B. Marsh, Columbus, OH (US); Melissa G. Piper, Dublin, OH (US); Gregory A. Otterson, Upper Arlington, OH (US)

Correspondence Address: MACMILLAN SOBANSKI & TODD, LLC, ONE MARITIME PLAZA FIFTH FLOOR, 720 WATER STREET, TOLEDO, OH, 43604-1619, US

(73) Assignee: THE OHIO STATE UNIVERSITY RESEARCH FOUNDATION, Columbus, OH (US)

(21) Appl. No.: 12/745,327

(22) PCT Filed: Nov. 26, 2008
(86) PCT No.: PCT/US08/84821

§ 371 Date: Jul. 8, 2010

Related U.S. Application Data
(60) Provisional application No. 61/004,863, filed on Nov. 30, 2007.

(51) Int. Cl. C12Q 1/68

(52) U.S. Cl. 435/6

(57) ABSTRACT

A method for the diagnosis, prognosis and treatment of lung cancer by detecting at least one microRNA in peripheral blood is disclosed.

### CROSS-REFERENCE TO RELATED APPLICATIONS AND STATEMENT REGARDING FEDERALLY SPONSORED RESEARCH

This application claims the benefit of U.S. Provisional Application No. 61/004,863, filed Nov. 30, 2007, the disclosure of which is incorporated herein by reference. This invention was made with no Government support and the Government has no rights in this invention.

### TECHNICAL FIELD AND INDUSTRIAL APPLICABILITY OF THE INVENTION

This invention is directed to certain methods for the diagnosis, prognosis and treatment of lung cancer by detecting at least one microRNA (miR) in peripheral blood

### BACKGROUND OF THE INVENTION

Lung cancer is the leading cause of cancer death in men and women in the United States with a dismal 5-year survival rate of <15%. In the last several years, epidemiologic statistics reveal that the majority of lung cancers are diagnosed in former smokers and never smokers.

Although there has been a slight decrease in cases and mortality from lung cancer in recent years, in 2008, 215,020 new cases and 161,840 deaths are estimated. There are no established screening tests for early detection of lung cancer, and less than 25% of subjects present with surgically curable disease (stages I and II). In addition, while the five year survival of early resectable disease approaches 70-80%, recurrence of disease remains unacceptably high.

It is increasingly recognized that lung cancer represents a group of heterogeneous diseases that, despite similar morphology, exhibit different growth rates, metastatic potential and response to therapies. Given the high incidence of lung cancer among former smokers, risk stratification and identification of early treatable disease is of great importance.

Clinically and pathologically, lung cancer is broadly divided into two distinct categories, small cell lung cancer (SCLC) and non-small cell lung cancer (NSCLC). SCLC represents approximately 20% of all lung cancer and is characterized by a rapid growth rate and widespread disease on initial diagnosis. NSCLC (accounting for 80% of lung cancers) is a collection of at least three distinct pathological entities (adeno-carcinoma, squamous cell carcinoma and large cell carcinoma) that behave and are treated clinically in a similar fashion. NSCLC tends to be more indolent than SCLC and is less responsive to chemotherapy. The mainstay of treatment for SCLC is chemotherapy plus or minus radiation therapy, whereas the primary treatment modality for NSCLC is surgery with the judicious addition of radiation and/or chemotherapy.

MicroRNAs (MiRNAs, miRs) are a family of small non-coding RNAs (approximately 21-25 nt long) expressed in many organisms including animals, plants, and viruses. MiRNAs target genes for either degradation of mRNA or inhibition of protein translation. A single miRNA may target multiple genes while a single gene may be targeted by multiple miRNAs. Although the function of most miRNAs remains unknown, several studies suggest that they may be integral to key biological functions including gene regulation, apoptosis, hematopoietic development and the maintenance of cell differentiation. It is estimated that greater than 50% of miRNAs are located in chromosomal regions that are known to be either deleted or amplified in cancer.

Knowledge of miRNAs in lung cancer is starting to emerge. Previously, investigators identified that multiple miR-let-7 family genes can target the 3′-untranslated region (UTR) of nematode RAS gene (let-60) in C. elegans. Over-expression of let-7 inhibits the expression of RAS protein and let-7 complementary sites are seen in human NRAS and KRAS 3′-UTR. RAS signaling is believed to help initiate the deletions of human let-7 genomic regions in lung cancer. Indeed, reduced let-7 expression in 143 resected lung cancer cases correlated with worse prognosis.

Recently, through the use of miRNA chip analysis, investigators demonstrated distinct miRNA profiles in 104 pairs of primary lung cancers and corresponding non-cancerous tissue. In addition, five distinct miRNAs (miR-155, 17-3p, let-7a-2, 145 and 21) were altered in expression and predicted prognosis among subjects with adenocarcinoma.

Genomic platforms have become powerful tools in identifying histological subcategories of disease, new molecular targets, prognostic tools and response to therapies. However, while there is improvement in the reproducibility of studies in lung cancer, there remains variability in histological classifications utilizing microarray analysis. In addition, genomic studies do not address the lack of validation in gene expression nor biological relevance. A systems approach of integrating several platforms of analysis may be required to better clarify the molecular heterogeneity in lung cancer.

For microarray studies to correctly identify diagnostic markers and therapeutic targets, however, it is necessary to determine which changes in gene expression are validated by protein analysis. Furthermore, the development of functional readouts is necessary in order to determine the biological significance of microarray analysis.

There is is now presented herein a signature set of specific biomarkers that can be used in the genomic analysis of peripheral blood that is useful to discriminate lung cancer subjects from normal controls.

### SUMMARY OF THE INVENTION

In a first broad aspect, there is provided a method of diagnosing whether a subject has, or is at risk for developing, lung cancer. The method includes measuring the level of at least one miR gene product in peripheral blood test sample from the subject. An alteration in the level of the miR gene product in the test sample, relative to the level of a corresponding miR gene product in a control sample, is indicative of the subject either having, or being at risk for developing, lung cancer.

In another aspect, there is provided herein a method using gene expression patterns in the peripheral blood as a noninvasive biomarker for disease diagnosis and prognosis.

In another broad aspect, there is provided herein a method of determining the prognosis of a subject with one or more lung cancer associated diseases.

Various objects and advantages of this invention will become apparent to those skilled in the art from the following detailed description of the preferred embodiment, when read in light of the accompanying drawings.

### BRIEF DESCRIPTION OF THE DRAWINGS

FIG. 1—Tile representing whole peripheral blood miRNAs from subjects with advanced lung cancer (Tumor n=4) and normal controls (Normal N=3). Green represents relatively high expression and red, relatively low expression.

FIG. 2—RT-PCR of MiR-126 expression in whole peripheral blood normal subjects (n=3) and subjects with advanced non-treated lung cancer (n=4) (*p<0.05).

FIGS. 3A-D—In situ hybridization of miR-155 in human lung cancer:

FIG. 3A—Premature MiR-155 localizes to the nucleus of Adenocarcinoma (arrows).

FIG. 3B—No detectable expression of mature form of miR-155 in same adenocarcinoma sample suggestive of impaired processing.

FIG. 3C—Premature-miR-155 in nucleus of Bronchoalveolar Cell carcinoma (BAC) (arrow).

FIG. 3D—Mature miR-155 localized to the cytoplasm.

FIGS. 4A-4D—MiR-126 transfection alters Crk protein expression:

FIG. 4A: PremiR-126 transfection of H1703 (non-small cell carcinoma) cells resulted in a 1000- to 5000-fold increase in miR-126 mRNA expression and a decrease in Crk II protein.

FIGS. 4B-4D: With no change in Crk mRNA (FIG. 4B). Crk I protein was not detectable by Western. Transfection of H226 cells with 100 nM of LNA miR-126 anti-sense oligonucleotide resulted in a 10-fold decrease in miR-126 expression compared to scrambled pre-miR transfection (FIG. 4C) and increase in Crk II protein expression as measured by densitometry (*p<0.05) but no change in mRNA (FIG. 4D). Western blots were conducted in duplicate and all RT-PCR results represent average=/−S.E. from two independent experiments conducted in duplicate. (*p<0.05 scrambled versus pre-miR) 18S was used as an internal control.

FIGS. 5A-5G: Representative images demonstrating in situ hybridization for miR-126 and immunohistochemistry for Crk in human squamous cell carcinomas of the lung. In case one, Crk expression (red) is evident within most tumor cells (FIG. 5A) while there is a lack of miR-126 expression in the adjacent section in the tumor cells (FIG. 5B) whereas miR-126 was detected in normal bronchial epithelium (FIG. 5C) (blue signal). In case number two, there is no detectable Crk within the tumor (FIG. 5D) while there is strong expression of miR-126 (blue) within the tumor (FIG. 5E); Crk localized to the endothelium (FIG. 5F); and to the bronchial epithelium (FIG. 5G) in normal tissue (All images are at 400× with the exception of FIG. 5F which is 1000× and FIG. 5G which is 200×).

FIG. 6—Graph showing relative expression of miR-126 in lung cancer (N=5) relative to normal (N=5) levels in Peripheral Blood Mononuclear Cells (PBMC). (p<0.05)

FIG. 7—Graph showing relative expression of miR-let 7a in lung cancer (N=5) relative to normal (N=5) levels in Serum. (p<0.05)

FIG. 8—Graph showing relative expression of miR-126 in lung cancer (N=5) relative to normal (N=5) levels in Serum. (p<0.05).

FIGS. 9A-9D: Effects of miR-126 over-expression on H1703 proliferation, adhesion, migration and invasion.

FIG. 9A: Control, scrambled pre-miR and pre-miR 126 cells exhibited similar rates of growth over 96 h. Two independent proliferation assays were conducted in triplicate.

FIGS. 92B-92D: MiR-126 over-expressing cells demonstrated decreased adherence (FIG. 9B), migration (FIG. 9C) and invasion (FIG. 9D). Images in FIG. 9C and FIG. 9D are representative of blinded random fields (p<0.05). In all experiments, miR-126 over-expression was confirmed by RT-PCR to ensure adequate induction. Results represent average of four fields conducted in triplicate (*p<0.05 scrambled versus pre-miR).

FIGS. 10A-10B: miR-126 and Crk expression in NSCLC tissues: Examination of 19 pairs of human non-small cell lung cancers and uninvolved adjacent lung (squamous 1-13 and adenocarcinoma 14-19) demonstrate a decrease in miR-126 mRNA expression in tumors (T) compared to uninvolved adjacent normal (N) lung (FIG. 10A). Crk mRNA expression in these same samples was variable with seven out of 19 tumors exhibiting higher Crk expression than uninvolved adjacent lung. (FIG. 10B) RT-PCR results represent average=/−S.E. from two independent experiments conducted in duplicate (*p<0.05 tumor versus uninvolved lung). 18S was used as the endogenous control.

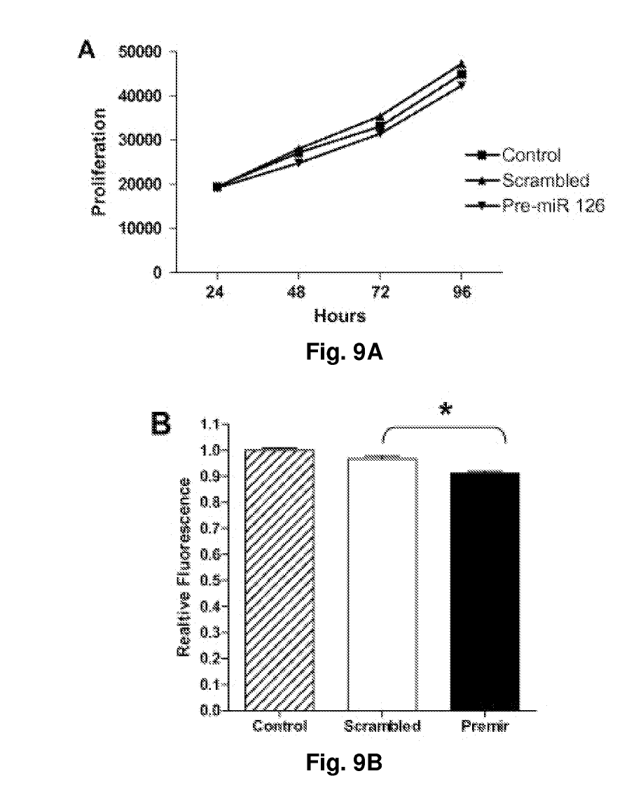

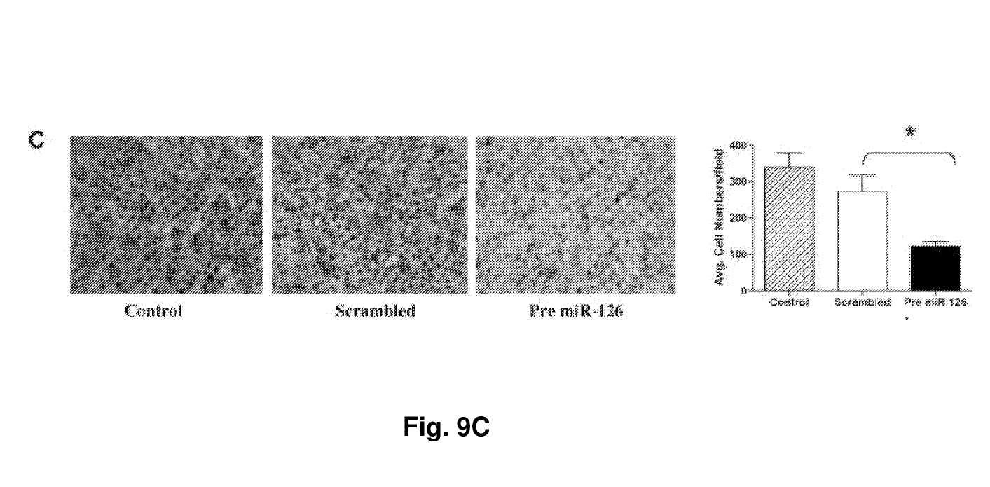

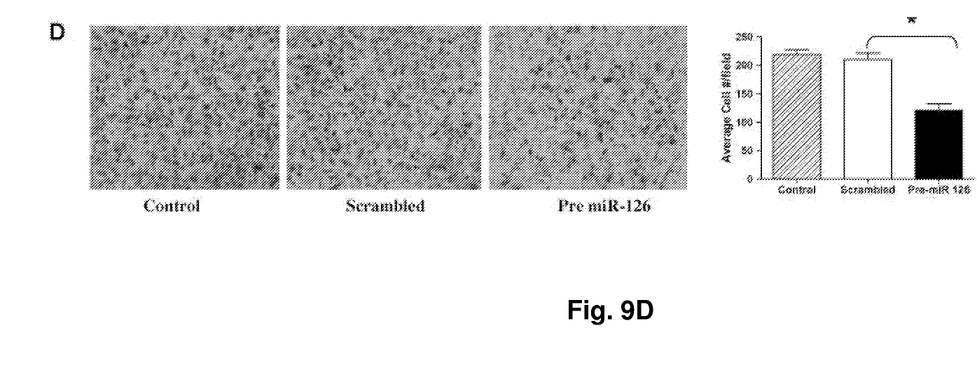

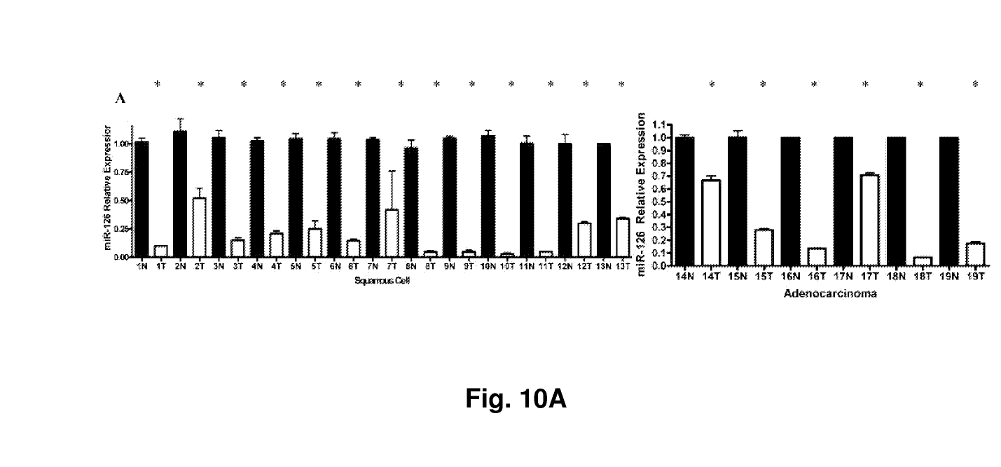

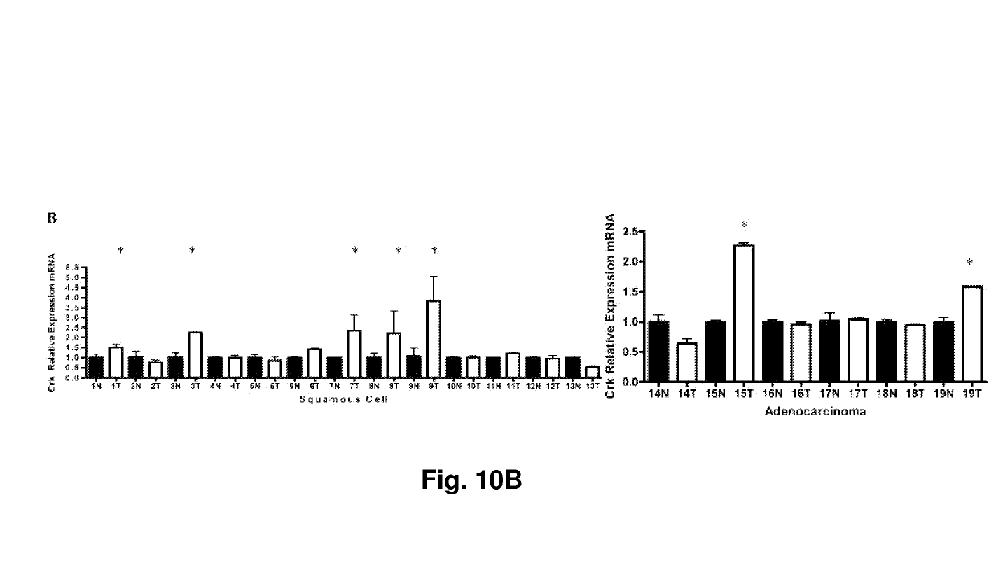

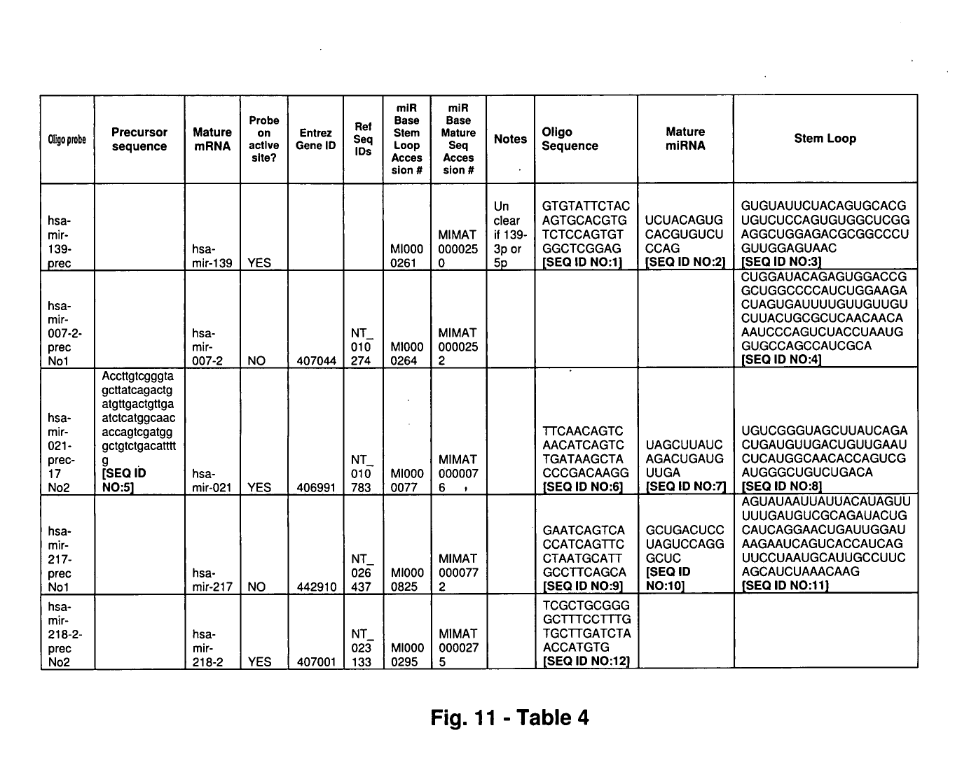

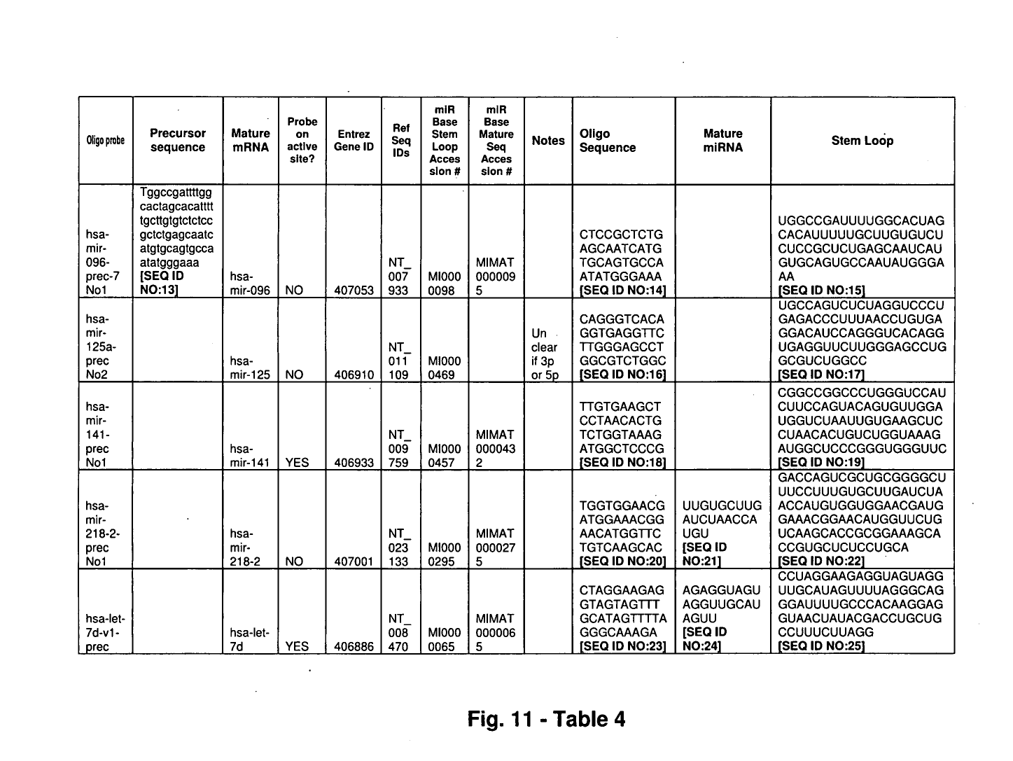

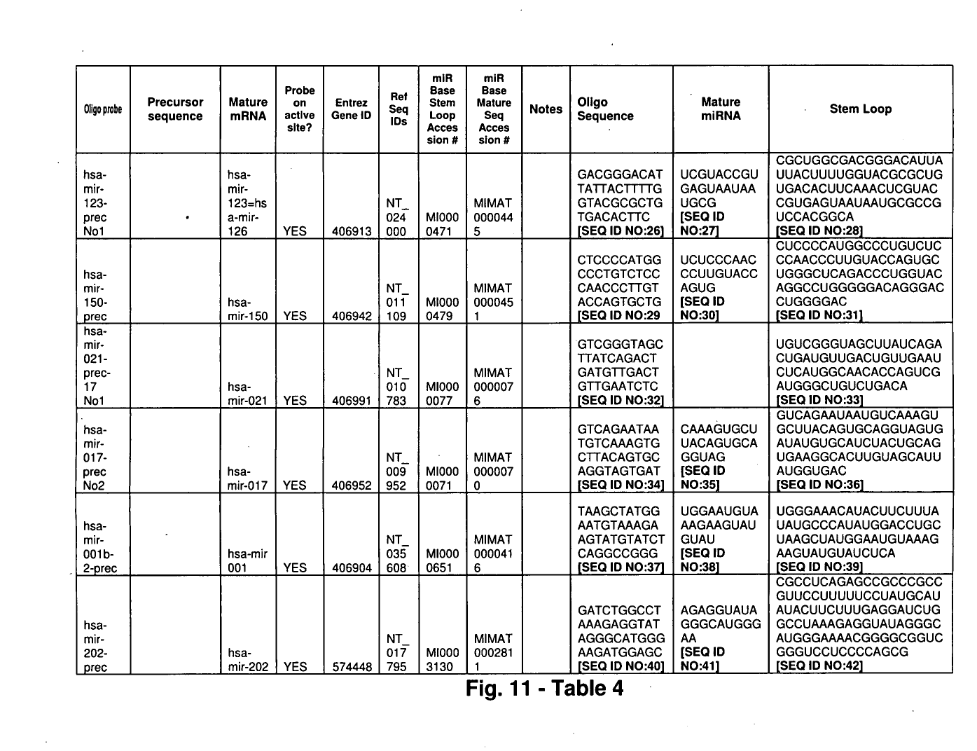

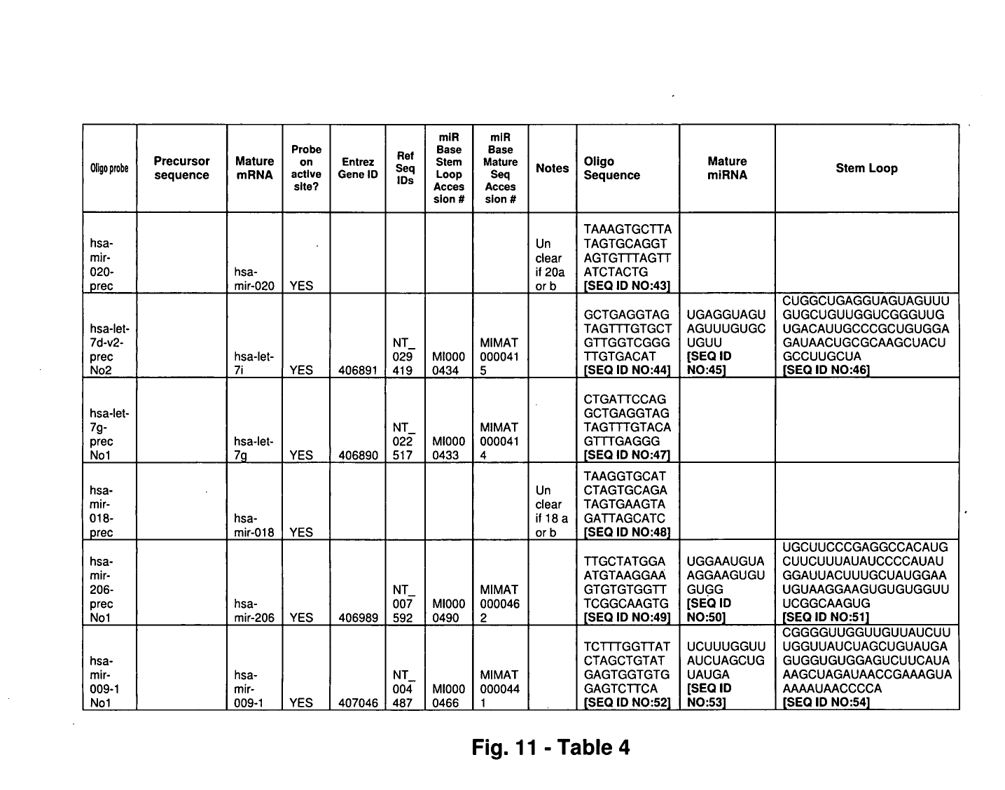

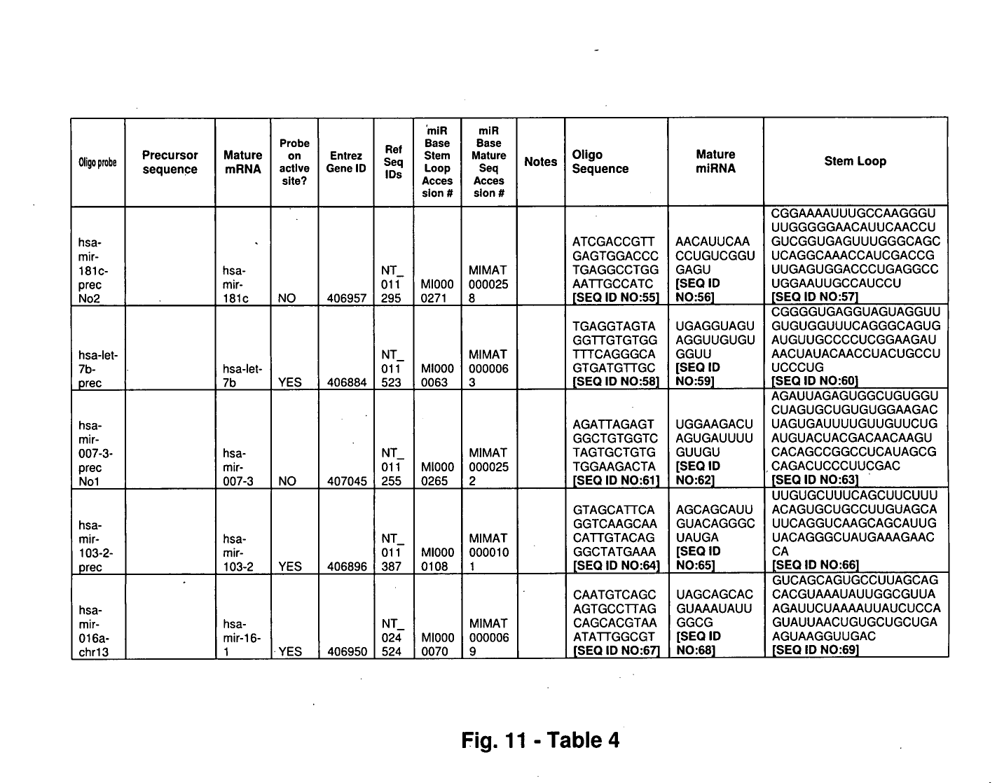

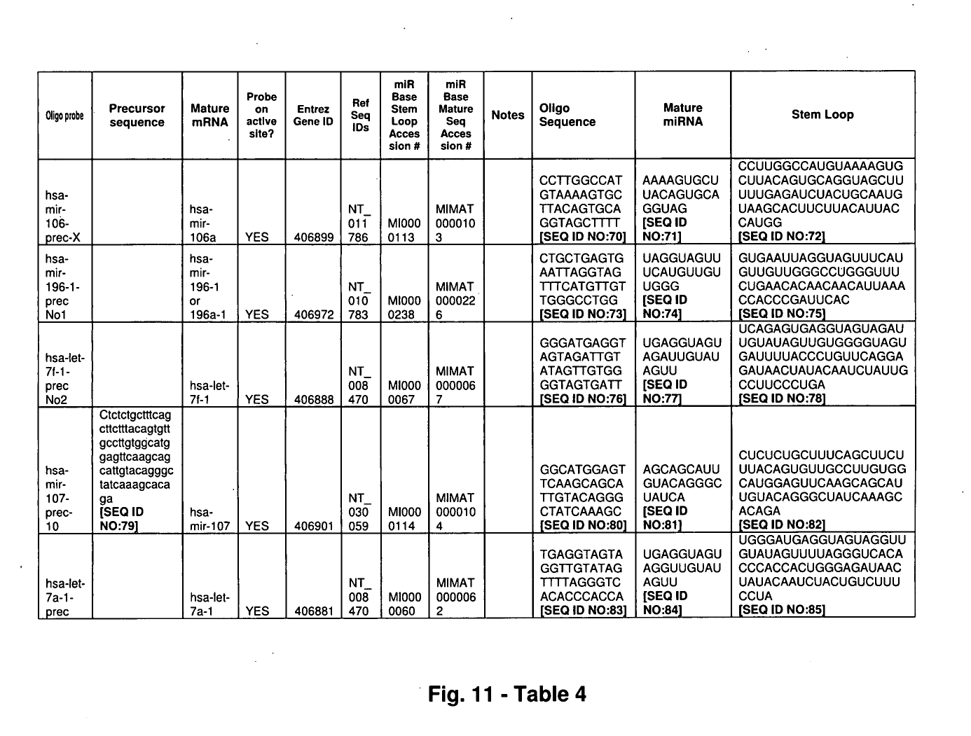

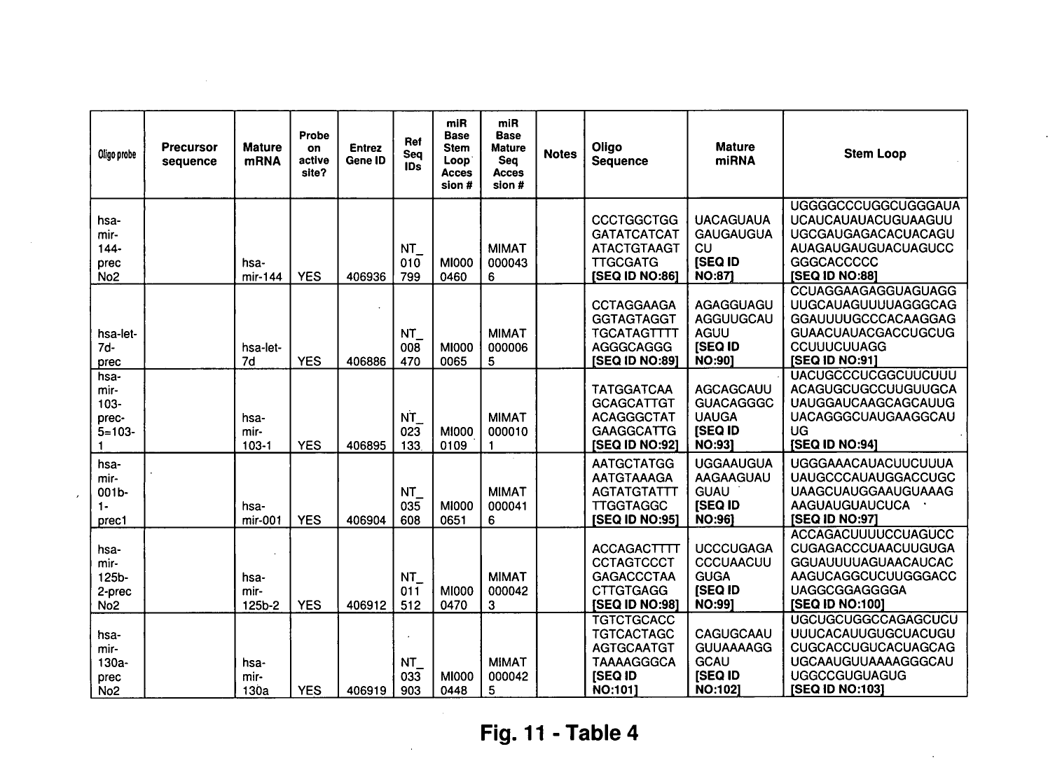

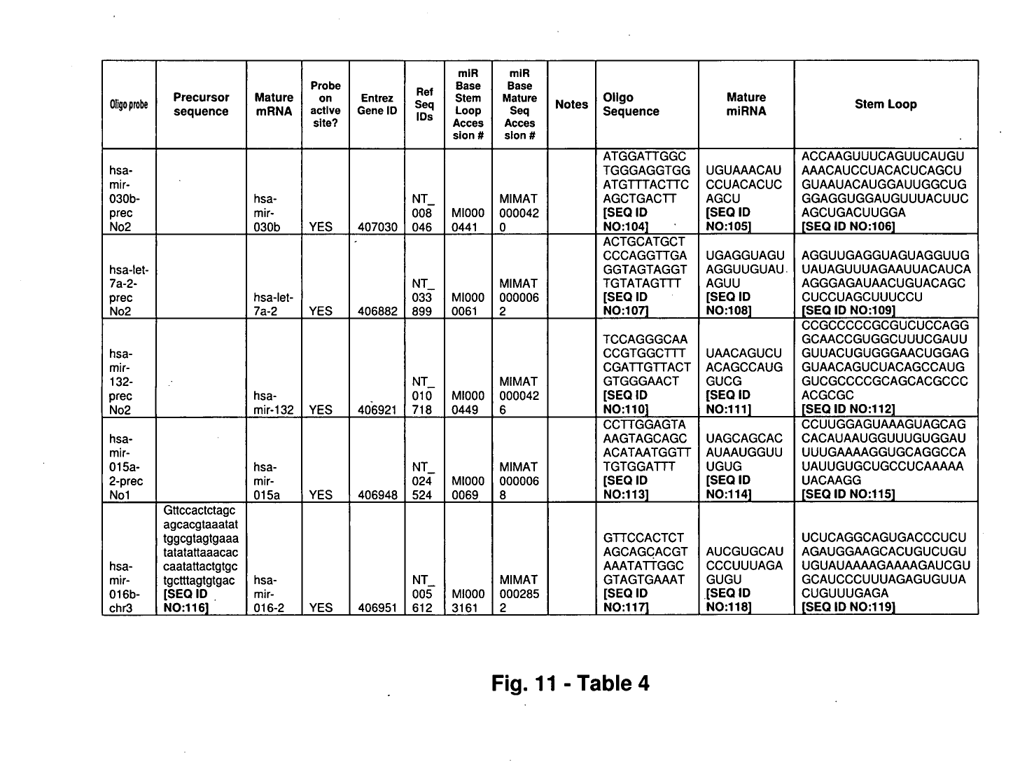

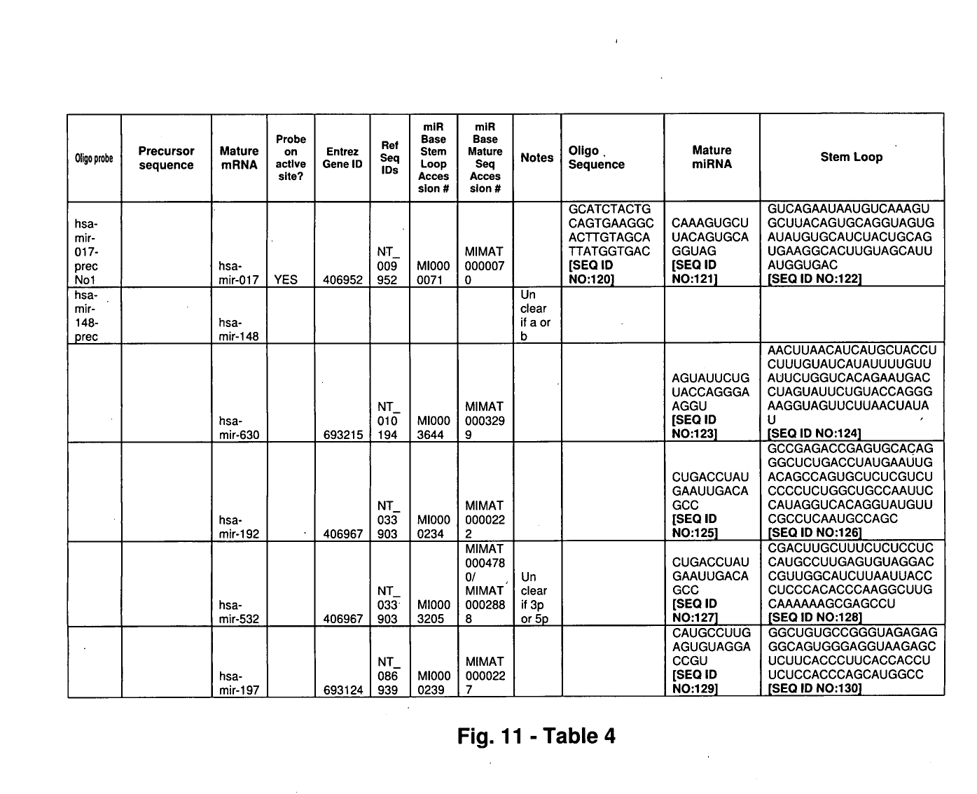

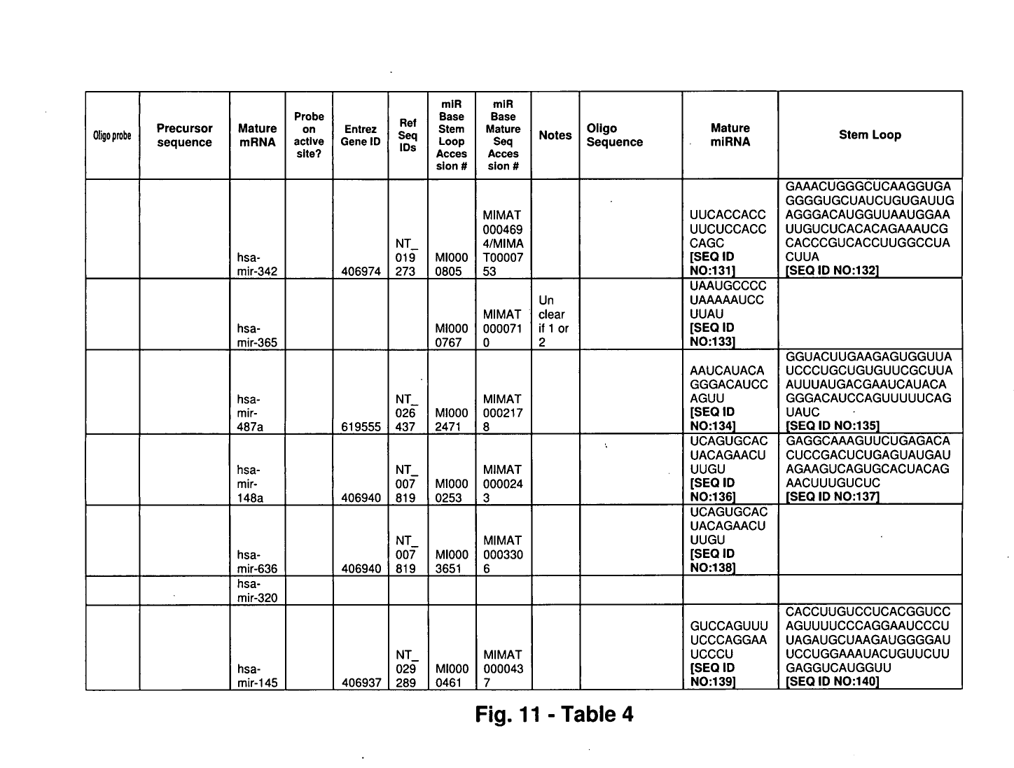

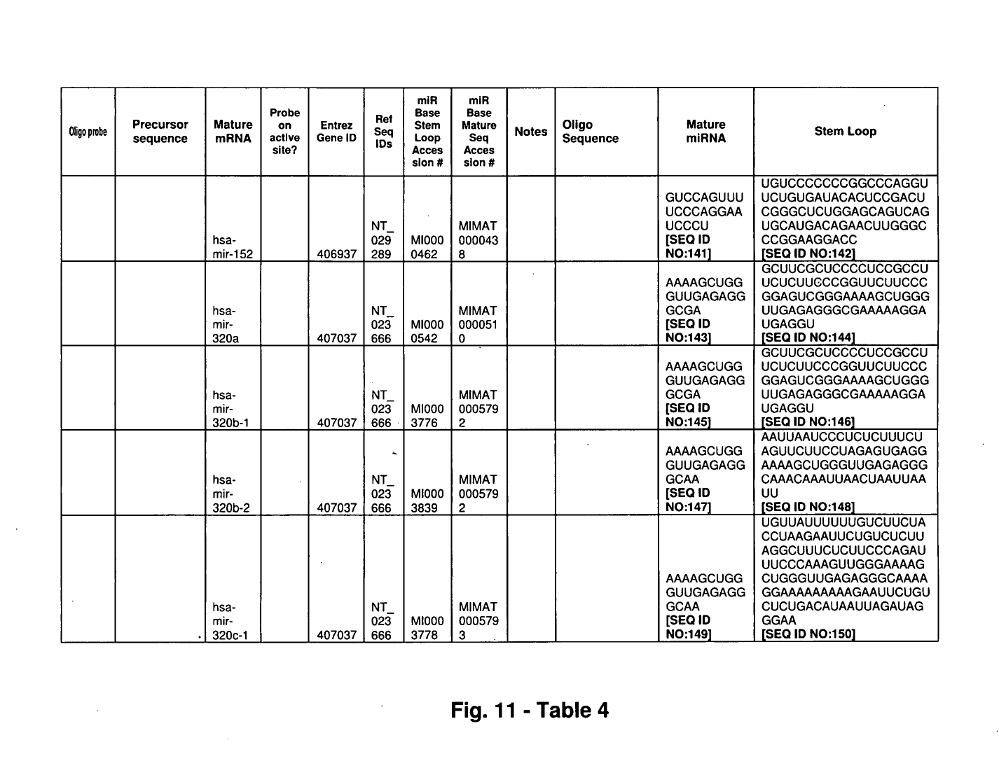

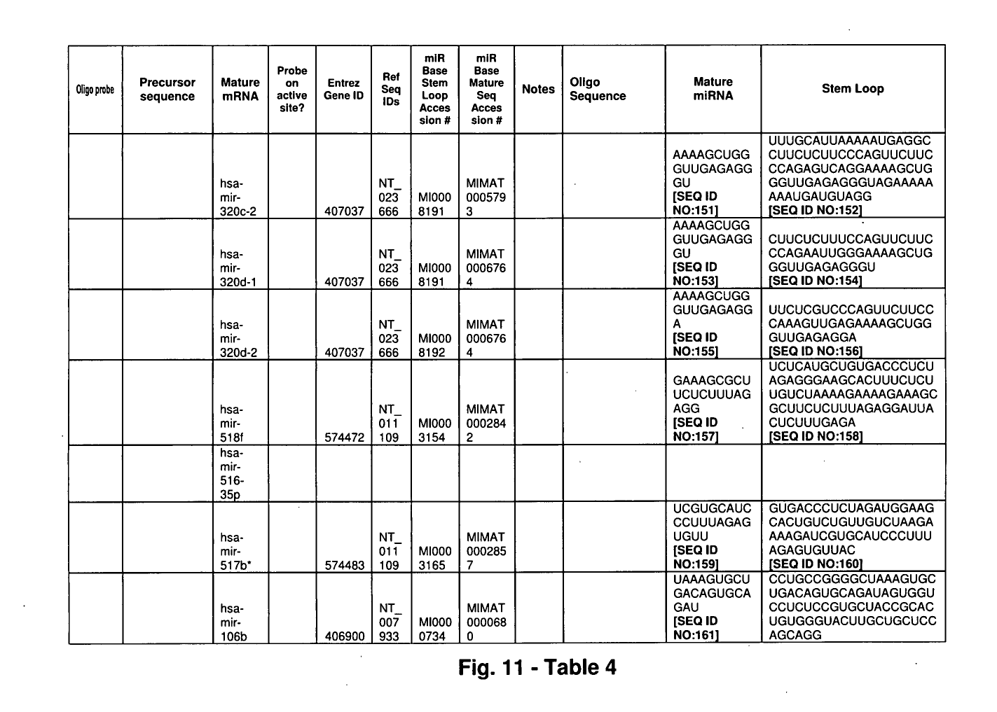

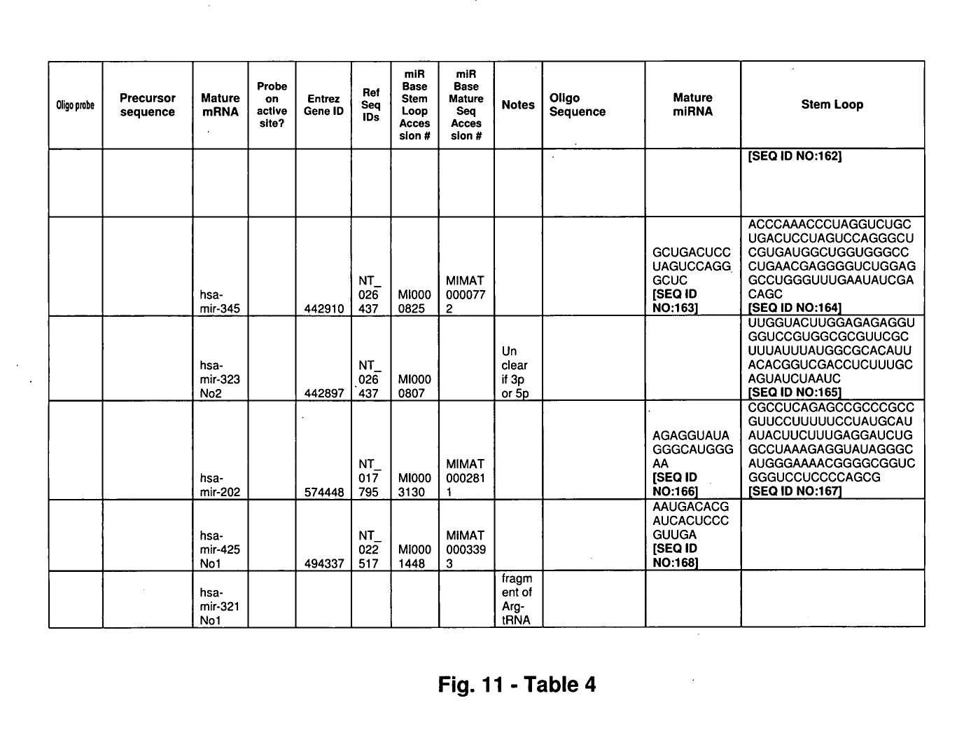

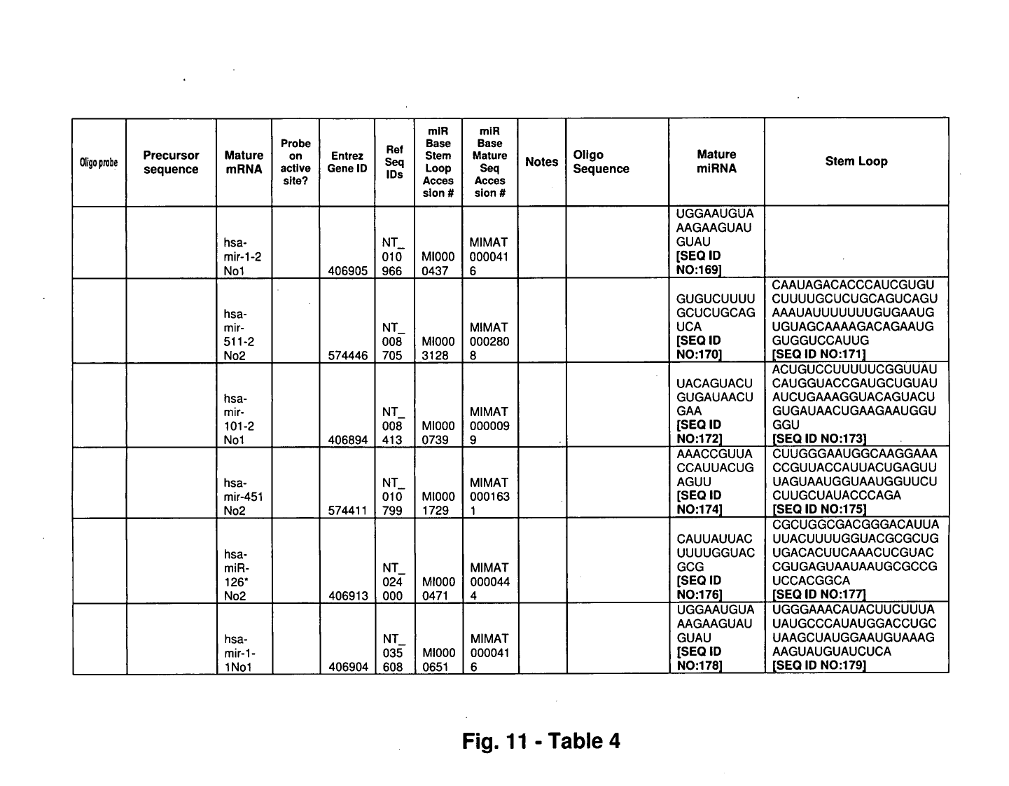

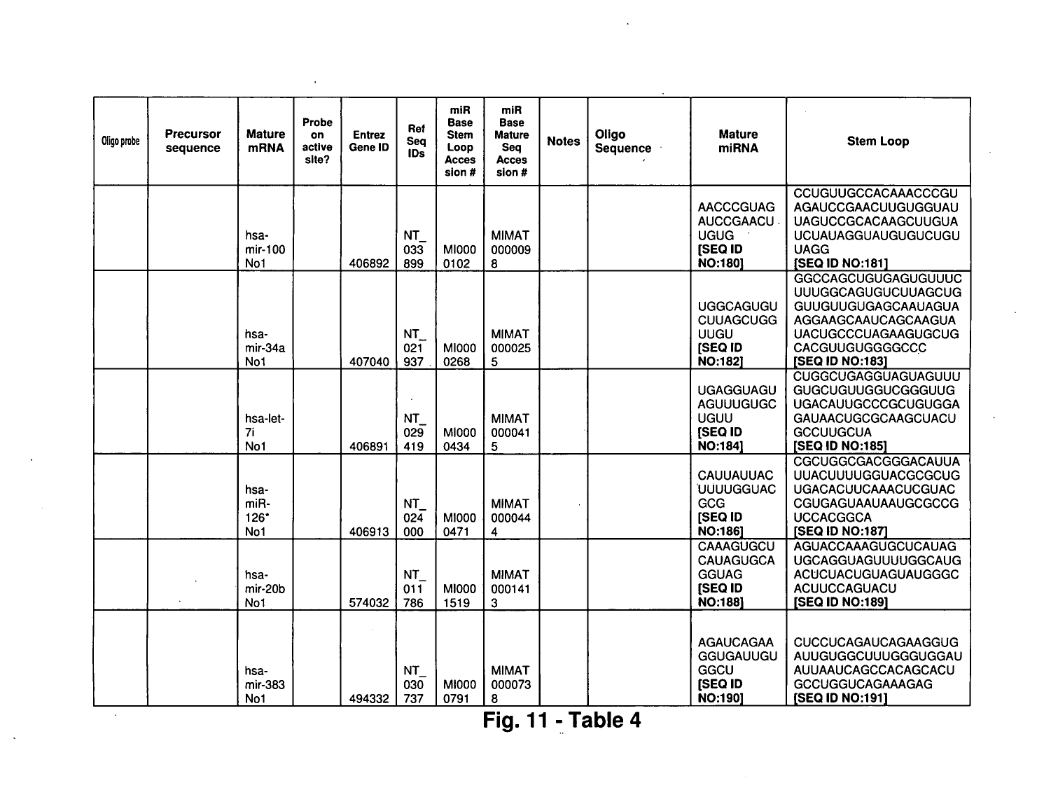

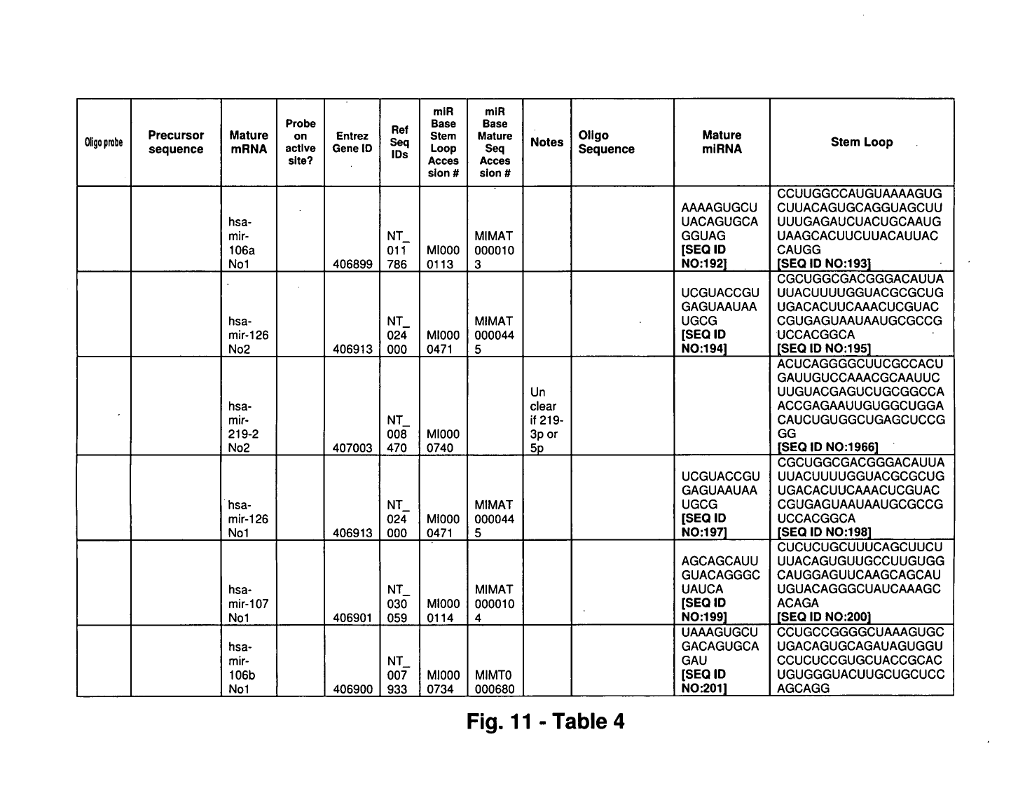

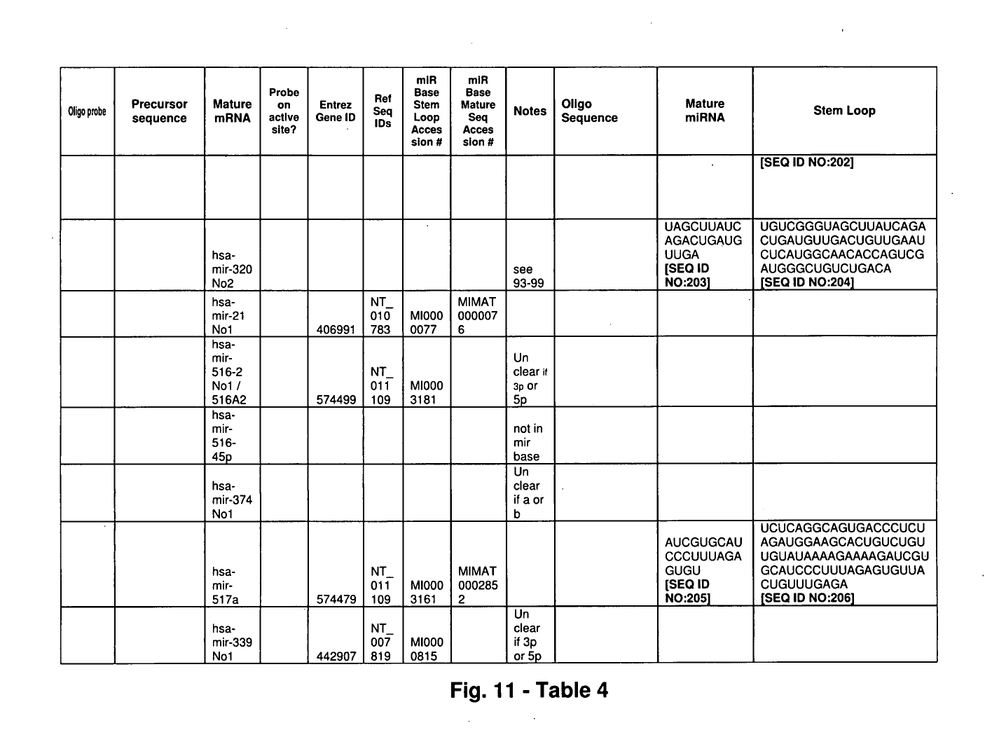

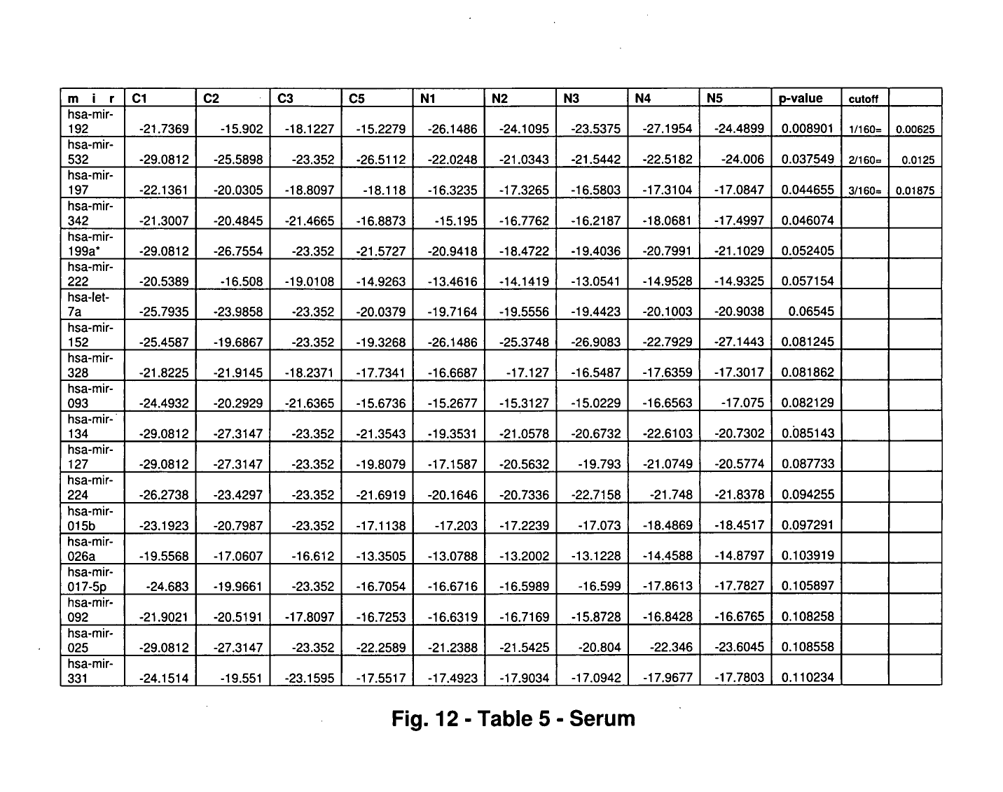

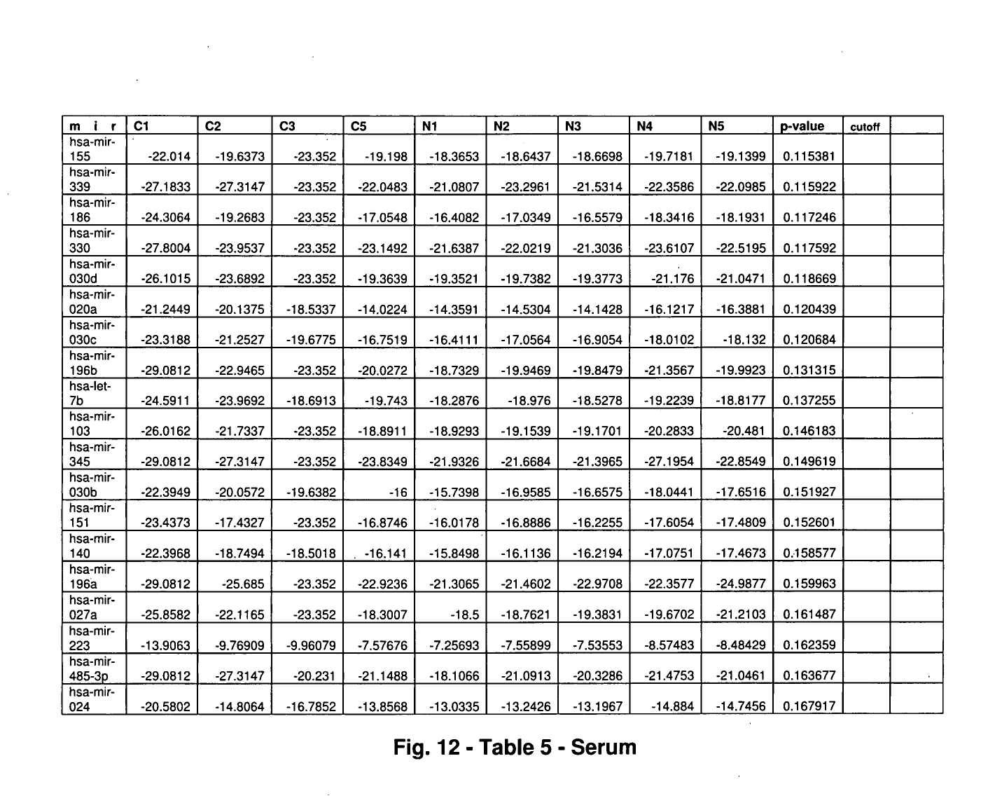

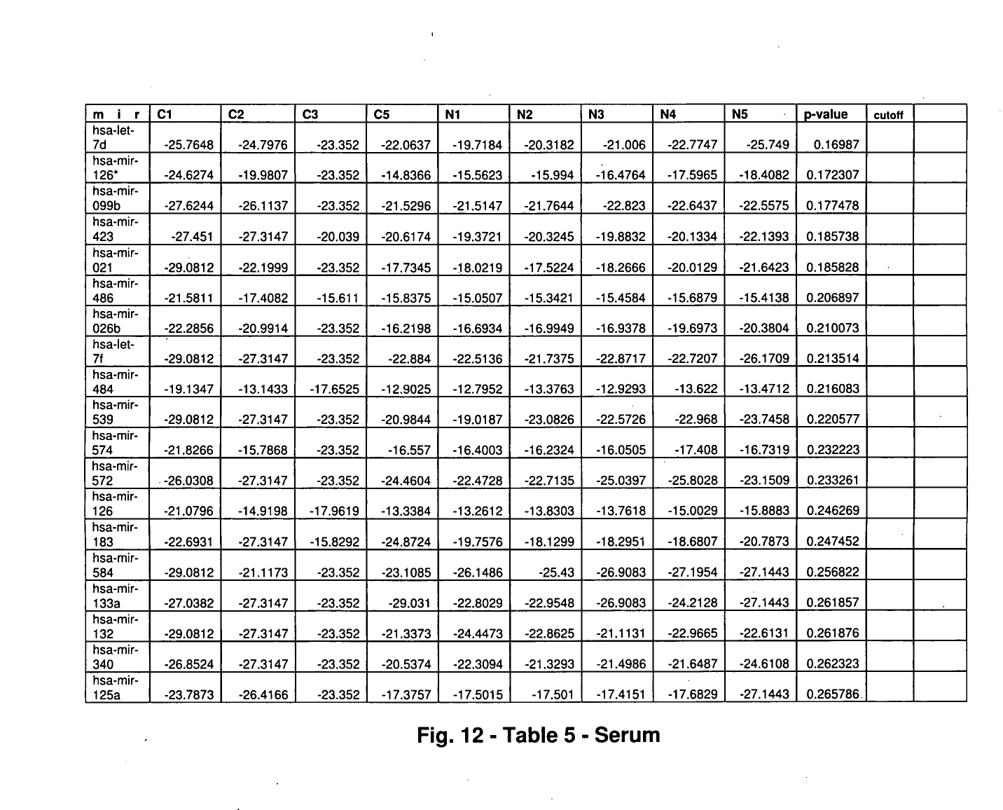

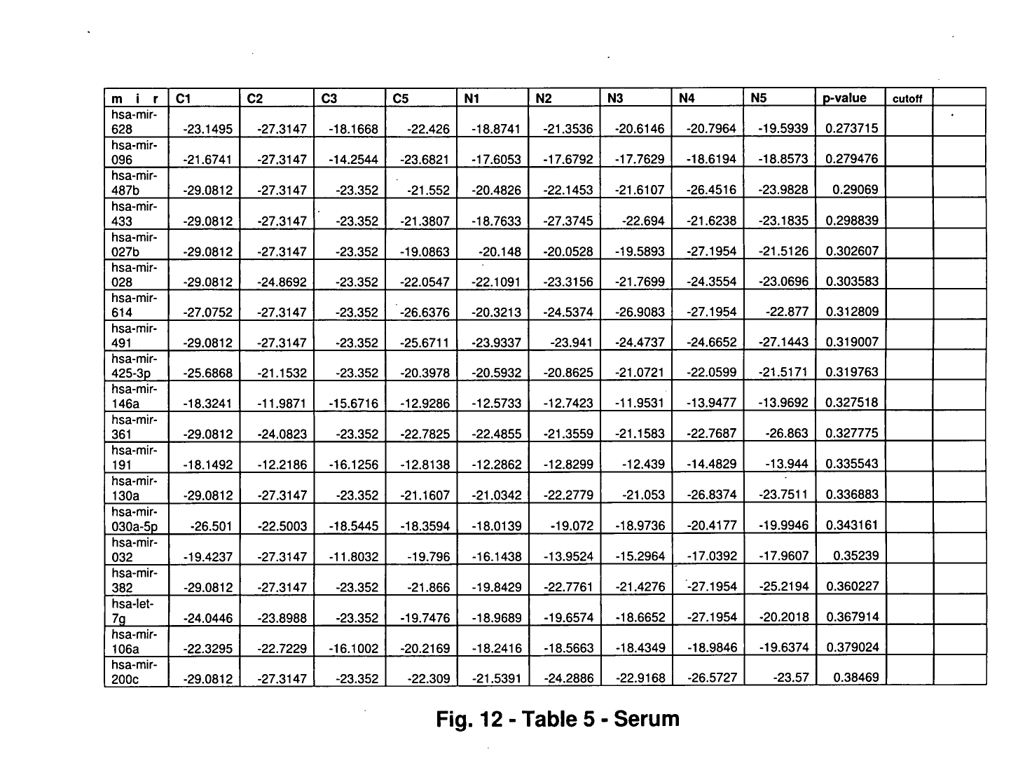

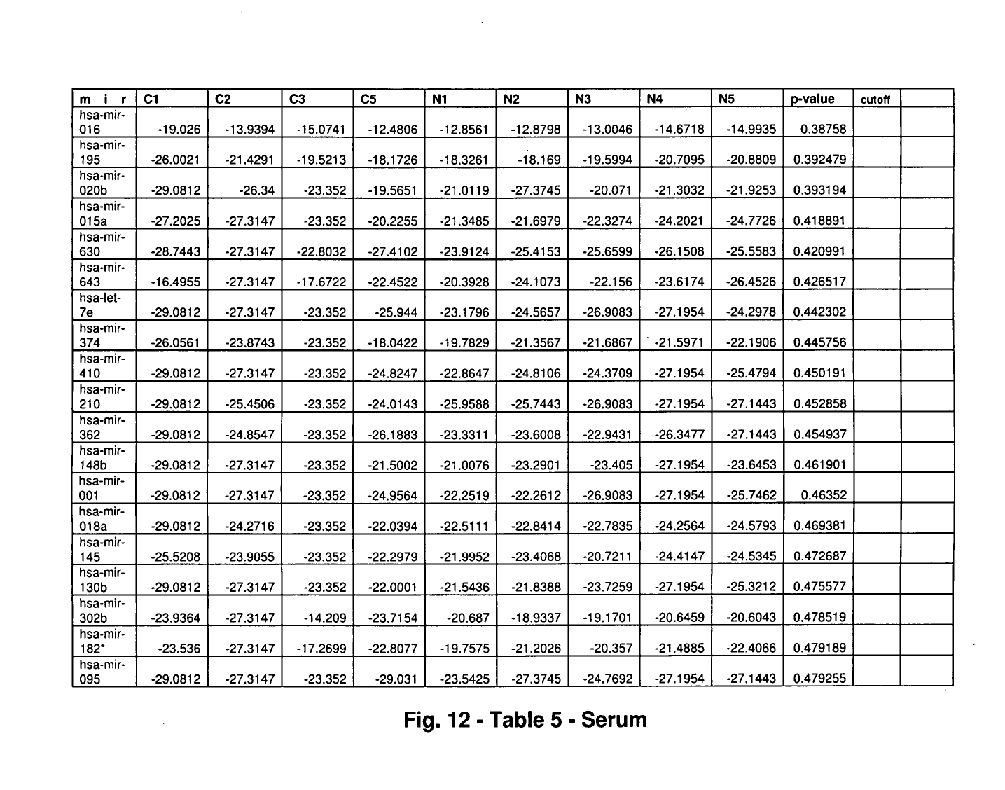

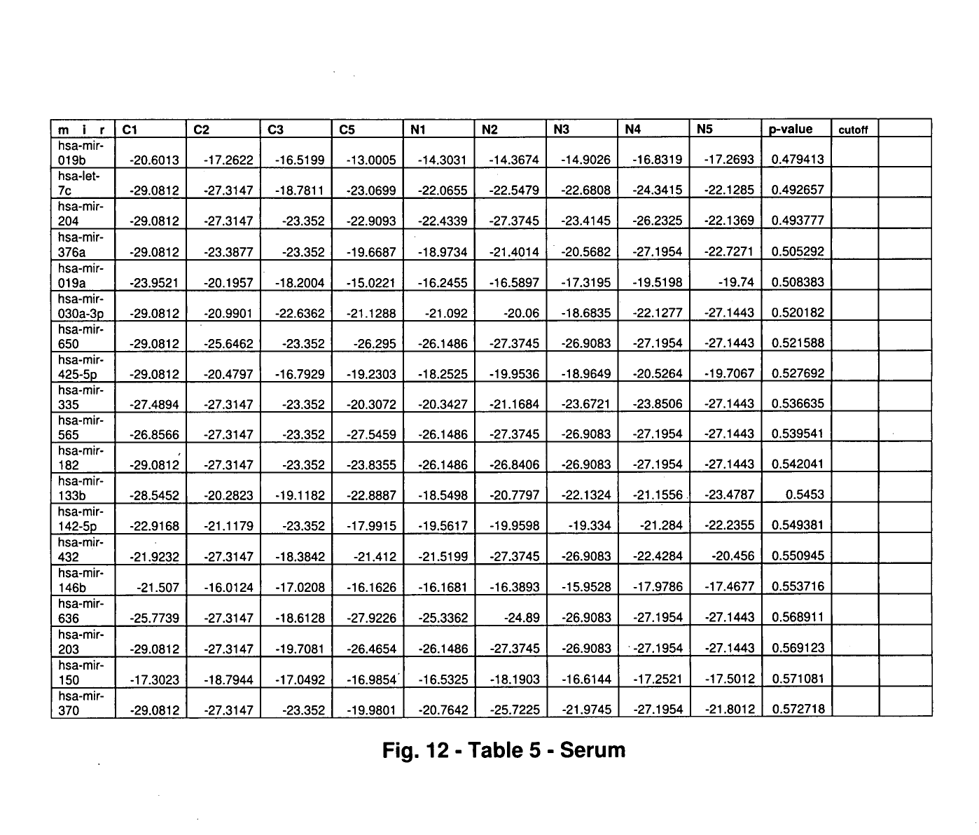

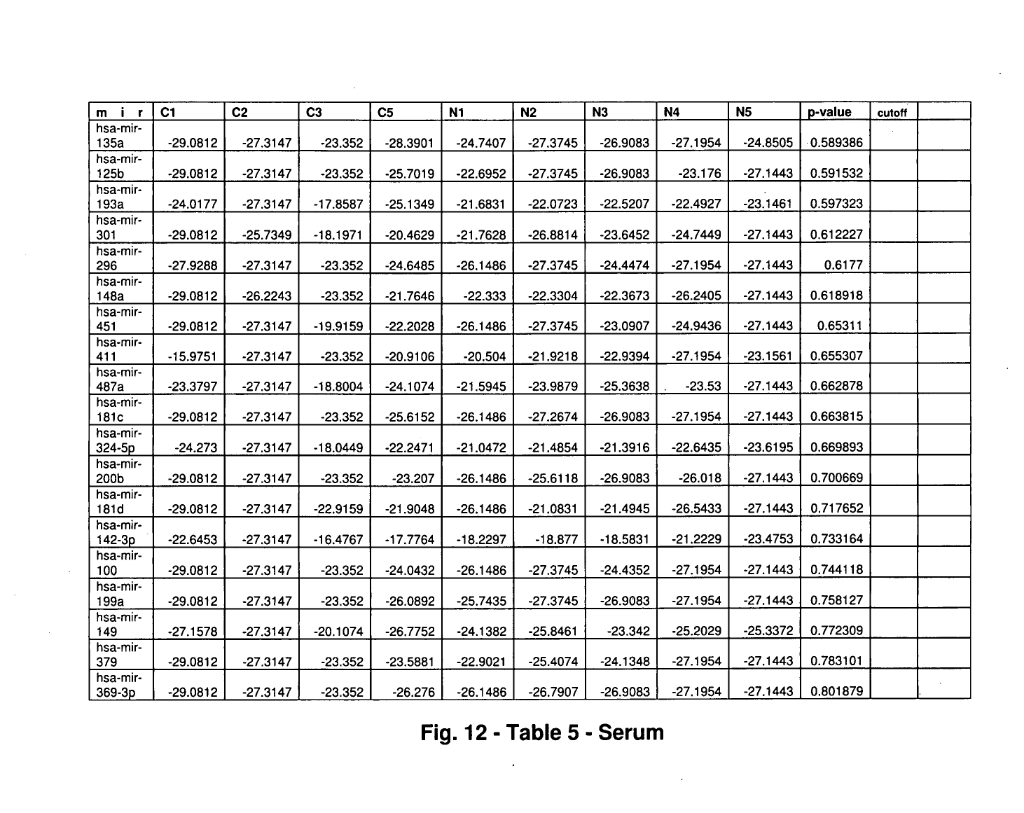

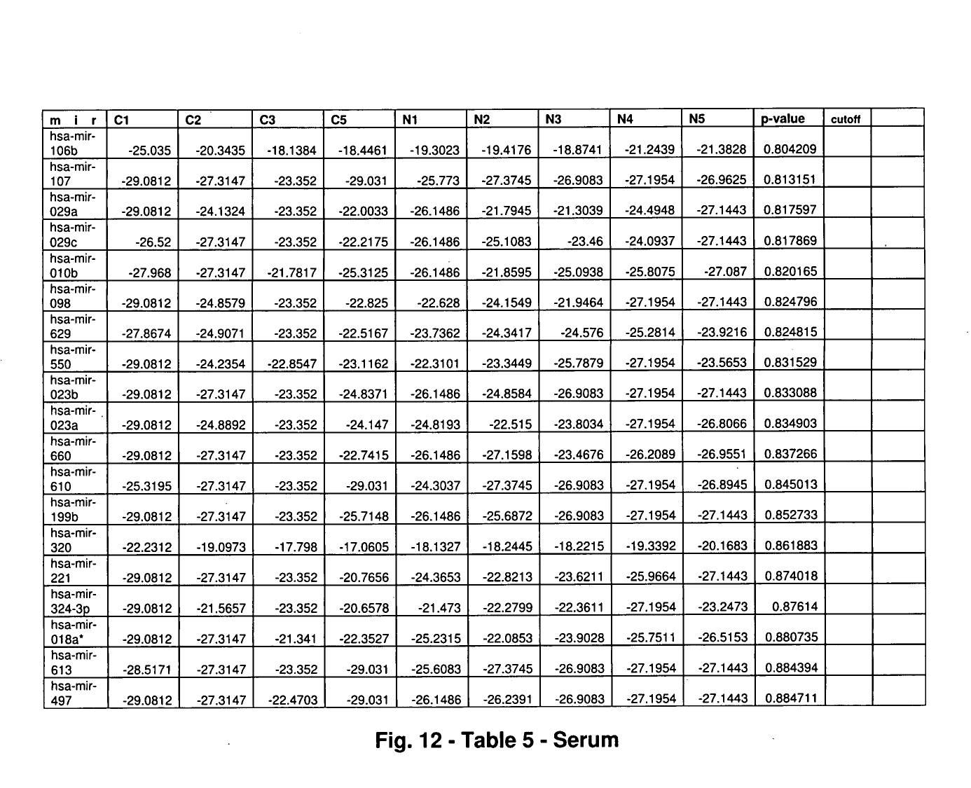

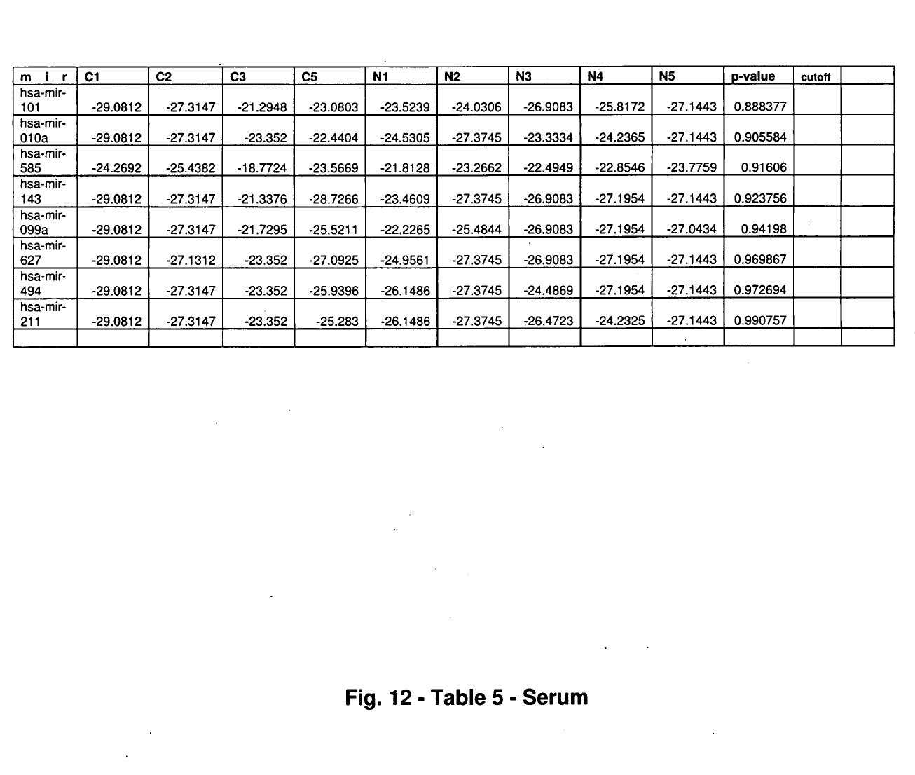

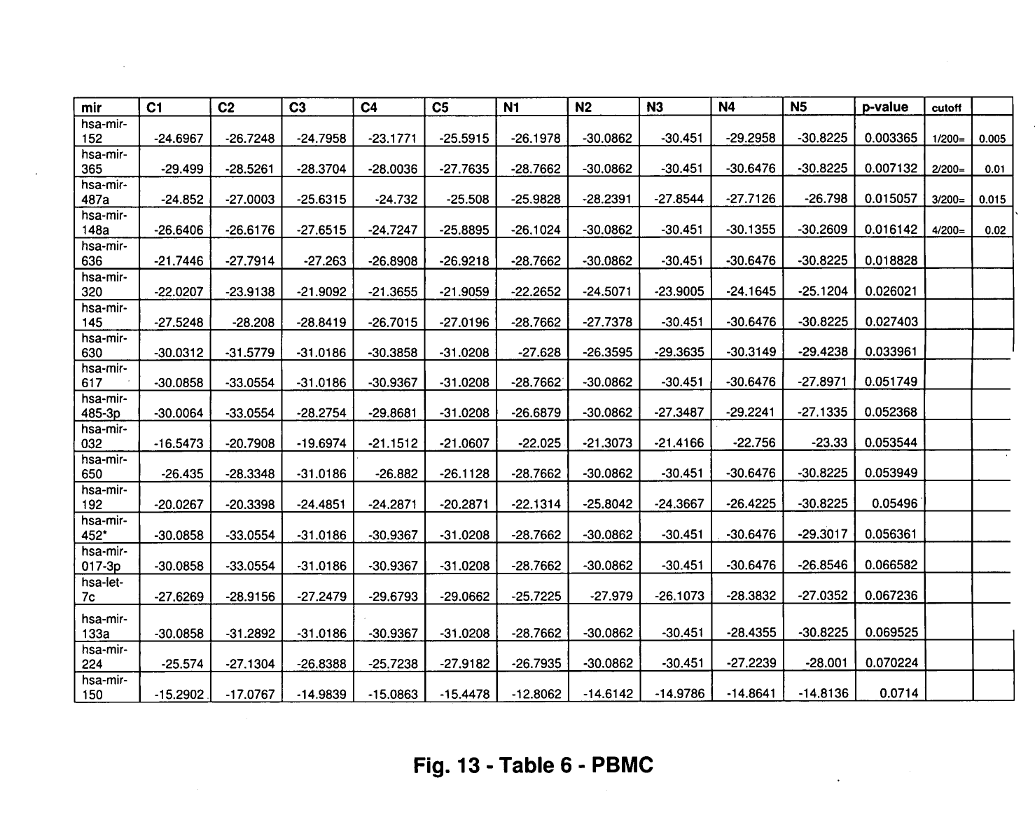

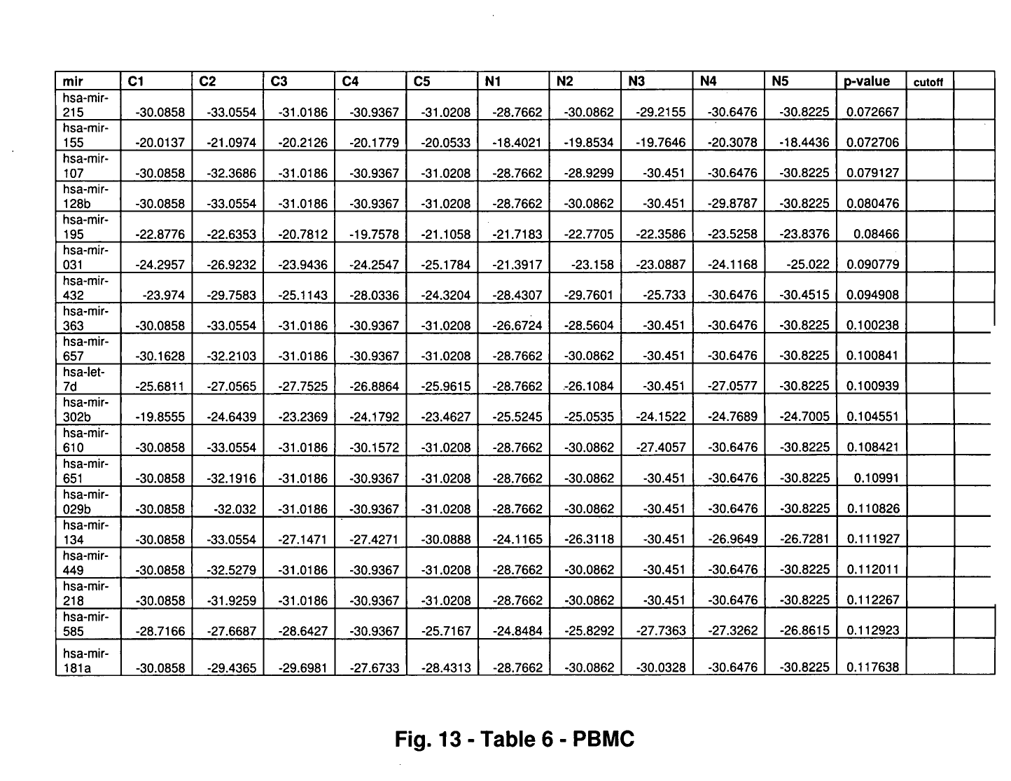

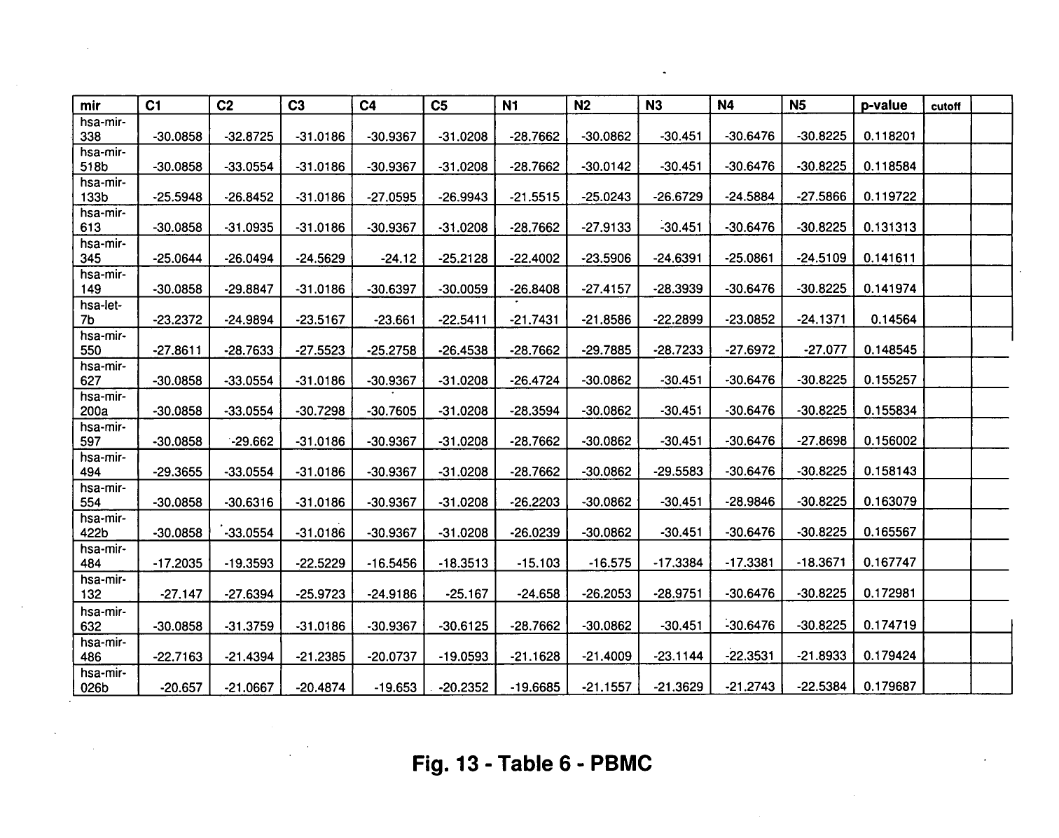

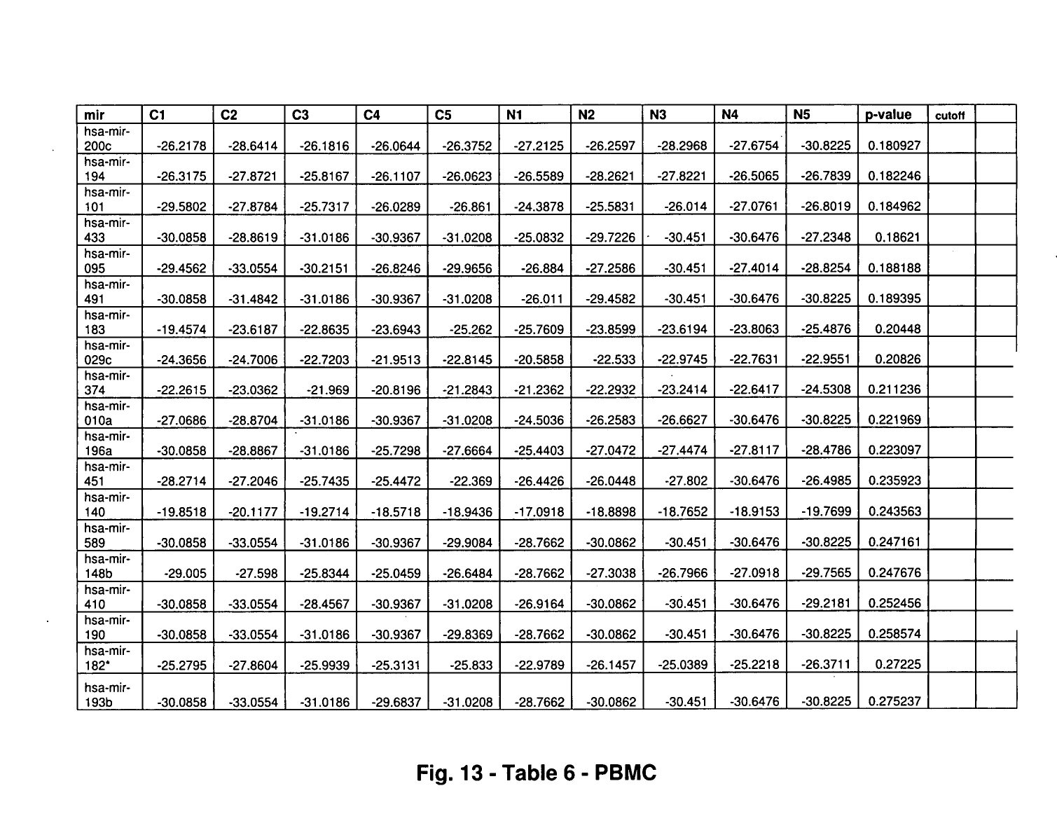

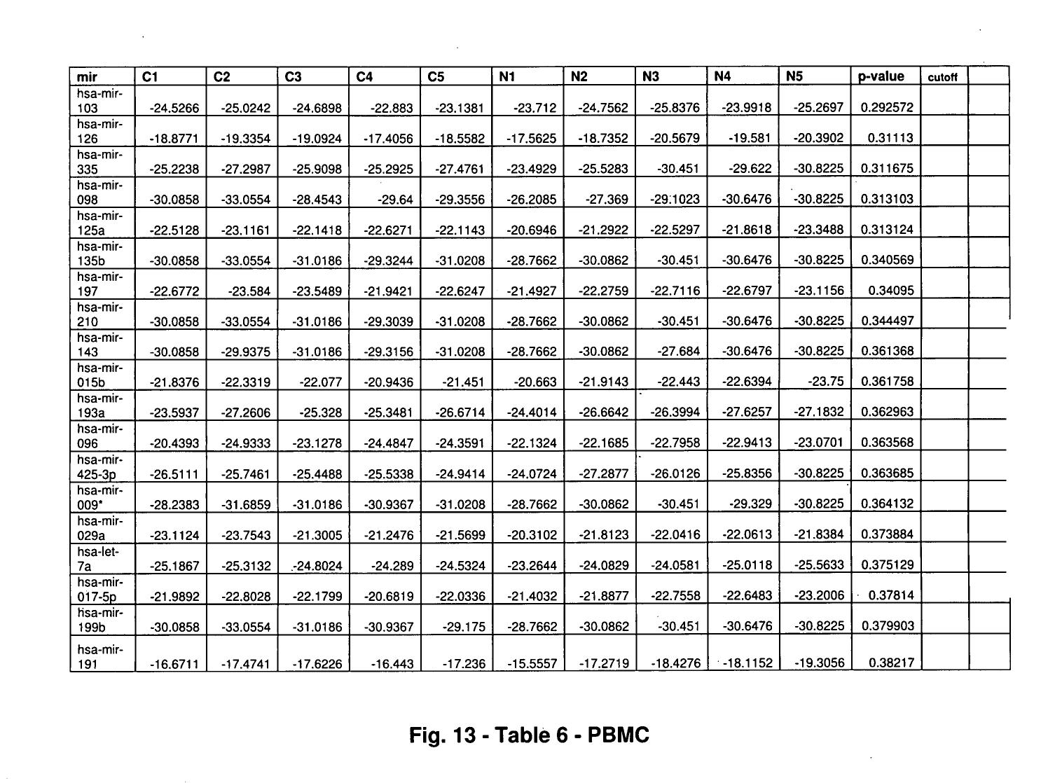

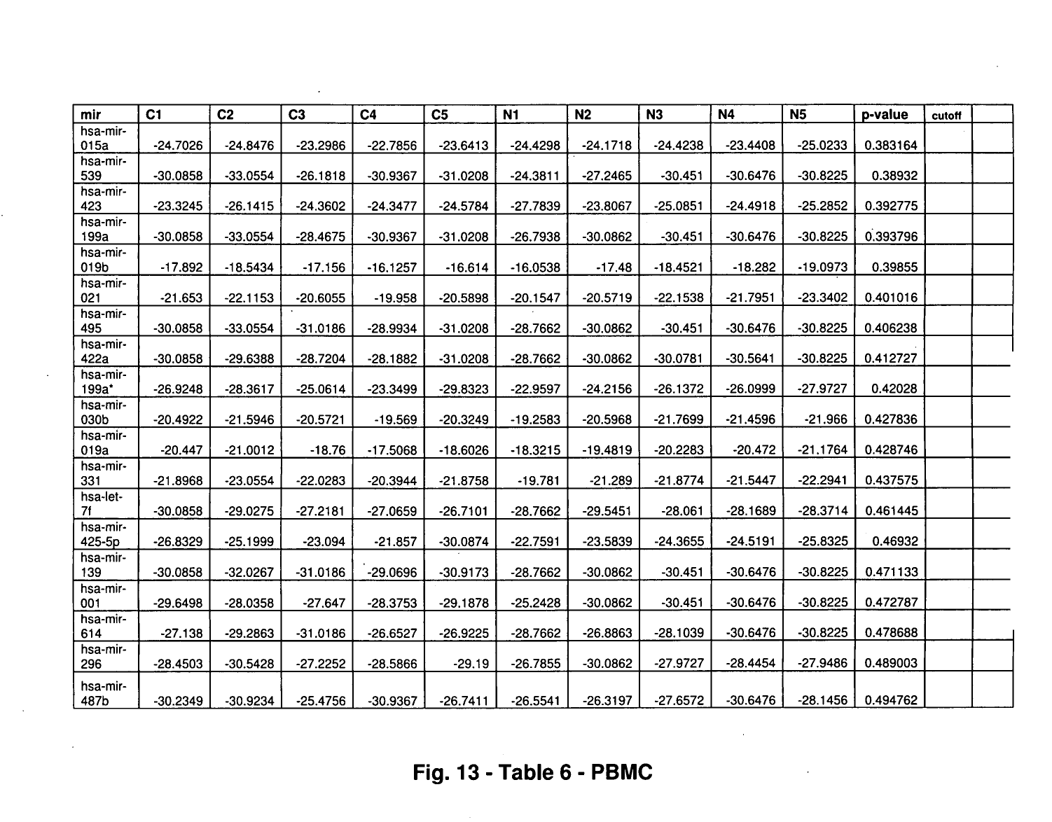

FIG. 11 (Actual figure says FIG. 9 at the bottom)—Table 4 showing the Oligoprobes, the Precursor Sequences, the Mature mRNA, whether the Probe is on the active site, the Entrez-Gene ID, the Ref Seq ID, the miRBase Stem Loop Accession Number, the miRBase Mature Sequence Accession Number, Notes, the Oligo Sequences, the Mature miRNA Sequences, and the Stem Loop Sequences.

FIG. 12—Table 5 showing miRNAs detected in serum.

FIG. 13—Table 6 showing miRNAs detected in peripheral blood mononuclear cells (PBMCs).

### DETAILED DESCRIPTION OF THE PREFERRED EMBODIMENT(S)

The present invention is based, in part, on the identification of specific microRNAs (miRNAs) that are involved in an inflammatory response and/or have altered expression levels in blood. The invention is further based, in part, on association of these miRNAs with particular diagnostic, prognostic and therapeutic features.

In a first broad aspect, there is provided herein a method of determining whether a subject has, or is at risk for developing, one or more lung cancer associated diseases. The method generally includes: measuring the level of at least one miR gene product in a peripheral blood sample from the subject, where an alteration in the level of the miR gene product in the sample, relative to the level of a corresponding miR gene product in a control sample, is indicative of the subject either having, or being at risk for developing, one or more lung cancer associated diseases.

In certain embodiments, the peripheral blood sample comprises one or more of: whole blood, peripheral blood mononuclear cells (PBMC) and serum.

In certain embodiments, the one or more lung cancer associated diseases comprise bronchoalveolar carcinoma (BAC), non-small cell lung cancer (NSCLC), lung adenocarcinoma, lung squamous cell carcinoma and small cell carcinoma.

In certain embodiments, the peripheral blood sample comprises whole blood, and at least one miR gene product is one or more miR gene products selected from the group shown in Table 1 herein having an increased expression relative to a normal control. In certain embodiments, the miRs are one or more of: hsa-miR-518f, hsa-miR-516-3p and hsa-miR-516-5p.

In certain embodiments, the peripheral blood sample comprises whole blood, and wherein at least one miR gene product is one or more miR gene products selected from the group shown in Table 1 herein having a decreased miR expression of relative to a normal control. In certain embodiments, the miR are one or more of: hsa-miR-1-2No1, hsa-miR-511-2No2, hsa-miR-101-2No1, hsa-miR-218-2-precNo1, hsa-miR-451No2, hsa-miR-126*No2, hsa-let-7d-v1-prec, hsa-miR-1-1No1, hsa-miR-123-precNo1, hsa-miR-100No1, hsa-miR-150-prec, hsa-miR-021-prec-17No1, hsa-miR-34aNo1, hsa-let-7iNo1, hsa-miR-126*No1, hsa-miR-126No2, hsa-miR-181c-precNo2, hsa-miR-126No1.

In another aspect, the peripheral blood sample comprises peripheral blood mononuclear cells PBMC), and at least one miR gene product is one or more miR gene products selected from the group shown in Table 2 consisting of an decreased miR expression of: hsa-miR-630.

In another aspect, the sample comprises peripheral blood mononuclear cells, and at least one miR gene product is one or more miR gene products selected from the group shown in Table 2 consisting of a increased miR expression of: hsa-miR-152, hsa-miR-365, hsa-miR-487a, hsa-miR-148a, hsa-miR-636, hsa-miR-320 and hsa-miR-145.

In another aspect, the peripheral blood sample comprises serum, and at least one miR gene product is one or more miR gene products selected from the group shown in Table 3 consisting of an increased miR expression of: hsa-miR-192.

In another aspect, the sample comprises serum, and at least one miR gene product is one or more miR gene products selected from the group shown in Table 3 consisting of a decreased miR expression of: hsa-miR-532, hsa-miR-197, hsa-miR-342.

In another aspect, the at least one miR gene product is one or more miR gene products selected from the group shown in Table 4.

In another aspect, the at least one miR gene product is one or more miR gene products selected from the group shown in Table 5.

In another aspect, the at least one miR gene product is one or more miR gene products selected from the group shown in Table 6.

In another aspect, the method is used for determining the prognosis of a subject with lung cancer, comprising: measuring the level of at least one miR gene product in the sample from the subject, wherein the miR gene product is associated with an adverse prognosis in lung cancer; and, an alteration in the level of the at least one miR gene product in the sample, relative to the level of a corresponding miR gene product in a control sample, is indicative of an adverse prognosis.

In another aspect, there is provided herein a method of detecting one or more lung cancer associated diseases in a peripheral blood sample, the method comprising: analyzing the sample for the altered expression of at least one biomarker associated with lung cancer, and correlating the altered expression of the at least one biomarker with the presence or absence of lung cancer in the sample, where the at least one biomarker is selected from the miRs listed in Table 1, Table 2 or Table 3.

In another aspect, there is provided herein a method of early diagnosing a subject suspected of having one or more lung cancer associated diseases, the method comprising: obtaining a sample from the subject; analyzing the sample for the altered expression of at least one biomarker associated with lung cancer; correlating the altered expression of at least one biomarker with the presence of lung cancer in the subject; where the at least one biomarker is selected from the miRs listed in Table 1, Table 2 or Table 3.

In another aspect, there is provided herein a method of treating a subject with one or more lung cancer associated diseases, comprising administering a therapeutically effective amount of a composition comprising a nucleic acid complementary to at least one of biomarker selected from the group consisting of the miRs listed in Table 1, Table 2 or Table 3.

In another aspect, there is provided herein a pharmaceutical composition comprising a nucleic acid complementary to at least one biomarker selected from the group consisting of the miRs listed in Table 1, Table 2 or Table 3.

In another aspect, there is provided herein a method of comparing peripheral blood samples in a patient having undergone chemoradiation therapy for one or more lung cancer associated diseases and samples of patients not having undergone chemoradiation therapy, comprising: comparing differential expression of at least one of biomarker selected from the group consisting of the miRs listed in Table 1, Table 2 or Table 3.

In another aspect, there is provided herein a method of comparing staging in one or more lung cancer associated diseases in a patient, comprising: obtaining a peripheral blood sample from the patient; and comparing differential expression of at least one of biomarker selected from the group consisting of the miRs listed in Table 1, Table 2 or Table 3.

In another aspect, there is provided herein a method for suppressing one or more lung cancer associated diseases in a subject in need thereof, comprising: administering at least one miRs listed in Table 1, Table 2 or Table 3.

In another aspect, there is provided herein a method of treating one or more lung cancer associated diseases in a subject suffering there from in which at least one miR is down-regulated or up-regulated in the cancer cells of the subject relative to control cells, comprising: when the at least one miR is down-regulated in the cancer cells, administering to the subject an effective amount of at least one isolated miR, such that proliferation of cancer cells in the subject is inhibited; or when the at least one miR is up-regulated in the cancer cells, administering to the subject an effective amount of at least one compound for inhibiting expression of the at least one miR, such that proliferation of cancer cells in the subject is inhibited; wherein the miR is selected from the group consisting of the miRs listed in Table 1, Table 2 or Table 3.

In another aspect, there is provided herein a method of treating one or more lung cancer associated diseases in a subject, comprising: determining the amount of at least one miR in a peripheral blood sample obtained from the subject, relative to a control sample, wherein the miR is selected from the miRs listed in Table 1, Table 2 or Table 3; and altering the amount of miR activity in the subject by: (i) administering to the subject an effective amount of at least one isolated miR, if the amount of the miR expressed in the subject is less than the amount of the miR expressed in control cells; or (ii) administering to the subject an effective amount of at least one compound for inhibiting expression of the at least one miR, if the amount of the miR expressed in the subject is greater than the amount of the miR expressed in control cells, such that proliferation of lung cancer in the subject is inhibited.

In another aspect, there is provided herein a method of identifying an anti-lung cancer related disease agent, comprising: providing a test agent to an cancer cell, and measuring the level of at least one miR associated with decreased expression levels in the lung cancer cell, where an increase in the level of the miR in the lung cancer cell, relative to a suitable control cell, is indicative of the test agent being an anti-cancer agent; wherein the miR is selected from the group consisting of the miRs listed in Table 1, Table 2 or Table 3.

In another aspect, there is provided herein a method for assessing a pathological condition, or the risk of developing a pathological condition, in a subject comprising: measuring an expression profile of one or more markers in a sample from the subject, where a difference in the expression profile in the sample from the subject and an expression profile of a normal sample is indicative of one or more lung cancer associated diseases or a predisposition thereto, and where the marker at least comprises one or more miRs listed in Table 1, Table 2 or Table 3.

In another aspect, there is provided herein a composition comprising one or more of the miR is selected from the group consisting of the miRs listed in Table 1, Table 2 or Table 3.

In another aspect, there is provided herein a reagent for testing for one or more lung cancer associated diseases, wherein the reagent comprises a polynucleotide comprising the nucleotide sequence of at least one miR listed in Table 1, Table 2 or Table 3, or a nucleotide sequence complementary to the nucleotide sequence of the miR.

In another aspect, there is provided herein a reagent for testing for one or more lung cancer associated diseases, wherein the reagent comprises an antibody that recognizes a protein encoded by at least one miR listed in Table 1, Table 2 or Table 3.

In another aspect, there is provided herein a method of assessing the effectiveness of a therapy to prevent, diagnose and/or treat one or more lung cancer associated diseases, comprising: subjecting a subject to a therapy whose effectiveness is being assessed, and determining the level of effectiveness of the treatment being tested in treating or preventing one or more lung cancer associated diseases, by evaluating at least one miR listed in Table 1, Table 2 or Table 3.

In certain embodiments, the candidate therapeutic agent comprises one or more of: pharmaceutical compositions, nutraceutical compositions, and homeopathic compositions.

In certain embodiments, the therapy being assessed is for use in a human subject.

In another aspect, there is provided herein an article of manufacture comprising: at least one capture reagent that binds to a marker for one or more lung cancer associated diseases selected from at least one of the miRs listed in Table 1, Table 2 or Table 3.

In another aspect, there is provided herein a kit for screening for a candidate compound for a therapeutic agent to treat one or more lung cancer associated diseases, wherein the kit comprises: one or more reagents of at least one miR listed in Table 1, Table 2 or Table 3 and a cell expressing at least one miR.

In certain embodiments, the presence of the miR is detected using a reagent comprising an antibody or an antibody fragment which specifically binds with at least one miR.

In another aspect, there is provided herein a screening test for one or more lung cancer associated diseases comprising: contacting one or more of the miRs listed in Table 1, Table 2 or Table 3with a substrate for such miR and with a test agent, and determining whether the test agent modulates the activity of the miR.

In certain embodiments, all method steps are performed in vitro.

In another aspect, there is provided herein use of an agent that interferes with one or more lung cancer associated response signaling pathway, for the manufacture of a medicament for treating, preventing, reversing or limiting the severity of one or more lung cancer associated disease related complications in an individual, wherein the agent comprises at least one miR listed in Table 1, Table 2 or Table 3.

In another aspect, there is provided herein a method of treating, preventing, reversing or limiting the severity of one or more lung cancer associated disease complications in an individual in need thereof, comprising: administering to the individual an agent that interferes with at least one or more lung cancer associated disease response cascade, wherein the agent comprises at least one miR listed in Table 1, Table 2 or Table 3.

In another aspect, there is provided herein use of an agent that interferes with at least one or more lung cancer associated disease response cascade, for the manufacture of a medicament for treating, preventing, reversing or limiting the severity of one or more lung cancer -related disease complication in an individual, wherein the agent comprises at least one miR listed in Table 1, Table 2 or Table 3.

### Examples

The invention may be better understood by reference to the following non-limiting examples, which serve to illustrate but not to limit the present invention.

Accordingly, the invention encompasses methods of diagnosing whether a subject has, or is at risk for developing, lung cancer. According to one aspect, the level of at least one miR gene product in a test sample from the subject is compared to the level of a corresponding miR gene product in a control sample. An alteration (e.g., an increase, a decrease) in the level of the miR gene product in the test sample, relative to the level of a corresponding miR gene product in a control sample, is indicative of the subject either having, or being at risk for developing, lung cancer. In certain embodiments, the test sample comprises peripheral blood.

While not wishing to be bound by theory, it is now believed by the inventors herein that miRNA expression profiles are detectable in the peripheral blood and are useful in assessing lung cancer in a subject. It is also now shown herein that the miRNA expression profiles are useful to distinguish subjects with early stage lung cancer from both those with late stage disease and and further to distinguish current/former smokers without lung cancer.

Also, microRNAs in the peripheral blood are now believed by the inventors herein to reflect primary tumor biology and are now useful in the diagnosis, surveillance of lung cancer disease progression/recurrence and to monitor responses to therapy.

The inventors herein have now identified distinct miRNA expression profiling (i.e., miR signatures or biomarkers) in the peripheral blood of subjects with documented lung cancer.

In a particular aspect, the inventors herein identified the presence of miRNAs in the peripheral blood of both subjects with advanced lung cancer and a set of non-smoker subjects without known lung cancer. Initial unsupervised cluster analysis demonstrates the presence of miRNA that discriminate between the two groups.

MiRNA profiling is a useful tool to identify biologically relevant targets. While the role of miRNA in peripheral blood remains unknown, the inventors herein believe that peripheral blood miRNA profiling is useful to identify distinct molecular signatures in lung cancer and to correlate such profiles with tumor biology. These signatures can be used to complement other modalities, such as, for example, microarray/proteomic platforms and CT scanning; thus supporting a personalized approach to lung cancer diagnosis and treatment.

The inventors also now believe that microRNAs identified in the peripheral blood reflect primary tumor biology and are useful as biomarkers for disease detection, for determining response to therapy, and for surveillance of lung cancers, and/or for monitoring any recurrence of lung cancer. Furthermore the miRNAs are useful to demonstrate distinct networks of molecular pathways, which, in turn are useful in identifying new therapeutic targets.

As shown in the examples herein, there are distinct miRNA signatures that exist in lung tumors from former/current and never smokers. These signatures are useful to identify biological targets and pathways.

MiRNA signatures were identified in smoking and non-smoking individuals with lung cancer and matched controls. The presence of distinct miRNA expression patterns in tumors are to be evaluated in the following groups: 1—Resectable subjects with Non-small cell lung cancer (NSCLC) who are either current or former smokers; 2—Resectable subjects with Non-small cell lung cancer (NSCLC) who are never smokers; and 3—Healthy controls.

The presence of distinct miRNA signatures in both tumors and peripheral blood serves to distinguish current/former smokers and never smokers with lung cancer subjects from controls. Also, the peripheral blood miRNA expression patterns reflect the primary tumor signature.

Lung Cancer Specific miRNAs

The causes of altered expression of miRNAs in cancer are not well understood. However, at least five main mechanisms have recently been identified: 1) miRNA location at cancer-associated genomic regions; 2) Epigenetic regulation; 3) Disruption in miRNA processing proteins and genes such as Dicer and Drosha; 4) miRNA-miRNA interaction; and, 5) Targeting of miRNA expression by oncogenes and tumor suppressor genes.

MicroRNA Biogenesis and Targeting

MiRNAs may be located in several genomic locations, such as within introns of protein coding genes or within introns or exons of noncoding RNAs. Within the nucleus, miRNAs are transcribed as long primary transcripts by RNA polymerase II into primary miRNAs (pri-miRNAs), which range from hundreds to thousands of nucleotides in length.

While in the nucleus, Drosha cleaves both strands of the pri-miRNA to release a 70- to 100-nucleotide stem loop, termed the precursor miRNA (pre-miRNA). The pre-miRNA is subsequently exported from the nucleus to the cytoplasm by the Exportin5/RanGTP. Once in the cytoplasm, a second RNase III (termed Dicer), in conjunction with a dsRBD, cleaves the pre-miRNA, releasing an approximately 22-nucleotide RNA duplex (mature miRNA and its complement miRNA*).

Only one strand of the miRNA/miRNA* duplex is released to enter the protein complex of miRNA-containing ribonucleoprotein particles (miRNPs), and the other strand is degraded. MiRNPs guide miRNAs to the target RNA to regulate protein expression by either translational inhibition or mRNA degradation. MiRNAs bind to target sites in the 3′-untranslated regions of protein coding transcripts. Repression of translation and mRNA degradation are dependent on base-pairing between the “seed” region at the 5′ end of the miRNA and the target site. Most miRNAs have multiple targets and thus the ability to regulate hundreds to thousands of genes.

The inventors herein have examined whole peripheral blood miRNA expression in a cohort of four subjects with advanced NSCLC and three normal controls. Whole peripheral blood from four subjects with documented advanced (stage 3B, IV) non-small cell lung cancer and three healthy control subjects was examined. MiRNA chip analysis in these individuals demonstrated the presence of 93 miRNAs that were either up- or down regulated in the peripheral blood of lung cancer subjects compared to normal (data not shown). A cutoff of two-fold change was used to signify significant miRNAs.

The inventors herein have identified the presence of miRNAs in the peripheral blood of both subjects with advanced lung cancer and a set of non-smokers without known lung cancer.

In addition, initial unsupervised cluster analysis demonstrates the presence of miRNA that discriminate between the two groups of individuals at extremes of disease (smokers with advanced lung cancer and non-smokers without disease). It is to be noted, however, that these results do not take into account changes attributable to smoking history, or co-morbid diseases.

Localizing miRNA and specific targets in human lung cancer tissue is an important step to determining key biological pathways developing in vitro models based relevant cell types.

The inventors have demonstrated in situ hybridization as a method for localizing miRNAs in lung tumors. The inventors observed that mature miR-155 is not present in adenocarcinoma but is present in bronchoalveolar carcinoma (BAC). This finding suggests that differences may exist in both miRNA regulation and is now believe to have biological relevance in these subtypes of lung cancer.

Methods

For clarity, smoking related lung adenocarcinomas are confined to subjects with >20 pack years smoking history. They were either current or former smokers. Only subjects with <100 lifetime cigarettes were included as “never-smokers.” It was believed that most subjects with BAC would be nonsmokers, but patient groups were not restricted by this clinical characteristic. Frequency matching was performed for recruiting subjects for three groups, i.e., age.

The inventors have now have found that, consistent with the national data, approximately 13% of the subjects report never smoking. Also consistent with national data, approximately 40-45% of subjects with lung cancer have adenocarcinoma. With respect to BAC, while a fair number of subjects have BAC features, the tumor registry reports that 76 subjects were diagnosed with mucinous or nonmucinous BAC from 2000-2006.

Samples were quick frozen in liquid nitrogen and stored at (−) 80° C. Current and former smokers without a history or current diagnosis of lung cancer (previous chest radiograph) were studied along with evaluating a group of healthy never smokers. The subjects are appropriately matched for age, sex, and co-morbid illness.

Sample Size and Power Calculation

The inventors compared respectable/unresectable current/former smoker group with control and never smoking groups for differences in microRNA expression levels whole peripheral blood. The inventors separated hypothesis testing into a priori interesting microRNAs (93 microRNA found in preliminary data and 43 microRNAs listed in Yanaihara et al., 2006) versus exploring the whole human microRNAs.

For the 125 a priori interesting microRNAs, the inventors avoided 4 false positives using the generalized familywise error rate approach (GFWER) of Lehman and Romano (2005). A sample size of 80 (20 controls, 40 resectable current/former smoker, and 20 resectable never smokers) allowed the inventors to detect a 2-fold difference in expression with >80% power given the median standard deviation found in the preliminary data.

For the whole genome exploration, there were approximately 180 microRNAs testable on the chip. The inventors used a GFWER of 0.05 and allowed 10 false positives. With 80 samples for three groups, the inventors had 80% power to detect fold difference of 1.9. Background correction, filtering, and normalization methods was performed to avoid technical bias. T-tests were performed to detect differentially expressed microRNAs. In order to improve the estimates of the variability and statistical tests for differential expression, a shrinking variance estimation method was employed. The p-values are assessed by nonparametric approaches (Westfall and Young, 1993).

Blood Processing

Blood samples were obtained from subjects regardless of whether they underwent surgical resection (i.e., subjects who plan to have surgery, radiation, chemotherapy or no treatment are eligible for having blood samples procured). The inventors obtained whole blood samples (5 cc) in two separate PAX-GENE (commercially available) tubes. Samples were then processed through a modified TRizol extraction protocol for whole blood RNA.

For analysis in serum and PBMCs, the inventors have employed a second method for miRNA analysis. As a second method, the inventors were able to obtain sufficient RNA (5-10 μg) from the serum fraction from 18 cc of peripheral blood. Peripheral blood was collected in EDTA-tubes. The blood was diluted 1:2 with sterile PBS then layered over Ficoll-Histopaque (d=1.077) and centrifuged. The resultant plasma was subjected directly to RNA isolation using Trizol. The mononuclear cell layer containing lymphocytes and monocytes were washed once in sterile PBS prior to RNA isolation. Neutrophils were isolated from the red cell pellet by dextran-sulfate sedimentation RBC will be subjected to hyptonic lysis prior to RNA extraction.

MicroRNA Analysis

Whole blood miRNA expression was analyzed by miRNA chip through the OSU-CCC Microarray facility and by RT-PCR for known miRNAs. RNA was isolated from whole blood, PBMC, serum and lung tissue and processed. The microarray facility utilized a microRNACHIP v3 that contains probes against 578 precursor miRNA sequences (329 Homo sapiens, 249 Mus Musculus and 3 Arabidopsis thaliana). 5 μg of total RNA was prepared by generation of first strand cDNA followed by array hybridization to each OSU-CCC miRNA chip. Once completed, miRNA targets were identified utilizing Sanger miRBase 7.0 (Target scan. Pictar).

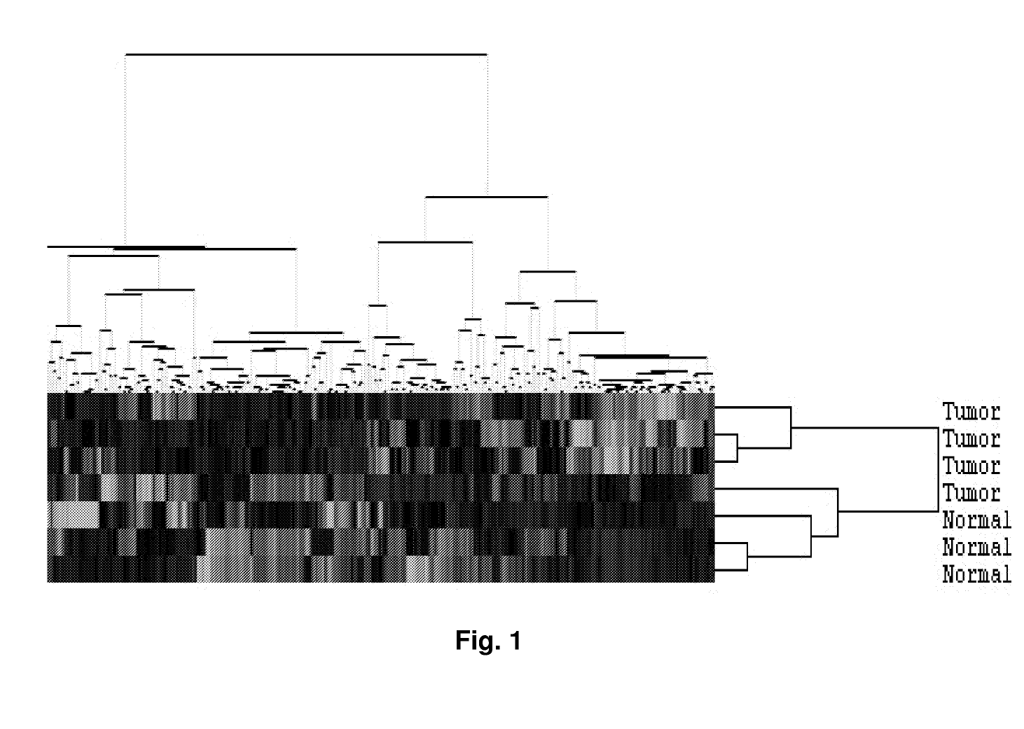

FIG. 1 illustrates miRNA Biogenesis which shows that the miRNA signatures were identified in individuals with lung cancer and matched controls. The miRNAs are detectable in the peripheral blood of individuals with documented lung cancer

Peripheral Blood miRNAs Profiles

Peripheral blood miRNAs profiles are useful to distinguish between individuals with documented lung cancer prior to therapy and individuals without lung cancer.

Whole peripheral blood miRNAs were analyzed using SAM (Significance Analysis of Microarray) software to identify statistically significant miRNAs in the two classes (peripheral blood of subjects with lung cancer: Tumor versus subjects without documented lung cancer or lung disease (Normal).

### Example 1

Peripheral Whole Blood microRNA Expression Correlates with Previously Reported Primary Tumor Expression for Specific microRNAs.

The inventors identified miRNAs that were down-regulated both in lung tumors and in peripheral blood samples from subjects with advanced lung cancer. See Table 1 which shows that miRNAs were altered in the peripheral blood of a group of subjects.

Table 1 showing miRNAs increased and decreased in Lung Cancer relative to Normal levels in Whole Blood. The score (d), the fold change and the q-value (%) are shown.

TABLE 1
Whole Blood - Lung cancer relative to Normal

| | | Fold | |
|---|---|---|---|
| | Score(d) | Change | q-value(%) |
| Increased | | | |
| miR | | | |
| hsa-mir-518f | 3.345799 | 12.52680204 | 8.310609012 |
| hsa-mir-516-3,5p | 2.811169 | 5.026500011 | 15.1371807 |
| hsa-mir-517b* | 1.87141 | 4.216286579 | 39.5584989 |
| hsa-mir-490No2 | 1.66293 | 4.319016049 | 46.2679085 |
| hsa-mir-139-prec | 1.633882 | 5.260833194 | 46.2679085 |
| hsa-mir-007-2-precNo1 | 1.561578 | 4.255803003 | 46.2679085 |
| hsa-mir-021-prec-17No2 | 1.530267 | 5.483822338 | 46.2679085 |
| hsa-mir-106bNo2 | 1.472306 | 2.970512063 | 46.2679085 |
| hsa-mir-345No2 | 1.354315 | 3.070818501 | 46.2679085 |
| hsa-mir-217-precNo1 | 1.330406 | 2.88509924 | 48.93559237 |
| hsa-mir-323No2 | 1.249622 | 3.715040183 | 48.93559237 |
| hsa-mir-218-2-precNo2 | 1.245357 | 2.043647257 | 48.93559237 |
| hsa-mir-202 | 1.222944 | 4.169921717 | 48.93559237 |
| hsa-mir-425No1 | 1.219679 | 2.259308936 | 48.93559237 |
| hsa-mir-096-prec-7No1 | 1.212552 | 2.274999124 | 48.93559237 |
| hsa-mir-125a-precNo2 | 1.192433 | 2.051344216 | 48.93559237 |
| hsa-mir-339No1 | 1.169387 | 2.533722194 | 48.93559237 |
| hsa-mir-141-precNo1 | 1.168474 | 1.448661387 | 48.93559237 |
| hsa-mir-321No1 | 1.141995 | 3.327155819 | 51.11780052 |
| Decreased | | | |
| miR | | | |
| hsa-mir-1-2No1 | −5.14606 | 0.047824083 | 0 |
| hsa-mir-511-2No2 | −2.88051 | 0.165332748 | 0 |
| hsa-mir-101-2No1 | −2.32058 | 0.202612615 | 3.53200883 |
| hsa-mir-218-2-precNo1 | −2.243 | 0.190737123 | 3.53200883 |
| hsa-mir-451No2 | −2.16462 | 0.128361863 | 5.468916898 |
| hsa-miR-126*No2 | −2.10064 | 0.171875137 | 5.468916898 |
| hsa-let-7d-v1-prec | −1.98573 | 0.393308775 | 8.310609012 |
| hsa-mir-1-1No1 | −1.93063 | 0.263696268 | 8.310609012 |
| hsa-mir-123-precNo1 | −1.92838 | 0.286070753 | 8.310609012 |
| hsa-mir-100No1 | −1.88085 | 0.259908534 | 8.310609012 |
| hsa-mir-150-prec | −1.71005 | 0.272634755 | 10.76421739 |
| hsa-mir-021-prec-17No1 | −1.69611 | 0.360401527 | 10.76421739 |
| hsa-mir-34aNo1 | −1.68418 | 0.165059864 | 10.76421739 |
| hsa-let-7iNo1 | −1.65132 | 0.316399997 | 10.76421739 |
| hsa-mir-017-precNo2 | −1.57194 | 0.360232851 | 13.83970807 |
| hsa-mir-001b-2-prec | −1.57008 | 0.302842475 | 13.83970807 |
| hsa-miR-126*No1 | −1.55629 | 0.336928919 | 13.83970807 |
| hsa-mir-20bNo1 | −1.54706 | 0.34676152 | 13.83970807 |
| hsa-mir-202-prec | −1.53388 | 0.355708941 | 13.83970807 |
| hsa-mir-020-prec | −1.53116 | 0.341576672 | 13.83970807 |
| hsa-mir-383No1 | −1.5047 | 0.467216864 | 13.83970807 |
| hsa-let-7d-v2-precNo2 | −1.49932 | 0.381044968 | 13.83970807 |
| hsa-let-7g-precNo1 | −1.47253 | 0.344891902 | 15.1371807 |
| hsa-mir-106aNo1 | −1.46639 | 0.387284633 | 15.1371807 |
| hsa-mir-126No2 | −1.43898 | 0.334968526 | 15.1371807 |
| hsa-mir-018-prec | −1.43559 | 0.39771395 | 16.80749029 |
| hsa-mir-206-precNo1 | −1.4322 | 0.308245813 | 16.80749029 |
| hsa-mir-009-1No1 | −1.41482 | 0.25831765 | 16.80749029 |
| hsa-mir-181c-precNo2 | −1.37505 | 0.357463144 | 17.30684327 |
| hsa-let-7b-prec | −1.35902 | 0.431540157 | 17.30684327 |
| hsa-mir-007-3-precNo1 | −1.32461 | 0.29034566 | 18.62331929 |
| hsa-mir-103-2-prec | −1.29216 | 0.475306554 | 20.09320579 |
| hsa-mir-219-2No2 | −1.249 | 0.245194988 | 23.28141032 |
| hsa-mir-016a-chr13 | −1.23992 | 0.444044882 | 23.28141032 |
| hsa-mir-126No1 | −1.23377 | 0.240395843 | 23.28141032 |
| hsa-mir-106-prec-X | −1.22828 | 0.399041588 | 23.28141032 |
| hsa-mir-107No1 | −1.21849 | 0.47853172 | 24.86534216 |
| hsa-mir-196-1-precNo1 | −1.20669 | 0.416869233 | 24.86534216 |
| hsa-mir-106bNo1 | −1.17879 | 0.43289157 | 24.86534216 |
| hsa-let-7f-1-precNo2 | −1.16055 | 0.413064069 | 27.90506355 |
| hsa-mir-107-prec-10 | −1.153 | 0.486350342 | 27.90506355 |
| hsa-let-7a-1-prec | −1.12818 | 0.495703067 | 30.66614725 |
| hsa-mir-144-precNo2 | −1.11119 | 0.396174535 | 30.66614725 |
| hsa-let-7d-prec | −1.05984 | 0.512636421 | 32.37027873 |
| hsa-mir-320No2 | −1.03127 | 0.543595227 | 35.28190442 |
| hsa-mir-21No1 | −1.02203 | 0.527911281 | 35.28190442 |
| hsa-mir-103-prec-5 = 103-1 | −1.00614 | 0.53554009 | 35.28190442 |
| hsa-mir-516-2No1 | −0.99436 | 0.28029219 | 35.28190442 |
| hsa-mir-001b-1-prec1 | −0.98761 | 0.543890931 | 39.5584989 |
| hsa-mir-125b-2-precNo2 | −0.96728 | 0.464152331 | 39.5584989 |
| hsa-mir-130a-precNo2 | −0.96441 | 0.495594292 | 39.5584989 |
| hsa-mir-030b-precNo2 | −0.96377 | 0.612299695 | 39.5584989 |
| hsa-let-7a-2-precNo2 | −0.95718 | 0.538253165 | 39.5584989 |
| hsa-mir-132-precNo2 | −0.94949 | 0.510690813 | 39.5584989 |
| hsa-mir-516-45p | −0.9178 | 0.569620457 | 41.2281758 |
| hsa-mir-374No1 | −0.91109 | 0.599855388 | 41.2281758 |
| hsa-mir-015a-2-precNo1 | −0.8707 | 0.569973854 | 46.2679085 |
| hsa-mir-517a | −0.86295 | 0.614382807 | 48.93559237 |
| hsa-mir-016b-chr3 | −0.85717 | 0.564255595 | 48.93559237 |
| hsa-mir-017-precNo1 | −0.84955 | 0.631686009 | 48.93559237 |
| hsa-mir-148-prec | −0.80917 | 0.612327748 | 51.11780052 |

### Example 2

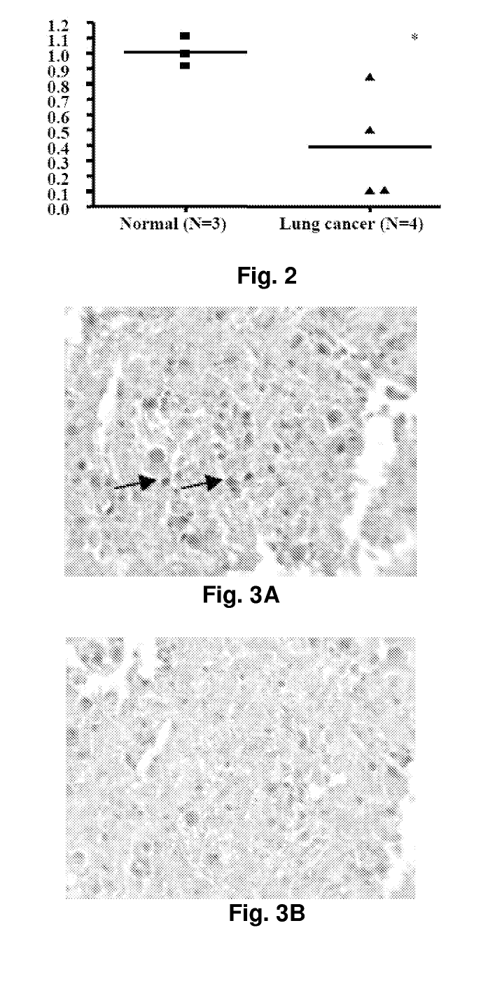

In addition, the inventors confirmed whole peripheral blood expression patterns of a specific miRNA (miR-126) by RT-PCR (see FIG. 2) and in cases of non small cell lung cancer (NSCLC) (n=4) compared to normal controls (n=3).

### Example 3

In situ Hybridization is Useful to Identify miRNA Location in Mammalian Tissues.

MiRNA expression profiling of lung tissue distinguishes lung cancers from normal lung tissue. In situ hybridization studies were used to locate potential miRNAs in human lung cancer tissue samples. In one non-limiting example, miR-155 is increased in expression in several solid and hematological malignancies. In lung cancer, increased miR-155 expression correlates with poor survival.

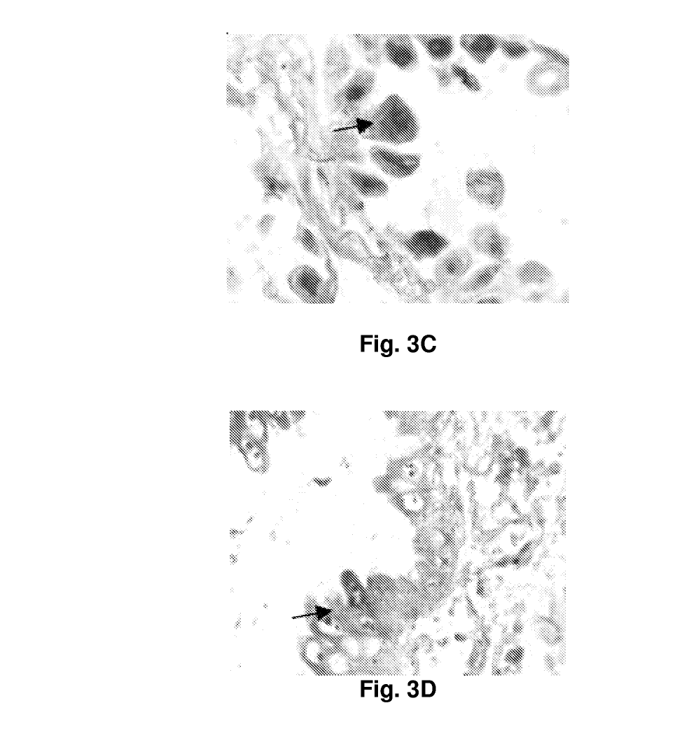

However, both the location and regulation of miR-155 expression in lung cancer remain unknown. As now shown herein, the premature form of miR-155 has been identified in the nucleus of cancerous cells in adenocarcinoma but the mature form was not easily identified. (See FIGS. 3A, 3B).

While not wishing to be bound by theory, the inventors herein now believe that this shows impaired processing of miR-155 as a potential mechanism for regulation in NSCLC. In bronchoalveolar cell carcinoma, both premature and mature forms of miR-155 were present at high levels. (See FIG. 3C, 3D) This finding represents an example of the heterogeneity that exists in microRNA expression in subtypes of lung cancer.

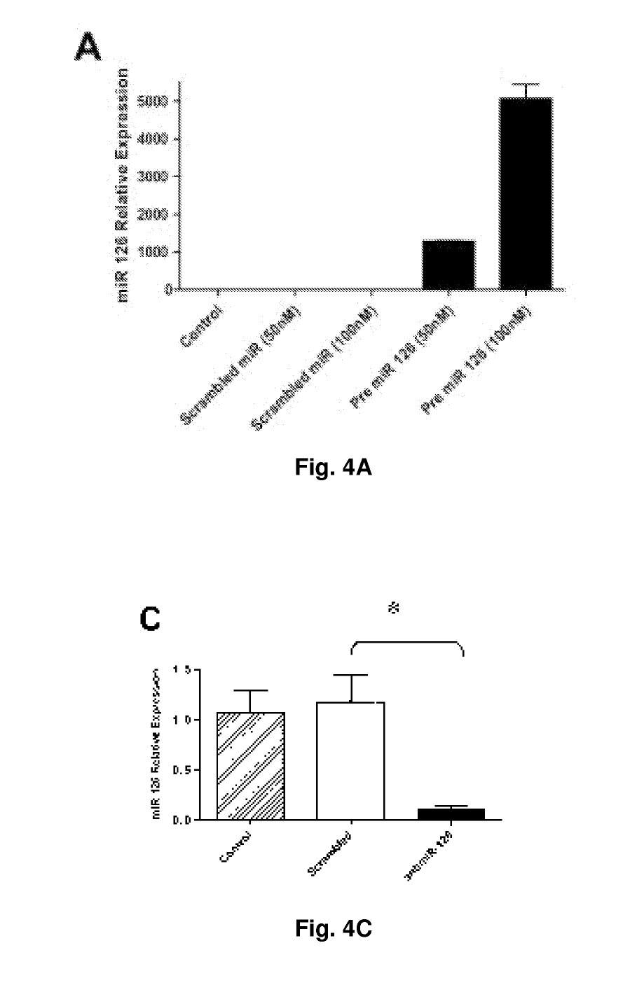

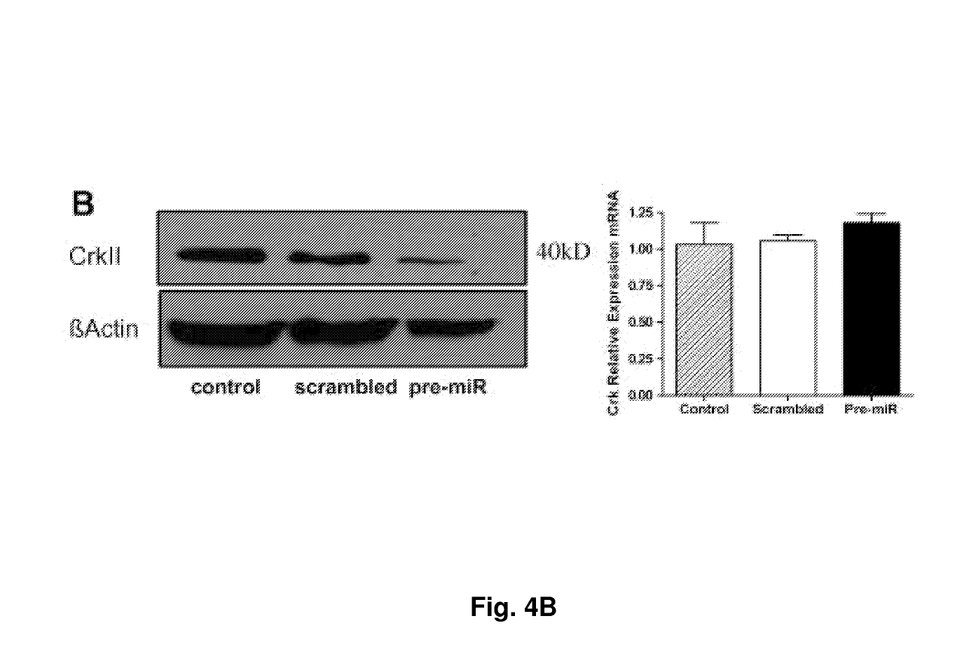

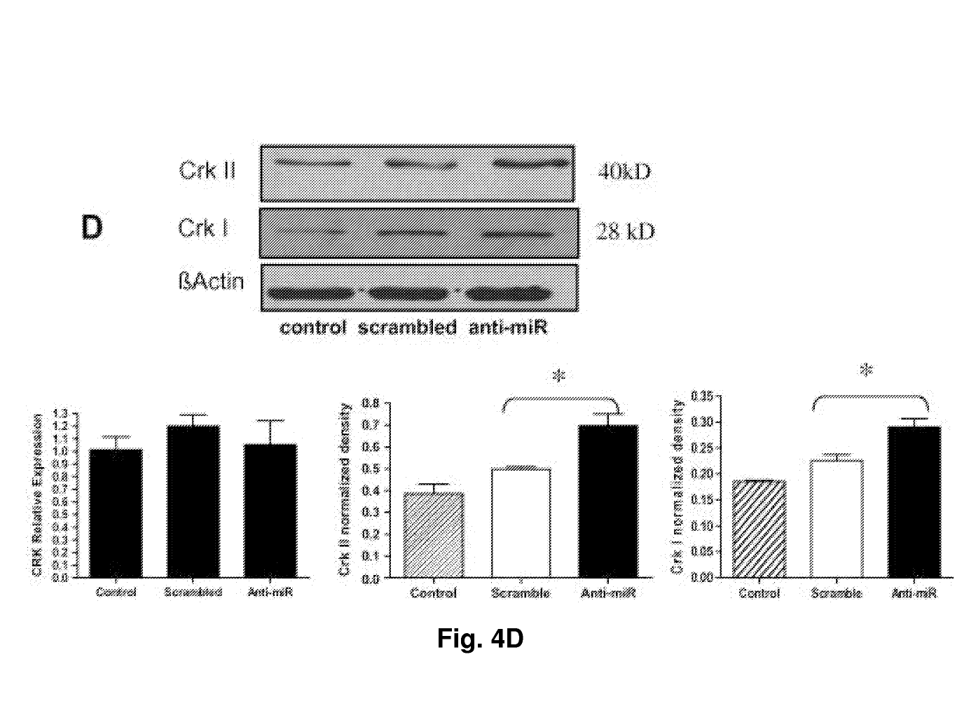

FIGS. 4A-4D show that MiR-126 transfection alters Crk protein expression. Crk is an adaptor protein implicated in several malignancies including lung cancer and predicted target for miR-126. FIG. 4A shows premiR-126 transfection of H1703 cells resulted in a 1000- to 5000-fold increase in miR-126 mRNA expression and a decrease in Crk II protein.

FIGS. 4B-4D show that, with no change in Crk mRNA (FIG. 4B), Crk I protein was not detectable by Western. Transfection of H226 cells (squamous cell) with 100 nM of LNA miR-126 anti-sense oligonucleotide resulted in a 10-fold decrease in miR-126 expression compared to scrambled pre-miR transfection (FIG. 4C) and increase in Crk II protein expression as measured by densitometry (*p<0.05) but no change in mRNA (D). Western blots were conducted in duplicate and all RT-PCR results represent average=/−S.E. from two independent experiments conducted in duplicate. (*p<0.05 scrambled versus pre-miR) 18S was used as an internal control.

### Example 4

Targeted MiRNA Silencing is Useful to Examine Resultant Alterations in Cell Phenotype.

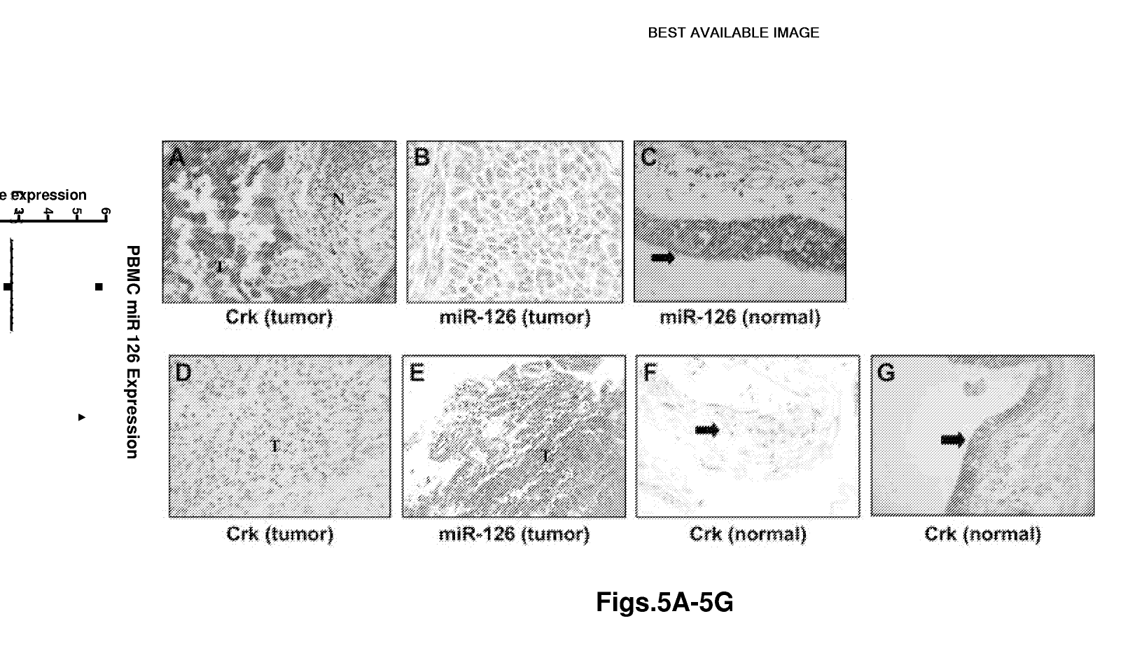

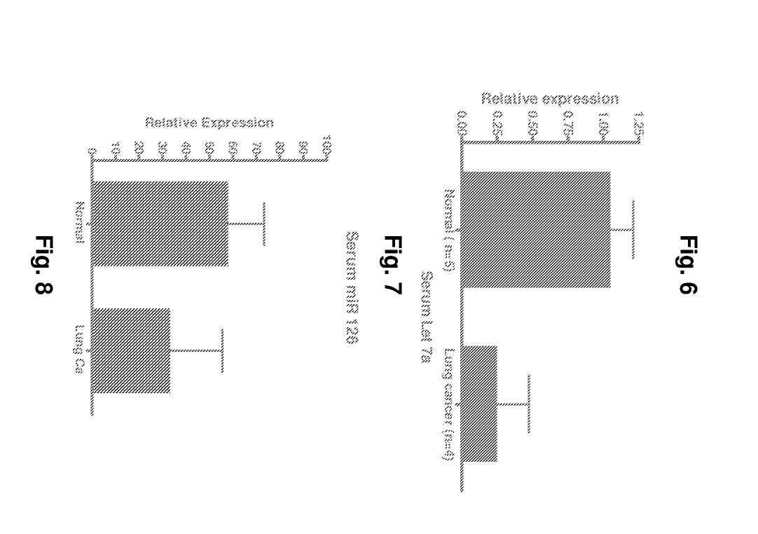

FIGS. 5A-5G show representative images demonstrating in situ hybridization for miR-126 and immunohistochemistry for Crk in human squamous cell carcinomas of the lung. In case one, Crk expression (red) is evident within most tumor cells (FIG. 5A) while there is a lack of miR-126 expression in the adjacent section in the tumor cells (FIG. 5B) whereas miR-126 was detected in normal bronchial epithelium (FIG. 5C) (blue signal). In case number two, there is no detectable Crk within the tumor (FIG. 5D) while there is strong expression of miR-126 (blue) within the tumor (FIG. 5E); Crk localized to the endothelium (FIG. 5F); and to the bronchial epithelium (FIG. 5G) in normal tissue (All images are at 400× with the exception of FIG. 5F which is 1000× and FIG. 5G which is 200×).

MiR-126 has multiple predicted targets including CRK a signaling adaptor protein that has been shown to activate kinase signaling and anchorage-independent growth in vitro.

### Example 5

Screening Tests for Early Detection of Lung Cancer

Until the present invention, there have been no established screening tests for early detection of lung cancer, and less than 25% of subjects present with surgically curable disease (stages I and II). In addition, disease recurrence remains unacceptably high. Recent epidemiologic statistics reveal that the majority of lung cancers are now diagnosed in former and never smokers. There is little known about the distinct genetic and epigenetic events that lead to the development of either bronchoalveolar carcinoma (BAC) of non-BAC adenocarcinoma in subjects who have never smoked.

Lung Cancer Specific miRNAs

The strategies determining miRNA function for tissues/cells and disease models include: a) Determining the effects of in vitro targeted over-expression and silencing of select lung cancer specific microRNAs on disease phenotype, and b) identifying regulation and processing of select lung cancer specific microRNAs.

### Example 6

Table 2 show miRNAs that are increased and decreased in Lung Cancer relative to Normal levels in Peripheral Blood Mononuclear Cells (PBMC). Table 2 show the data presented as delta CT (internal control 18s minus sample) concerning the miRNAs found in Peripheral Blood Mononuclear Cells (PBMC) for cancer (C) and normal (N) for C1, C2, C3, C5, N1, N2, N3, N4, and N5.

TABLE 2
Peripheral Blood Mononuclear Cells (PBMC)

| Decreased | | | | | | |
|---|---|---|---|---|---|---|
| miR | Cancer 1 | Cancer 2 | Cancer 3 | Cancer 4 | Cancer 5 | |
| hsa-miR- | −30.031181 | −31.577905 | −31.018573 | −30.385778 | −31.020805 | |
| 630 | | | | | | |
| | Normal 1 | Normal 2 | Normal 3 | Normal 4 | Normal 5 | p-value |
| hsa-miR- | −27.628025 | −26.359519 | −29.363501 | −30.314921 | −29.423825 | 0.03396097 |
| 630 | | | | | | |
| Increased | | | | | | |
| miR | Cancer 1 | Cancer 2 | Cancer 3 | Cancer 4 | Cancer 5 | |
| hsa-miR- | −24.696657 | −26.724839 | −24.795845 | −23.177116 | −25.591518 | |
| 152 | | | | | | |
| hsa-miR- | −29.498964 | −28.526082 | −28.370375 | −28.003646 | −27.763481 | |
| 365 | | | | | | |
| hsa-miR- | −24.852018 | −27.000349 | −25.631475 | −24.732046 | −25.507969 | |
| 487a | | | | | | |
| hsa-miR- | −26.640607 | −26.617635 | −27.651455 | −24.724729 | −25.889485 | |
| 148a | | | | | | |
| hsa-miR- | −21.744566 | −27.791383 | −27.263 | −26.890791 | −26.921815 | |
| 636 | | | | | | |
| hsa-miR- | −22.020717 | −23.913751 | −21.909175 | −21.365478 | −21.905893 | |
| 320 | | | | | | |
| hsa-miR- | −27.524816 | −28.207952 | −28.841915 | −26.701532 | −27.019565 | |
| 145 | | | | | | |
| | Normal 1 | Normal 2 | Normal 3 | Normal 4 | Normal 5 | p-value |
| hsa-miR- | −26.197838 | −30.086169 | −30.450953 | −29.295783 | −30.822515 | 0.003365049 |
| 152 | | | | | | |
| hsa-miR- | −28.766243 | −30.086169 | −30.450953 | −30.647601 | −30.822515 | 0.007132 |
| 365 | | | | | | |
| hsa-miR- | −25.982845 | −28.239104 | −27.854433 | −27.712634 | −26.797967 | 0.015057463 |
| 487a | | | | | | |
| hsa-miR- | −26.102363 | −30.086169 | −30.450953 | −30.135466 | −30.260915 | 0.016142469 |
| 148a | | | | | | |
| hsa-miR- | −28.766243 | −30.086169 | −30.450953 | −30.647601 | −30.822515 | 0.018827827 |
| 636 | | | | | | |
| hsa-miR- | −22.265163 | −24.507059 | −23.900527 | −24.164501 | −25.120412 | 0.026020896 |
| 320 | | | | | | |
| hsa-miR- | −28.766243 | −27.737839 | −30.450953 | −30.647601 | −30.822515 | 0.027402602 |
| 145 | | | | | | |

### Example 7

Table 3 show miRNAs that are increased and decreased in Lung Cancer relative to Normal levels in Serum. These are presented as delta CT (internal control 18S minus sample). Table 3 shows the data concerning the miRNAs found in serum for cancer (C) and normal (N) for C1, C2, C3, C5, N1, N2, N3, N4, and N5.

TABLE 3
Serum

| Increased | | | | | | |
|---|---|---|---|---|---|---|
| miR | Cancer 1 | Cancer 2 | Cancer 3 | Cancer 5 | | |
| hsa-miR- | −21.736865 | −15.902032 | −18.122697 | −15.227855 | | |
| 192 | | | | | | |
| | Normal 1 | Normal 2 | Normal 3 | Normal 4 | Normal 5 | p-value |
| hsa-miR- | −26.148583 | −24.109533 | −23.537545 | −27.195413 | −24.489854 | 0.008900968 |
| 192 | | | | | | |
| Decreased | | | | | | |
| miR | Cancer 1 | Cancer 2 | Cancer 3 | Cancer 5 | | |
| hsa-miR- | −29.08123 | −25.58978 | −23.35198 | −26.511243 | | |
| 532 | | | | | | |
| hsa-miR- | −22.13607 | −20.030474 | −18.809703 | −18.117963 | | |
| 197 | | | | | | |
| hsa-miR- | −21.300728 | −20.484504 | −21.466453 | −16.887311 | | |
| 342 | | | | | | |
| | Normal 1 | Normal 2 | Normal 3 | Normal 4 | Normal 5 | p-value |
| hsa-miR- | −22.024838 | −21.034307 | −21.544165 | −22.518178 | −24.005956 | 0.037548656 |
| 532 | | | | | | |
| hsa-miR- | −16.323512 | −17.326522 | −16.580279 | −17.310384 | −17.084718 | 0.044654737 |
| 197 | | | | | | |
| hsa-miR- | −15.195003 | −16.77618 | −16.218703 | −18.06808 | −17.49974 | 0.046073537 |
| 342 | | | | | | |

### Example 8

FIG. 6 is a graph showing relative expression of miR-126 in lung cancer relative to normal levels in Peripheral Blood Mononuclear Cells (PBMC).

Also, see FIG. 7 which contains a graph showing relative expression of miR-let 7a in lung cancer relative to normal levels in Serum. FIG. 8 contains a graph showing relative expression of miR-126 in lung cancer relative to normal levels in Serum.

### Example 9

FIGS. 9A-9D: Effects of miR-126 over-expression on H1703 proliferation, adhesion, migration and invasion.

FIG. 9A: Control, scrambled pre-miR and pre-miR 126 cells exhibited similar rates of growth over 96 h. Two independent proliferation assays were conducted in triplicate.

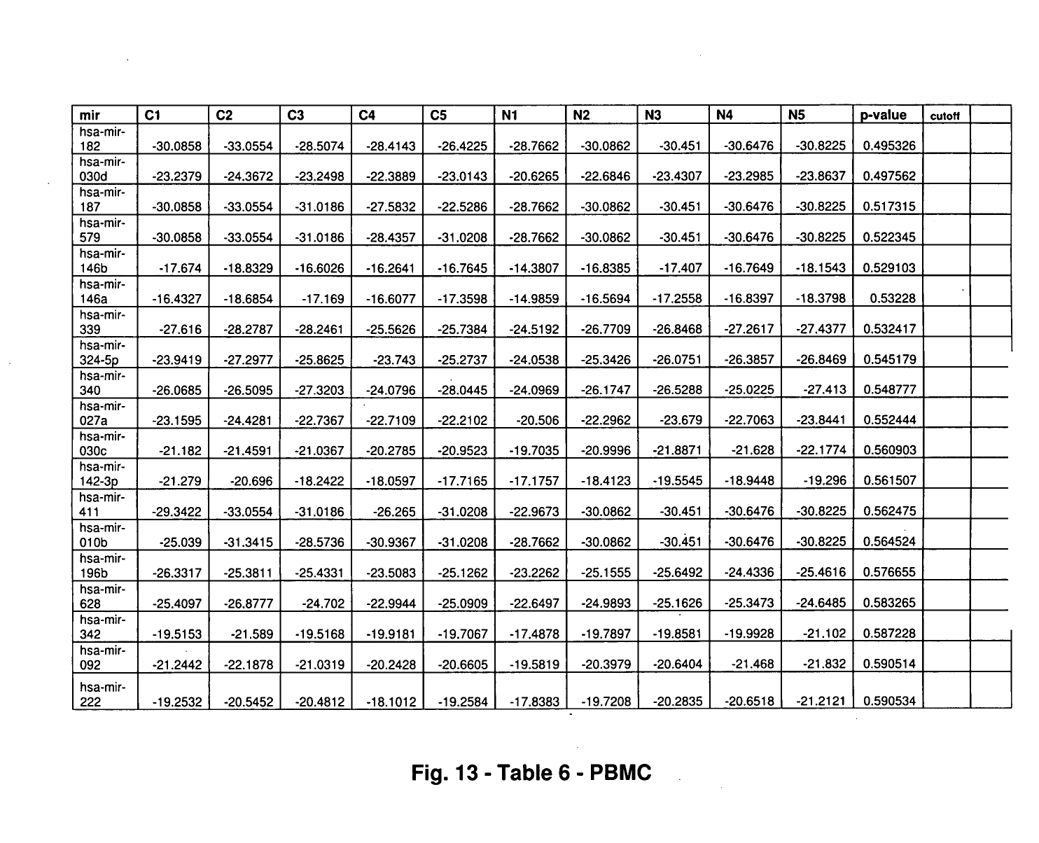

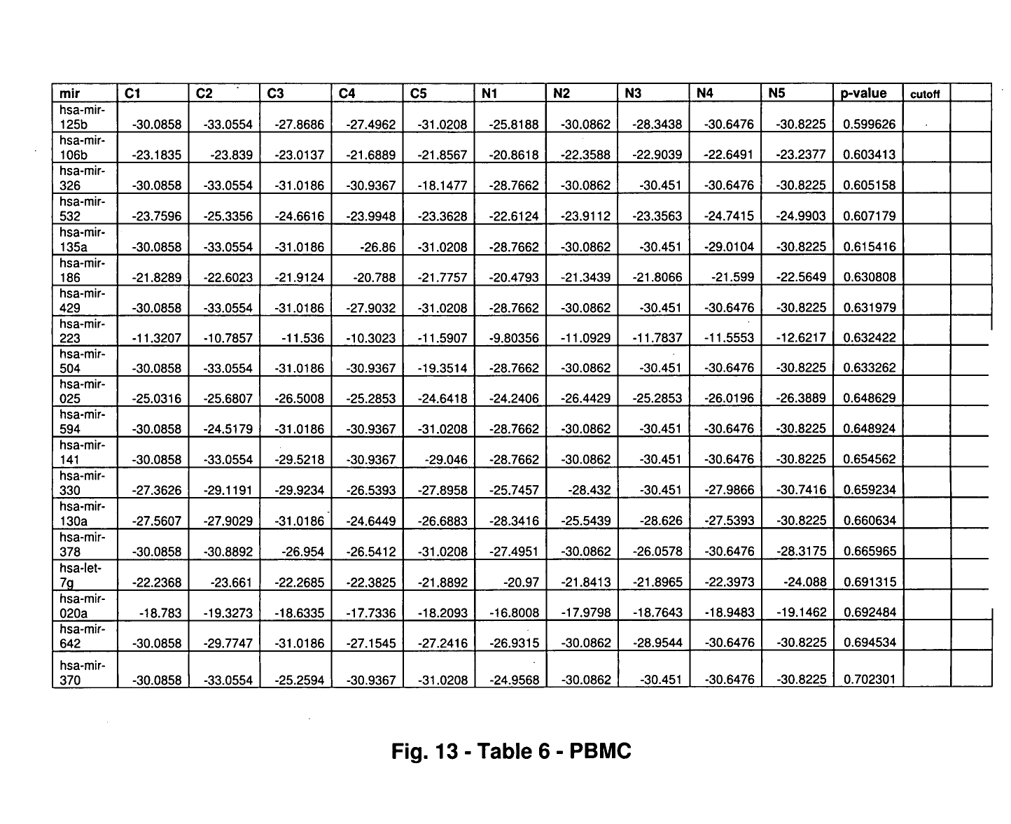

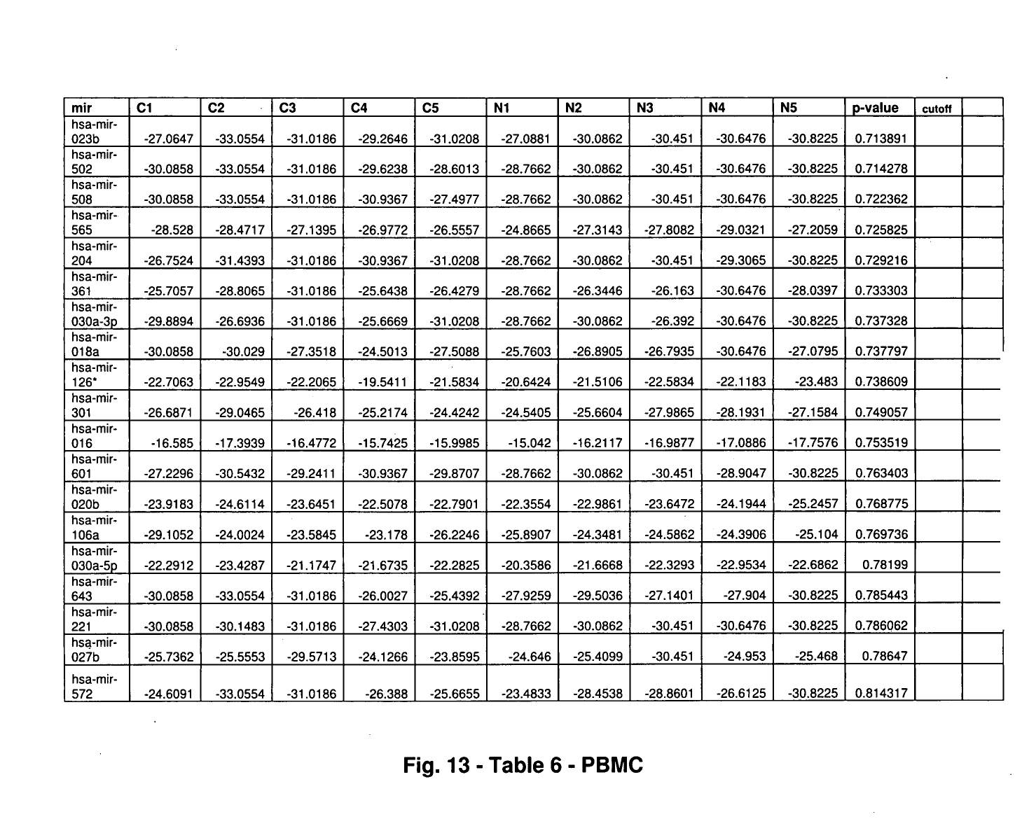

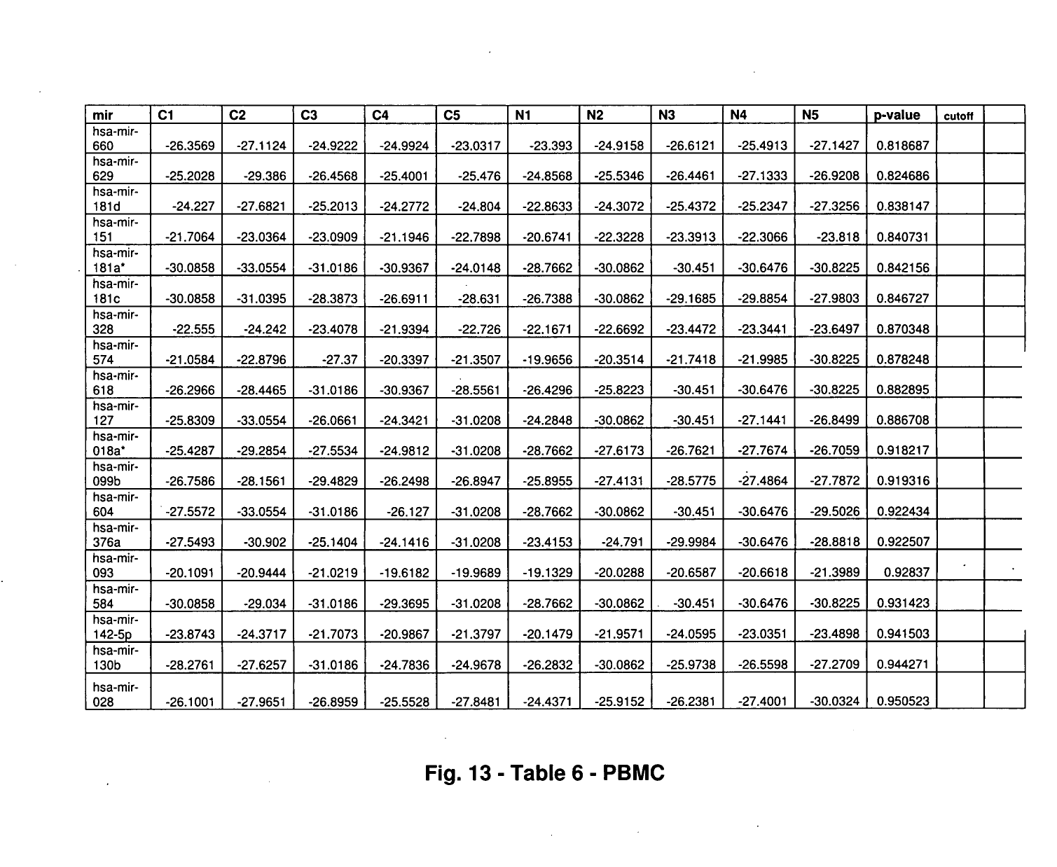

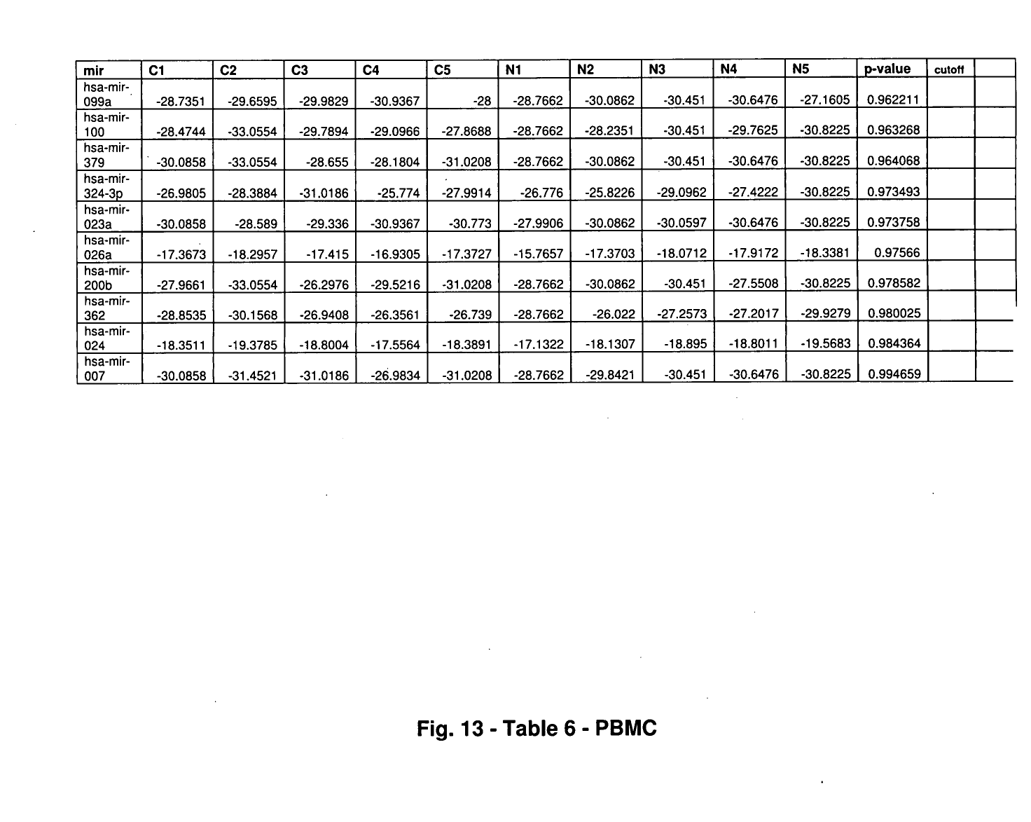

FIGS. 92B-92D: MiR-126 over-expressing cells demonstrated decreased adherence (FIG. 9B), migration (FIG. 9C) and invasion (FIG. 9D). Images in FIG. 9C and FIG. 9D are representative of blinded random fields (p<0.05). In all experiments, miR-126 over-expression was confirmed by RT-PCR to ensure adequate induction. Results represent average of four fields conducted in triplicate (*p<0.05 scrambled versus pre-miR).

### Example 10

FIGS. 10A-10B: miR-126 and Crk expression in NSCLC tissues: Examination of 19 pairs of human non-small cell lung cancers and uninvolved adjacent lung (squamous 1-13 and adenocarcinoma 14-19) demonstrate a decrease in miR-126 mRNA expression in tumors (T) compared to uninvolved adjacent normal (N) lung (FIG. 10A). Crk mRNA expression in these same samples was variable with seven out of 19 tumors exhibiting higher Crk expression than uninvolved adjacent lung. (FIG. 10B) RT-PCR results represent average=/−S.E. from two independent experiments conducted in duplicate (*p<0.05 tumor versus uninvolved lung). 18S was used as the endogenous control

In situ Hybridization for Localization of Select Premature and Nature miRNAs

MiRNAs implicated in tumorigenesis are now believed by the inventors herein to differ in regulation and biological relevance depending on lung cancer cell type. The inventors now believe that there is a distinct signature of miRNA expression in lung tumors from current/former smokers and never smokers. Furthermore, the inventors have identified a group of miRNAs relevant to lung tumorigenesis.

### Example 11

miRNA Analysis Using Real-Time PCR.

The expression of 500 mature human miRNAs can be profiled by real-time PCR to discover miRNAs that are differentially expressed in the blood from patients with lung cancer and normal controls. RNA (50 ng) can be converted to cDNA by priming with a mixture of looped primers to 500 known human mature miRNAs (Mega Plex kit, Applied Biosystems) using previously published reverse transcription conditions. Primers to the internal controls snoRNAs U38B and U43 as well as 18S and 7S rRNA can be included in the mix of primers. The expression can be profiled using an Applied Biosystems 7900HT real-time PCR instrument equipped with a 384 well reaction plate.

Liquid-handling robots and the Zymak Twister robot can be used to increase throughput and reduce error. Real-time PCR can be performed using standard conditions. The optimal internal control can be determined by comparing the mean 2^{-CT} of the GOLD classes. This internal control can be used to calculate the relative gene expression. Relative expression of each miRNA can be calculated from the equation 2^{-ΔCT}, where ΔCT=CTmiRNA−CTinternal control.

The PCR based relative miRNA expression can then be analyzed using t tests. The −ΔCT data can be analyzed using the method of hierarchical clustering and the results plotted in a heatmap. Additional statistical analysis such as ANOVA can be performed to determine miRNAs that are differentially expressed between lung cancer and normal levels.

FIG. 11—Table 4 shows a listing of the Oligoprobes, the Precursor Sequences, the Mature mRNA, whether the Probe is on the active site, the Entrez-Gene ID, the Ref Seq ID, the miRBase Stem Loop Accession Number, the miRBase Mature Sequence Accession Number, Notes, the Oligo Sequences, the Mature miRNA Sequences, and the Stem Loop Sequences.

### Example 12

FIG. 12—Table 5 shows miRNAs detected in serum.

### Example 13

FIG. 13—Table 6 shows miRNAs detected in peripheral blood mononuclear cells (PBMCs).

Definitions and Examples of Uses

As used herein interchangeably, a “miR gene product,” “microRNA,” “miR,” “miR” or “miRNA” refers to the unprocessed or processed RNA transcript from a miR gene. As the miR gene products are not translated into protein, the term “miR gene products” does not include proteins. The unprocessed miR gene transcript is also called a “miR precursor,” and typically comprises an RNA transcript of about 70-100 nucleotides in length. The miR precursor can be processed by digestion with an RNAse (for example, Dicer, Argonaut, RNAse III (e.g., E. coli RNAse III)) into an active 19-25 nucleotide RNA molecule. This active 19-25 nucleotide RNA molecule is also called the “processed” miR gene transcript or “mature” miRNA.

The active 19-25 nucleotide RNA molecule can be obtained from the miR precursor through natural processing routes (e.g., using intact cells or cell lysates) or by synthetic processing routes (e.g., using isolated processing enzymes, such as isolated Dicer, Argonaut, or RNAse III). It is understood that the active 19-25 nucleotide RNA molecule can also be produced directly by biological or chemical synthesis, without having to be processed from the miR precursor. When a microRNA is referred to herein by name, the name corresponds to both the precursor and mature forms, unless otherwise indicated.

The methods comprise determining the level of at least one miR gene product in a sample from the subject and comparing the level of the miR gene product in the sample to a control. As used herein, a “subject” can be any mammal that has, or is suspected of having, such disorder. In a preferred embodiment, the subject is a human who has, or is suspected of having, such disorder.

The level of at least one miR gene product can be measured in cells of a biological sample obtained from the subject.

In another embodiment, a sample can be removed from the subject, and DNA can be extracted and isolated by standard techniques. For example, in certain embodiments, the sample can be obtained from the subject prior to initiation of radiotherapy, chemotherapy or other therapeutic treatment. A corresponding control sample, or a control reference sample (e.g., obtained from a population of control samples), can be obtained from unaffected samples of the subject, from a normal human individual or population of normal individuals, or from cultured cells corresponding to the majority of cells in the subject's sample. The control sample can then be processed along with the sample from the subject, so that the levels of miR gene product produced from a given miR gene in cells from the subject's sample can be compared to the corresponding miR gene product levels from cells of the control sample. Alternatively, a reference sample can be obtained and processed separately (e.g., at a different time) from the test sample and the level of a miR gene product produced from a given miR gene in cells from the test sample can be compared to the corresponding miR gene product level from the reference sample.

In one embodiment, the level of the at least one miR gene product in the test sample is greater than the level of the corresponding miR gene product in the control sample (i.e., expression of the miR gene product is “upregulated”). As used herein, expression of a miR gene product is “upregulated” when the amount of miR gene product in a sample from a subject is greater than the amount of the same gene product in a control (for example, a reference standard, a control cell sample, a control tissue sample).

In another embodiment, the level of the at least one miR gene product in the test sample is less than the level of the corresponding miR gene product in the control sample (i.e., expression of the miR gene product is “downregulated”). As used herein, expression of a miR gene is “downregulated” when the amount of miR gene product produced from that gene in a sample from a subject is less than the amount produced from the same gene in a control sample. The relative miR gene expression in the control and normal samples can be determined with respect to one or more RNA expression standards. The standards can comprise, for example, a zero miR gene expression level, the miR gene expression level in a standard cell line, the miR gene expression level in unaffected samples of the subject, or the average level of miR gene expression previously obtained for a population of normal human controls (e.g., a control reference standard).

The level of the at least one miR gene product can be measured using a variety of techniques that are well known to those of skill in the art (e.g., quantitative or semi-quantitative RT-PCR, Northern blot analysis, solution hybridization detection). In a particular embodiment, the level of at least one miR gene product is measured by reverse transcribing RNA from a test sample obtained from the subject to provide a set of target oligodeoxynucleotides, hybridizing the target oligodeoxynucleotides to one or more miRNA-specific probe oligonucleotides (e.g., a microarray that comprises miRNA-specific probe oligonucleotides) to provide a hybridization profile for the test sample, and comparing the test sample hybridization profile to a hybridization profile generated from a control sample. An alteration in the signal of at least one miRNA in the test sample relative to the control sample is indicative of the subject either having, or being at risk for a particular disorder.

Also, a microarray can be prepared from gene-specific oligonucleotide probes generated from known miRNA sequences. The array may contain two different oligonucleotide probes for each miRNA, one containing the active, mature sequence and the other being specific for the precursor of the miRNA. The array may also contain controls, such as one or more mouse sequences differing from human orthologs by only a few bases, which can serve as controls for hybridization stringency conditions. tRNAs and other RNAs (e.g., rRNAs, mRNAs) from both species may also be printed on the microchip, providing an internal, relatively stable, positive control for specific hybridization. One or more appropriate controls for non-specific hybridization may also be included on the microchip. For this purpose, sequences are selected based upon the absence of any homology with any known miRNAs.

The microarray may be fabricated using techniques known in the art. For example, probe oligonucleotides of an appropriate length, e.g., 40 nucleotides, are 5′-amine modified at position C6 and printed using commercially available microarray systems, e.g., the GeneMachine OmniGrid™ 100 Microarrayer and Amersham CodeLink™ activated slides. Labeled cDNA oligomer corresponding to the target RNAs is prepared by reverse transcribing the target RNA with labeled primer. Following first strand synthesis, the RNA/DNA hybrids are denatured to degrade the RNA templates. The labeled target cDNAs thus prepared are then hybridized to the microarray chip under hybridizing conditions, e.g., 6×SSPE/30% formamide at 25° C. for 18 hours, followed by washing in 0.75×TNT at 37° C. for 40 minutes. At positions on the array where the immobilized probe DNA recognizes a complementary target cDNA in the sample, hybridization occurs. The labeled target cDNA marks the exact position on the array where binding occurs, allowing automatic detection and quantification. The output consists of a list of hybridization events, indicating the relative abundance of specific cDNA sequences, and therefore the relative abundance of the corresponding complementary miRs, in the patient sample. According to one embodiment, the labeled cDNA oligomer is a biotin-labeled cDNA, prepared from a biotin-labeled primer. The microarray is then processed by direct detection of the biotin-containing transcripts using, e.g., Streptavidin-Alexa647 conjugate, and scanned utilizing conventional scanning methods. Image intensities of each spot on the array are proportional to the abundance of the corresponding miR in the patient sample.

The use of the array has several advantages for miRNA expression detection. First, the global expression of several hundred genes can be identified in the same sample at one time point. Second, through careful design of the oligonucleotide probes, expression of both mature and precursor molecules can be identified. Third, in comparison with Northern blot analysis, the chip requires a small amount of RNA, and provides reproducible results using 2.5 μg of total RNA. The relatively limited number of miRNAs (a few hundred per species) allows the construction of a common microarray for several species, with distinct oligonucleotide probes for each. Such a tool allows for analysis of trans-species expression for each known miR under various conditions.

In addition to use for quantitative expression level assays of specific miRs, a microchip containing miRNA-specific probe oligonucleotides corresponding to a substantial portion of the miRNome, preferably the entire miRNome, may be employed to carry out miR gene expression profiling, for analysis of miR expression patterns. Distinct miR signatures can be associated with established disease markers, or directly with a disease state.

According to the expression profiling methods described herein, total RNA from a sample from a subject suspected of having a particular disorder is quantitatively reverse transcribed to provide a set of labeled target oligodeoxynucleotides complementary to the RNA in the sample. The target oligodeoxynucleotides are then hybridized to a microarray comprising miRNA-specific probe oligonucleotides to provide a hybridization profile for the sample. The result is a hybridization profile for the sample representing the expression pattern of miRNA in the sample. The hybridization profile comprises the signal from the binding of the target oligodeoxynucleotides from the sample to the miRNA-specific probe oligonucleotides in the microarray. The profile may be recorded as the presence or absence of binding (signal vs. zero signal). More preferably, the profile recorded includes the intensity of the signal from each hybridization. The profile is compared to the hybridization profile generated from a normal control sample or reference sample. An alteration in the signal is indicative of the presence of, or propensity to develop, the particular disorder in the subject.

Other techniques for measuring miR gene expression are also within the skill in the art, and include various techniques for measuring rates of RNA transcription and degradation.

The invention also provides methods of diagnosing whether a subject has, or is at risk for developing, a particular disorder with an adverse prognosis. In this method, the level of at least one miR gene product, which is associated with an adverse prognosis in a particular disorder, is measured by reverse transcribing RNA from a test sample obtained from the subject to provide a set of target oligodeoxynucleotides. The target oligodeoxynucleotides are then hybridized to one or more miRNA-specific probe oligonucleotides (e.g., a microarray that comprises miRNA-specific probe oligonucleotides) to provide a hybridization profile for the test sample, and the test sample hybridization profile is compared to a hybridization profile generated from a control sample. An alteration in the signal of at least one miRNA in the test sample relative to the control sample is indicative of the subject either having, or being at risk for developing, a particular disorder with an adverse prognosis.

In some instances, it may be desirable to simultaneously determine the expression level of a plurality of different miR gene products in a sample. In other instances, it may be desirable to determine the expression level of the transcripts of all known miR genes correlated with a particular disorder. Assessing specific expression levels for hundreds of miR genes or gene products is time consuming and requires a large amount of total RNA (e.g., at least 20 μg for each Northern blot) and autoradiographic techniques that require radioactive isotopes.

To overcome these limitations, an oligolibrary, in microchip format (i.e., a microarray), may be constructed containing a set of oligonucleotide (e.g., oligodeoxynucleotide) probes that are specific for a set of miR genes. Using such a microarray, the expression level of multiple microRNAs in a biological sample can be determined by reverse transcribing the RNAs to generate a set of target oligodeoxynucleotides, and hybridizing them to probe the oligonucleotides on the microarray to generate a hybridization, or expression, profile. The hybridization profile of the test sample can then be compared to that of a control sample to determine which microRNAs have an altered expression level. As used herein, “probe oligonucleotide” or “probe oligodeoxynucleotide” refers to an oligonucleotide that is capable of hybridizing to a target oligonucleotide. “Target oligonucleotide” or “target oligodeoxynucleotide” refers to a molecule to be detected (e.g., via hybridization). By “miR-specific probe oligonucleotide” or “probe oligonucleotide specific for a miR” is meant a probe oligonucleotide that has a sequence selected to hybridize to a specific miR gene product, or to a reverse transcript of the specific miR gene product.

An “expression profile” or “hybridization profile” of a particular sample is essentially a fingerprint of the state of the sample; while two states may have any particular gene similarly expressed, the evaluation of a number of genes simultaneously allows the generation of a gene expression profile that is unique to the state of the cell. That is, normal samples may be distinguished from corresponding disorder-exhibiting samples. Within such disorder-exhibiting samples, different prognosis states (for example, good or poor long term survival prospects) may be determined. By comparing expression profiles of disorder-exhibiting samples in different states, information regarding which genes are important (including both upregulation and downregulation of genes) in each of these states is obtained.

The identification of sequences that are differentially expressed in disorder-exhibiting samples, as well as differential expression resulting in different prognostic outcomes, allows the use of this information in a number of ways. For example, a particular treatment regime may be evaluated (e.g., to determine whether a chemotherapeutic drug acts to improve the long-term prognosis in a particular subject). Similarly, diagnosis may be done or confirmed by comparing samples from a subject with known expression profiles. Furthermore, these gene expression profiles (or individual genes) allow screening of drug candidates that suppress the particular disorder expression profile or convert a poor prognosis profile to a better prognosis profile.

Alterations in the level of one or more miR gene products in cells can result in the deregulation of one or more intended targets for these miRs, which can lead to a particular disorder. Therefore, altering the level of the miR gene product (e.g., by decreasing the level of a miR that is upregulated in disorder-exhibiting cells, by increasing the level of a miR that is downregulated in disorder-exhibiting cells) may successfully treat the disorder.

Accordingly, the present invention encompasses methods of treating a disorder in a subject, wherein at least one miR gene product is deregulated (e.g., downregulated, upregulated) in the cells of the subject. In one embodiment, the level of at least one miR gene product in a test sample is greater than the level of the corresponding miR gene product in a control or reference sample. In another embodiment, the level of at least one miR gene product in a test sample is less than the level of the corresponding miR gene product in a control sample. When the at least one isolated miR gene product is downregulated in the test sample, the method comprises administering an effective amount of the at least one isolated miR gene product, or an isolated variant or biologically-active fragment thereof, such that proliferation of the disorder-exhibiting cells in the subject is inhibited.

For example, when a miR gene product is downregulated in a cancer cell in a subject, administering an effective amount of an isolated miR gene product to the subject can inhibit proliferation of the cancer cell. The isolated miR gene product that is administered to the subject can be identical to an endogenous wild-type miR gene product that is downregulated in the cancer cell or it can be a variant or biologically-active fragment thereof.

As defined herein, a “variant” of a miR gene product refers to a miRNA that has less than 100% identity to a corresponding wild-type miR gene product and possesses one or more biological activities of the corresponding wild-type miR gene product. Examples of such biological activities include, but are not limited to, inhibition of expression of a target RNA molecule (e.g., inhibiting translation of a target RNA molecule, modulating the stability of a target RNA molecule, inhibiting processing of a target RNA molecule) and inhibition of a cellular process associated with cancer and/or a myeloproliferative disorder (e.g., cell differentiation, cell growth, cell death). These variants include species variants and variants that are the consequence of one or more mutations (e.g., a substitution, a deletion, an insertion) in a miR gene. In certain embodiments, the variant is at least about 70%, 75%, 80%, 85%, 90%, 95%, 98%, or 99% identical to a corresponding wild-type miR gene product.

As defined herein, a “biologically-active fragment” of a miR gene product refers to an RNA fragment of a miR gene product that possesses one or more biological activities of a corresponding wild-type miR gene product. As described above, examples of such biological activities include, but are not limited to, inhibition of expression of a target RNA molecule and inhibition of a cellular process associated with cancer and/or a myeloproliferative disorder. In certain embodiments, the biologically-active fragment is at least about 5, 7, 10, 12, 15, or 17 nucleotides in length. In a particular embodiment, an isolated miR gene product can be administered to a subject in combination with one or more additional anti-cancer treatments. Suitable anti-cancer treatments include, but are not limited to, chemotherapy, radiation therapy and combinations thereof (e.g., chemoradiation).

When the at least one isolated miR gene product is upregulated in the cancer cells, the method comprises administering to the subject an effective amount of a compound that inhibits expression of the at least one miR gene product, such that proliferation of the disorder-exhibiting cells is inhibited. Such compounds are referred to herein as miR gene expression-inhibition compounds. Examples of suitable miR gene expression-inhibition compounds include, but are not limited to, those described herein (e.g., double-stranded RNA, antisense nucleic acids and enzymatic RNA molecules).

In a particular embodiment, a miR gene expression-inhibiting compound can be administered to a subject in combination with one or more additional anti-cancer treatments. Suitable anti-cancer treatments include, but are not limited to, chemotherapy, radiation therapy and combinations thereof (e.g., chemoradiation).

As described herein, when the at least one isolated miR gene product is upregulated in cancer cells, the method comprises administering to the subject an effective amount of at least one compound for inhibiting expression of the at least one miR gene product, such that proliferation of cancer cells is inhibited.

The terms “treat”, “treating” and “treatment”, as used herein, refer to ameliorating symptoms associated with a disease or condition, for example, cancer and/or other condition or disorder, including preventing or delaying the onset of the disease symptoms, and/or lessening the severity or frequency of symptoms of the disease, disorder or condition. The terms “subject”, “patient” and “individual” are defined herein to include animals, such as mammals, including, but not limited to, primates, cows, sheep, goats, horses, dogs, cats, rabbits, guinea pigs, rats, mice or other bovine, ovine, equine, canine, feline, rodent, or murine species. In a preferred embodiment, the animal is a human.

As used herein, an “isolated” miR gene product is one that is synthesized, or altered or removed from the natural state through human intervention. For example, a synthetic miR gene product, or a miR gene product partially or completely separated from the coexisting materials of its natural state, is considered to be “isolated.” An isolated miR gene product can exist in a substantially-purified form, or can exist in a cell into which the miR gene product has been delivered. Thus, a miR gene product that is deliberately delivered to, or expressed in, a cell is considered an “isolated” miR gene product. A miR gene product produced inside a cell from a miR precursor molecule is also considered to be an “isolated” molecule. According to the invention, the isolated miR gene products described herein can be used for the manufacture of a medicament for treating a subject (e.g., a human).

Isolated miR gene products can be obtained using a number of standard techniques. For example, the miR gene products can be chemically synthesized or recombinantly produced using methods known in the art. In one embodiment, miR gene products are chemically synthesized using appropriately protected ribonucleoside phosphoramidites and a conventional DNA/RNA synthesizer. Commercial suppliers of synthetic RNA molecules or synthesis reagents include, e.g., Proligo (Hamburg, Germany), Dharmacon Research (Lafayette, Colo., U.S.A.), Pierce Chemical (part of Perbio Science, Rockford, Ill., U.S.A.), Glen Research (Sterling, Va., U.S.A.), ChemGenes (Ashland, Mass., U.S.A.) and Cruachem (Glasgow, UK).

Alternatively, the miR gene products can be expressed from recombinant circular or linear DNA plasmids using any suitable promoter. Suitable promoters for expressing RNA from a plasmid include, e.g., the U6 or H1 RNA pol III promoter sequences, or the cytomegalovirus promoters. Selection of other suitable promoters is within the skill in the art. The recombinant plasmids of the invention can also comprise inducible or regulatable promoters for expression of the miR gene products in cells (e.g., cancerous cells, cells exhibiting a myeloproliferative disorder).

The miR gene products that are expressed from recombinant plasmids can be isolated from cultured cell expression systems by standard techniques. The miR gene products that are expressed from recombinant plasmids can also be delivered to, and expressed directly in, cells.

The miR gene products can be expressed from a separate recombinant plasmid, or they can be expressed from the same recombinant plasmid. In one embodiment, the miR gene products are expressed as RNA precursor molecules from a single plasmid, and the precursor molecules are processed into the functional miR gene product by a suitable processing system, including, but not limited to, processing systems extant within a cancer cell.

Selection of plasmids suitable for expressing the miR gene products, methods for inserting nucleic acid sequences into the plasmid to express the gene products, and methods of delivering the recombinant plasmid to the cells of interest are within the skill in the art. See, for example, Zeng et al. (2002), Molecular Cell 9:1327-1333; Tuschl (2002), Nat. Biotechnol, 20:446-448; Brummelkamp et al. (2002), Science 296:550-553; Miyagishi et al. (2002), Nat. Biotechnol. 20:497-500; Paddison et al. (2002), Genes Dev. 16:948-958; Lee et al. (2002), Nat. Biotechnol. 20:500-505; and Paul et al. (2002), Nat. Biotechnol. 20:505-508, the entire disclosures of which are incorporated herein by reference. For example, in certain embodiments, a plasmid expressing the miR gene products can comprise a sequence encoding a miR precursor RNA under the control of the CMV intermediate-early promoter. As used herein, “under the control” of a promoter means that the nucleic acid sequences encoding the miR gene product are located 3′ of the promoter, so that the promoter can initiate transcription of the miR gene product coding sequences.

The miR gene products can also be expressed from recombinant viral vectors. It is contemplated that the miR gene products can be expressed from two separate recombinant viral vectors, or from the same viral vector. The RNA expressed from the recombinant viral vectors can either be isolated from cultured cell expression systems by standard techniques, or can be expressed directly in cells (e.g., cancerous cells, cells exhibiting a myeloproliferative disorder).

In other embodiments of the treatment methods of the invention, an effective amount of at least one compound that inhibits miR expression can be administered to the subject. As used herein, “inhibiting miR expression” means that the production of the precursor and/or active, mature form of miR gene product after treatment is less than the amount produced prior to treatment. One skilled in the art can readily determine whether miR expression has been inhibited in cells using, for example, the techniques for determining miR transcript level discussed herein. Inhibition can occur at the level of gene expression (i.e., by inhibiting transcription of a miR gene encoding the miR gene product) or at the level of processing (e.g., by inhibiting processing of a miR precursor into a mature, active miR).

As used herein, an “effective amount” of a compound that inhibits miR expression is an amount sufficient to inhibit proliferation of cells in a subject suffering from cancer and/or a myeloproliferative disorder. One skilled in the art can readily determine an effective amount of a miR expression-inhibiting compound to be administered to a given subject, by taking into account factors, such as the size and weight of the subject; the extent of disease penetration; the age, health and sex of the subject; the route of administration; and whether the administration is regional or systemic.

One skilled in the art can also readily determine an appropriate dosage regimen for administering a compound that inhibits miR expression to a given subject, as described herein. Suitable compounds for inhibiting miR gene expression include double-stranded RNA (such as short- or small-interfering RNA or “siRNA”), antisense nucleic acids, and enzymatic RNA molecules, such as ribozymes. Each of these compounds can be targeted to a given miR gene product and interfere with the expression (e.g., by inhibiting translation, by inducing cleavage and/or degradation) of the target miR gene product.

For example, expression of a given miR gene can be inhibited by inducing RNA interference of the miR gene with an isolated double-stranded RNA (“dsRNA”) molecule which has at least 90%, for example, at least 95%, at least 98%, at least 99%, or 100%, sequence homology with at least a portion of the miR gene product. In a particular embodiment, the dsRNA molecule is a “short or small interfering RNA” or “siRNA.”

Administration of at least one miR gene product, or at least one compound for inhibiting miR expression, will inhibit the proliferation of cells (e.g., cancerous cells, cells exhibiting a myeloproliferative disorder) in a subject who has a cancer and/or a myeloproliferative disorder. As used herein, to “inhibit the proliferation of cancerous cells or cells exhibiting a myeloproliferative disorder” means to kill the cells, or permanently or temporarily arrest or slow the growth of the cells. Inhibition of cell proliferation can be inferred if the number of such cells in the subject remains constant or decreases after administration of the miR gene products or miR gene expression-inhibiting compounds. An inhibition of proliferation of cancerous cells or cells exhibiting a myeloproliferative disorder can also be inferred if the absolute number of such cells increases, but the rate of tumor growth decreases.

A miR gene product or miR gene expression-inhibiting compound can also be administered to a subject by any suitable enteral or parenteral administration route. Suitable enteral administration routes for the present methods include, e.g., oral, rectal, or intranasal delivery. Suitable parenteral administration routes include, e.g., intravascular administration (e.g., intravenous bolus injection, intravenous infusion, intra-arterial bolus injection, intra-arterial infusion and catheter instillation into the vasculature); peri- and intra-tissue injection (e.g., peri-tumoral and intra-tumoral injection, intra-retinal injection, or subretinal injection); subcutaneous injection or deposition, including subcutaneous infusion (such as by osmotic pumps); direct application to the tissue of interest, for example by a catheter or other placement device (e.g., a retinal pellet or a suppository or an implant comprising a porous, non-porous, or gelatinous material); and inhalation. Particularly suitable administration routes are injection, infusion and direct injection into the tumor.

The miR gene products or miR gene expression-inhibition compounds can be formulated as pharmaceutical compositions, sometimes called “medicaments,” prior to administering them to a subject, according to techniques known in the art. Accordingly, the invention encompasses pharmaceutical compositions for treating cancer and/or a myeloproliferative disorder.

The present pharmaceutical compositions comprise at least one miR gene product or miR gene expression-inhibition compound (or at least one nucleic acid comprising a sequence encoding the miR gene product or miR gene expression-inhibition compound) (e.g., 0.1 to 90% by weight), or a physiologically-acceptable salt thereof, mixed with a pharmaceutically-acceptable carrier. In certain embodiments, the pharmaceutical composition of the invention additionally comprises one or more anti-cancer agents (e.g., chemotherapeutic agents). The pharmaceutical formulations of the invention can also comprise at least one miR gene product or miR gene expression-inhibition compound (or at least one nucleic acid comprising a sequence encoding the miR gene product or miR gene expression-inhibition compound), which are encapsulated by liposomes and a pharmaceutically-acceptable carrier.

Pharmaceutical compositions of the invention can also comprise conventional pharmaceutical excipients and/or additives. Suitable pharmaceutical excipients include stabilizers, antioxidants, osmolality adjusting agents, buffers, and pH adjusting agents. Suitable additives include, e.g., physiologically biocompatible buffers (e.g., tromethamine hydrochloride), additions of chelants (such as, for example, DTPA or DTPA-bisamide) or calcium chelate complexes (such as, for example, calcium DTPA, CaNaDTPA-bisamide), or, optionally, additions of calcium or sodium salts (for example, calcium chloride, calcium ascorbate, calcium gluconate or calcium lactate). Pharmaceutical compositions of the invention can be packaged for use in liquid form, or can be lyophilized.

For solid pharmaceutical compositions of the invention, conventional nontoxic solid pharmaceutically-acceptable carriers can be used; for example, pharmaceutical grades of mannitol, lactose, starch, magnesium stearate, sodium saccharin, talcum, cellulose, glucose, sucrose, magnesium carbonate, and the like.

For example, a solid pharmaceutical composition for oral administration can comprise any of the carriers and excipients listed above and 10-95%, preferably 25%-75%, of the at least one miR gene product or miR gene expression-inhibition compound (or at least one nucleic acid comprising sequences encoding them). A pharmaceutical composition for aerosol (inhalational) administration can comprise 0.01-20% by weight, preferably 1%-10% by weight, of the at least one miR gene product or miR gene expression-inhibition compound (or at least one nucleic acid comprising a sequence encoding the miR gene product or miR gene expression-inhibition compound) encapsulated in a liposome as described above, and a propellant. A carrier can also be included as desired; e.g., lecithin for intranasal delivery.

The pharmaceutical compositions of the invention can further comprise one or more anti-cancer agents. In a particular embodiment, the compositions comprise at least one miR gene product or miR gene expression-inhibition compound (or at least one nucleic acid comprising a sequence encoding the miR gene product or miR gene expression-inhibition compound) and at least one chemotherapeutic agent. Chemotherapeutic agents that are suitable for the methods of the invention include, but are not limited to, DNA-alkylating agents, anti-tumor antibiotic agents, anti-metabolic agents, tubulin stabilizing agents, tubulin destabilizing agents, hormone antagonist agents, topoisomerase inhibitors, protein kinase inhibitors, HMG-CoA inhibitors, CDK inhibitors, cyclin inhibitors, caspase inhibitors, metalloproteinase inhibitors, antisense nucleic acids, triple-helix DNAs, nucleic acids aptamers, and molecularly-modified viral, bacterial and exotoxic agents. Examples of suitable agents for the compositions of the present invention include, but are not limited to, cytidine arabinoside, methotrexate, vincristine, etoposide (VP-16), doxorubicin (adriamycin), cisplatin (CDDP), dexamethasone, arglabin, cyclophosphamide, sarcolysin, methylnitrosourea, fluorouracil, 5-fluorouracil (5FU), vinblastine, camptothecin, actinomycin-D, mitomycin C, hydrogen peroxide, oxaliplatin, irinotecan, topotecan, leucovorin, carmustine, streptozocin, CPT-11, taxol, tamoxifen, dacarbazine, rituximab, daunorubicin, 1-β-D-arabinofuranosylcytosine, imatinib, fludarabine, docetaxel and FOLFOX4.

In one embodiment, the method comprises providing a test agent to a cell and measuring the level of at least one miR gene product associated with decreased expression levels in cancerous cells. An increase in the level of the miR gene product in the cell, relative to a suitable control (e.g., the level of the miR gene product in a control cell), is indicative of the test agent being an anti-cancer agent.

Suitable agents include, but are not limited to drugs (e.g., small molecules, peptides), and biological macromolecules (e.g., proteins, nucleic acids). The agent can be produced recombinantly, synthetically, or it may be isolated (i.e., purified) from a natural source. Various methods for providing such agents to a cell (e.g., transfection) are well known in the art, and several of such methods are described hereinabove. Methods for detecting the expression of at least one miR gene product (e.g., Northern blotting, in situ hybridization, RT-PCR, expression profiling) are also well known in the art. Several of these methods are also described herein.

While the invention has been described with reference to various and preferred embodiments, it should be understood by those skilled in the art that various changes may be made and equivalents may be substituted for elements thereof without departing from the essential scope of the invention. In addition, many modifications may be made to adapt a particular situation or material to the teachings of the invention without departing from the essential scope thereof. Therefore, it is intended that the invention not be limited to the particular embodiment disclosed herein contemplated for carrying out this invention, but that the invention will include all embodiments falling within the scope of the claims.

The publication and other material used herein to illuminate the invention or provide additional details respecting the practice of the invention, are incorporated be reference herein, and for convenience are provided in the following bibliography.

### REFERENCES

1. Kopper, L. and J. Timar. 2005. Genomics of lung cancer may change diagnosis, prognosis and therapy. Pathol. Oncol. Res. 11:5-10.

2. Lynch, T. J., D. W. Bell, R. Sordella, S. Gurubhagavatula, R. A. Okimoto, B. W. Brannigan, P. L. Harris, S. M. Haserlat, J. G. Supko, F. G. Haluska, D. N. Louis, D. C. Christiani, J. Settleman, and D. A. Haber. 2004. Activating mutations in the epidermal growth factor receptor underlying responsiveness of non-small-cell lung cancer to gefitinib. N. Engl. J. Med. 350:2129-2139.

3. Shepherd, F. A., P. J. Rodrigues, T. Ciuleanu, E. H. Tan, V. Hirsh, S. Thongprasert, D. Campos, S. Maoleekoonpiroj, M. Smylie, R. Martins, K. M. van, M. Dediu, B. Findlay, D. Tu, D. Johnston, A. Bezjak, G. Clark, P. Santabarbara, and L. Seymour. 2005. Erlotinib in previously treated non-small-cell lung cancer. N. Engl. J. Med. 353:123-132.

4. Sher, Y. P., J. Y. Shih, P. C. Yang, S. R. Roffler, Y. W. Chu, C. W. Wu, C. L. Yu, and K. Peck. 2005. Prognosis of non-small cell lung cancer subjects by detecting circulating cancer cells in the peripheral blood with multiple marker genes. Clin. Cancer Res. 11:173-179.

5. Nana-Sinkam, S. P. and M. W. Geraci. 2006. MicroRNA in lung cancer. Journal of Thoracic Oncology 1:929-931.

6. Johnson, S. M., H. Grosshans, J. Shingara, M. Byrom, R. Jarvis, A. Cheng, E. Labourier, K. L. Reinert, D. Brown, and F. J. Slack. 2005. RAS is regulated by the let-7 microRNA family. Cell 120:635-647.

7. Takamizawa, J., H. Konishi, K. Yanagisawa, S. Tomida, H. Osada, H. Endoh, T. Harano, Y. Yatabe, M. Nagino, Y. Nimura, T. Mitsudomi, and T. Takahashi. 2004. Reduced expression of the let-7 microRNAs in human lung cancers in association with shortened postoperative survival. Cancer Res. 64:3753-3756.

8. Yanaihara, N., N. Caplen, E. Bowman, M. Seike, K. Kumamoto, M. Yi, R. M. Stephens, A. Okamoto, J. Yokota, T. Tanaka, G. A. Calin, C. G. Liu, C. M. Croce, and C. C. Harris. 2006. Unique microRNA molecular profiles in lung cancer diagnosis and prognosis. Cancer Cell 9:189-198.

1. A method of determining whether a subject has, or is at risk for developing, one or more lung cancer associated diseases, comprising:
measuring the level of at least one miR gene product in a peripheral blood sample from the subject,
wherein an alteration in the level of the miR gene product in the sample, relative to the level of a corresponding miR gene product in a control sample, is indicative of the subject either having, or being at risk for developing, one or more lung cancer associated diseases.

2. The method of claim 1, wherein the peripheral blood sample comprises one or more of: whole blood, peripheral blood mononuclear cells (PBMC) and serum.

3. The method of claim 1, wherein the one or more lung cancer associated diseases comprise bronchoalveolar carcinoma (BAC), non-small cell lung cancer (NSCLC), lung adenocarcinoma, and a lung squamous cell carcinoma.

4. The method of claim 1, wherein the peripheral blood sample comprises whole blood, and wherein at least one miR gene product is one or more miR gene products selected from the group shown in Table 1 consisting of an increased miR expression of:
miR
hsa-miR-518f
hsa-miR-516-3,5p
hsa-miR-517b*
hsa-miR-490No2
hsa-miR-139-prec
hsa-miR-007-2-precNo1
hsa-miR-021-prec-17No2
hsa-miR-106bNo2
hsa-miR-345No2
hsa-miR-217-precNo1
hsa-miR-323No2
hsa-miR-218-2-precNo2
hsa-miR-202
hsa-miR-425No1
hsa-miR-096-prec-7No1
hsa-miR-125a-precNo2
hsa-miR-339No1
hsa-miR-141-precNo1
hsa-miR-321No1.

5. The method of claim 4, wherein the miRs are one or more of: hsa-miR-518f and hsa-miR-516-35p.

6. The method of claim 1, wherein the peripheral blood sample comprises whole blood, and wherein at least one miR gene product is one or more miR gene products selected from the group shown in Table 1 consisting of a decreased miR expression of:
miR
hsa-miR-1-2No1
hsa-miR-511-2No2
hsa-miR-101-2No1
hsa-miR-218-2-precNo1
hsa-miR-451No2
hsa-miR-126*No2
hsa-let-7d-v1-prec
hsa-miR-1-1No1
hsa-miR-123-precNo1
hsa-miR-100No1
hsa-miR-150-prec
hsa-miR-021-prec-17No1
hsa-miR-34aNo1
hsa-let-7iNo1
hsa-miR-017-precNo2
hsa-miR-001b-2-prec
hsa-miR-126*No1
hsa-miR-20bNo1
hsa-miR-202-prec
hsa-miR-020-prec
hsa-miR-383No1
hsa-let-7d-v2-precNo2
hsa-let-7g-precNo1
hsa-miR-106aNo1
hsa-miR-126No2
hsa-miR-018-prec
hsa-miR-206-precNo1
hsa-miR-009-1No1
hsa-miR-181c-precNo2
hsa-let-7b-prec
hsa-miR-007-3-precNo1
hsa-miR-103-2-prec
hsa-miR-219-2No2
hsa-miR-016a-chr13
hsa-miR-126No1
hsa-miR-106-prec-X
hsa-miR-107No1
hsa-miR-196-1-precNo1
hsa-miR-106bNo1
hsa-let-7f-1-precNo2
hsa-miR-107-prec-10
hsa-let-7a-1-prec
hsa-miR-144-precNo2
hsa-let-7d-prec
hsa-miR-320No2
hsa-miR-21No1
hsa-miR-103-prec-5=103-1
hsa-miR-516-2No1
hsa-miR-001b-1-prec1
hsa-miR-125b-2-precNo2
hsa-miR-130a-precNo2
hsa-miR-030b-precNo2
hsa-let-7a-2-precNo2
hsa-miR-132-precNo2
hsa-miR-516-45p
hsa-miR-374No1
hsa-miR-015a-2-precNo1
hsa-miR-517a
hsa-miR-016b-chr3
hsa-miR-017-precNo1
hsa-miR-148-prec

7. The method of claim 6, wherein the miRs are one or more of:
miR
hsa-miR-1-2No1
hsa-miR-511-2No2
hsa-miR-101-2No1
hsa-miR-218-2-precNo1
hsa-miR-451No2
hsa-miR-126*No2
hsa-let-7d-v1-prec
hsa-miR-1-1No1
hsa-miR-123-precNo1
hsa-miR-100No1
hsa-miR-150-prec
hsa-miR-021-prec-17No1
hsa-miR-34aNo1
hsa-let-7iNo1
hsa-miR-126*No1
hsa-miR-126No2
hsa-miR-181c-precNo2
hsa-miR-126No1

8. The method of claim 1, wherein the peripheral blood sample comprises peripheral blood mononuclear cells PBMC), and
wherein at least one miR gene product is one or more miR gene products selected from the group shown in Table 2 consisting of a decreased miR expression of: hsa-miR-630.

9. The method of claim 1, wherein the sample comprises peripheral blood mononuclear cells, and
and wherein at least one miR gene product is one or more miR gene products selected from the group shown in Table 2 consisting of an increased miR expression of: hsa-miR-152, hsa-miR-365, hsa-miR-487a, hsa-miR-148a, hsa-miR-636, hsa-miR-320 and hsa-miR-145.

10. The method of claim 1, wherein the peripheral blood sample comprises serum, and
wherein at least one miR gene product is one or more miR gene products selected from the group shown in Table 3 consisting of an increased miR expression of: hsa-miR-192.

11. The method of claim 1, wherein the sample comprises serum, and
wherein at least one miR gene product is one or more miR gene products selected from the group shown in Table 3 consisting of a decreased miR expression of: hsa-miR-532, hsa-miR-197, hsa-miR-342.

12. The method of claim 1, wherein the at least one miR gene product is one or more miR gene products selected from the group shown in Table 4.

13. The method of claim 1, wherein the at least one miR gene product is one or more miR gene products selected from the group shown in Table 5.

14. The method of claim 1, wherein the at least one miR gene product is one or more miR gene products selected from the group shown in Table 6.

15. The method of claim 1, wherein the method is used for determining the prognosis of a subject with lung cancer, comprising:
measuring the level of at least one miR gene product in the sample from the subject,
wherein the miR gene product is associated with an adverse prognosis in lung cancer; and,
an alteration in the level of the at least one miR gene product in the sample, relative to the level of a corresponding miR gene product in a control sample, is indicative of an adverse prognosis.

16. A method of detecting one or more lung cancer associated diseases in a peripheral blood sample, the method comprising:
analyzing the sample for the altered expression of at least one biomarker associated with lung cancer, and
correlating the altered expression of the at least one biomarker with the presence or absence of lung cancer in the sample,
wherein the at least one biomarker is selected from the miRs listed in Table 1, Table 2 or Table 3.

17. A method of early diagnosing a subject suspected of having one or more lung cancer associated diseases, the method comprising:
obtaining a peripheral blood sample from the subject;
analyzing the sample for the altered expression of at least one biomarker associated with lung cancer;
correlating the altered expression of at least one biomarker with the presence of lung cancer in the subject;
wherein the at least one biomarker is selected from the miRs listed in Table 1, Table 2 or Table 3.

18. (canceled)

19. (canceled)

20. A method of comparing peripheral blood samples in a patient having undergone chemoradiation therapy for one or more lung cancer associated diseases and samples of patients not having undergone chemoradiation therapy, comprising:
comparing differential expression of at least one of biomarker selected from the group consisting of the miRs listed in Table 1, Table 2 or Table 3.

21. A method of comparing staging in one or more lung cancer associated diseases in a patient, comprising:
obtaining a peripheral blood sample from the patient; and comparing differential expression of at least one of biomarker selected from the group consisting of the miRs listed in Table 1, Table 2 or Table 3.

22. (canceled)

23. (canceled)

24. (canceled)

25. (canceled)

26. (canceled)

27. (canceled)

28. (canceled)

29. (canceled)

30. (canceled)

31. (canceled)

32. (canceled)

33. (canceled)

34. A kit for screening for a candidate compound for a therapeutic agent to treat one or more lung cancer associated diseases, wherein the kit comprises: one or more reagents of at least one miR listed in Table 1, Table 2 or Table 3 and a cell expressing at least one miR.

35. The kit of claim 34, wherein the presence of the miR is detected using a reagent comprising an antibody or an antibody fragment which specifically binds with at least one miR.

36. (canceled)

37. (canceled)

38. (canceled)

39. (canceled)

40. (canceled)
